(19) United States
(12) Patent Application Publication
(10) Pub. No.: US 2020/0190081 A1
(43) Pub. Date: Jun. 18, 2020

(54) META SUBSTITUTED PHENYLPYRAZOLO- AND PHENYLPYRROLO- PYRIDAZINE DERIVATIVES HAVING MULTIMODAL ACTIVITY AGAINST PAIN

(71) Applicant: ESTEVE PHARMACEUTICALS, S.A., Barcelona (ES)

(72) Inventors: Marina VIRGILI-BERNADO, Barcelona (ES); Monica ALONSO-XALMA, Barcelona (ES); Carmen ALMANSA-ROSALES, Barcelona (ES); José-Luis DÍAZ-FERNÁNDEZ, Manresa (ES)

(21) Appl. No.: 16/464,345

(22) PCT Filed: Nov. 30, 2017
(86) PCT No.: PCT/EP2017/080938

§ 371 Date: May 28, 2019

(30) Foreign Application Priority Data
Nov. 30, 2016 (EP) 16382578.9

(51) Int. Cl. C07D 471/04; C07D 473/02

(52) U.S. Cl. CPC C07D 471/04; C07D 473/02

(57) ABSTRACT

The present invention relates to meta substituted phenylpyrazolo- and phenylpyrrolo-pyridazine derivatives having dual pharmacological activity towards both the α2δ subunit, in particular the α2δ-1 subunit, of the voltage-gated calcium channel and the NET receptor, to processes of preparation of such compounds, to pharmaceutical compositions comprising them, and to their use in therapy, in particular for the treatment of pain.

### FIELD OF THE INVENTION

The present invention relates to compounds having dual pharmacological activity towards both the α₂δ subunit of the voltage-gated calcium channel, and noradrenaline transporter (NET) and more particularly to meta substituted phenylpyrazolo- and phenylpyrrolo-pyridazine derivatives having this pharmacological activity, to processes of preparation of such compounds, to pharmaceutical compositions comprising them, and to their use in therapy, in particular for the treatment of pain.

### BACKGROUND OF THE INVENTION

The adequate management of pain constitutes an important challenge, since currently available treatments provide in many cases only modest improvements, leaving many patients unrelieved (Turk, D. C., Wilson, H. D., Cahana, A.; 2011; Lancet; 377; 2226-2235). Pain affects a big portion of the population with an estimated prevalence of 20% and its incidence, particularly in the case of chronic pain, is increasing due to the population ageing. Additionally, pain is clearly related to comorbidities, such as depression, anxiety and insomnia, which leads to important productivity losses and socio-economical burden (Goldberg, D. S., McGee, S. J.; 2011; BMC Public Health; 11; 770). Existing pain therapies include non-steroidal anti-inflammatory drugs (NSAIDs), opioid agonists, calcium channel blockers and antidepressants, but they are much less than optimal regarding their safety ratio. All of them show limited efficacy and a range of secondary effects that preclude their use, especially in chronic settings.

Voltage-gated calcium channels (VGCC) are required for many key functions in the body. Different subtypes of voltage-gated calcium channels have been described (Zamponi et al., Pharmacol. Rev. 2015 67:821-70). The VGCC are assembled through interactions of different subunits, namely α₁ (Caᵥα₁), β (Caᵥβ) α₂δ (Caᵥα₂δ) and γ (Caᵥγ). The α₁ subunits are the key porous forming units of the channel complex, being responsible for the Ca²⁺ conduction and generation of Ca²⁺ influx. The α₂δ, α, and γ subunits are auxiliary, although very important for the regulation of the channel, since they increase the expression of the α₁ subunits in the plasma membrane as well as modulate their function, resulting in functional diversity in different cell types. Based on their physiological and pharmacological properties, VGCC can be subdivided into low voltage-activated T-type (Caᵥ3.1, Caᵥ3.2, and Caᵥ3.3), and high voltage-activated L-(Caᵥ1.1 through Caᵥ1.4), N—(Caᵥ2.2), P/Q-(Caᵥ2.1), and R—(Caᵥ2.3) types, depending on the channel forming Cava subunits. All of these five subclasses are found in the central and peripheral nervous systems. Regulation of intracellular calcium through activation of these VGCC plays obligatory roles in: 1) neurotransmitter release, 2) membrane depolarization and hyperpolarization, 3) enzyme activation and inactivation, and 4) gene regulation (Perret and Luo, Neurotherapeutics. 2009 6:679-92; Zamponi et al., 2015 supra; Neumaier et al., Prog. Neurobiol. 2015 129:1-36.). A large body of data has clearly indicated that VGCC are implicated in mediating various disease states including pain processing. Drugs interacting with the different calcium channel subtypes and subunits have been developed. Current therapeutic agents include drugs targeting L-type Caᵥ1.2 calcium channels, particularly 1,4-dihydropyridines, which are widely used in the treatment of hypertension. T-type (Caᵥ3) channels are the target of ethosuximide, widely used in absence epilepsy. Ziconotide, a peptide blocker of N-type (Caᵥ2.2) calcium channels, has been approved as a treatment of intractable pain. (Perret and Luo, 2009, supra; Vink and Alewood, Br J Pharmacol. 2012 167:970-89.).

The Caᵥ1 and Caᵥ2 subfamilies contain an auxiliary α₂δ subunit, which is the therapeutic target of the gabapentinoid drugs of value in certain epilepsies and chronic neuropathic pain. To date, there are four known α₂δ subunits, each encoded by a unique gene and all possessing splice variants. Each α₂δ protein is encoded by a single messenger RNA and is posttranslationally cleaved and then linked by disulfide bonds. Four genes encoding α₂δ subunits have now been cloned. α₂δ-1 was initially cloned from skeletal muscle and shows a fairly ubiquitous distribution. The α₂δ-2 and α₂δ-3 subunits were subsequently cloned from brain. The most recently identified subunit, α₂δ-4, is largely nonneuronal. The human α₂δ-4 protein sequence shares 30, 32 and 61% identity with the human α₂δ-1, α₂δ-2 and α₂δ-3 subunits, respectively. The gene structure of all α₂δ subunits is similar. All α₂δ subunits show several splice variants (Davies et al., Trends Pharmacol Sci. 2007 28:220-8.; Dolphin A C, Nat Rev Neurosci. 2012 13:542-55., Biochim Biophys Acta. 2013 1828:1541-9.).

The Caᵥα₂δ-1 subunit may play an important role in neuropathic pain development (Perret and Luo, 2009, supra; Vink and Alewood, 2012, supra). Biochemical data have indicated a significant Caᵥα₂δ-1, but not Caᵥα₂δ-2, subunit upregulation in the spinal dorsal horn, and DRG (dorsal root ganglia) after nerve injury that correlates with neuropathic pain development. In addition, blocking axonal transport of injury-induced DRG Caᵥα₂δ-1 subunit to the central presynaptic terminals diminishes tactile allodynia in nerve injured animals, suggesting that elevated DRG Caᵥα₂δ-1 subunit contributes to neuropathic allodynia.

The Caᵥα₂δ-1 subunit (and the Caᵥα₂δ-2, but not Caᵥα₂δ-3 and Caᵥα₂δ-4, subunits) is the binding site for gabapentin which has anti-allodynic/hyperalgesic properties in patients and animal models. Because injury-induced Caᵥα₂δ-1 expression correlates with neuropathic pain development and maintenance, and various calcium channels are known to contribute to spinal synaptic neurotransmission and DRG neuron excitability, injury-induced Caᵥα₂δ-1 subunit upregulation may contribute to the initiation and maintenance of neuropathic pain by altering the properties and/or distribution of VGCC in the subpopulation of DRG neurons and their central terminals, therefore modulating excitability and/or synaptic neuroplasticity in the dorsal horn. Intrathecal antisense oligonucleotides against the Caᵥα₂δ-1 subunit can block nerve injury-induced Caᵥα₂δ-1 upregulation and prevent the onset of allodynia and reserve established allodynia.

As mentioned above, the α₂δ subunits of VGCC form the binding site for gabapentin and pregabalin, which are structural derivatives of the inhibitory neurotransmitter GABA although they do not bind to GABAA, GABAB, or benzodiazepine receptors, or alter GABA regulation in animal brain preparations. The binding of gabapentin and pregabalin to the Caᵥα₂δ subunit results in a reduction in the calcium-dependent release of multiple neurotransmitters, leading to efficacy and tolerability for neuropathic pain management. Gabapentinoids may also reduce excitability by inhibiting synaptogenesis (Perret and Luo, 2009, supra; Vink and Alewood, 2012, supra, Zamponi et al., 2015, supra).

It is also known that Noradrenaline (NA), also called norepinephrine, functions in the human brain and body as a hormone and neurotransmitter. Noradrenaline exerts many effects and mediates a number of functions in living organisms. The effects of noradrenaline are mediated by two distinct super-families of receptors, named alpha- and beta-adrenoceptors. They are further divided into subgroups exhibiting specific roles in modulating behavior and cognition of animals. The release of the neurotransmitter noradrenaline throughout the mammalian brain is important for modulating attention, arousal, and cognition during many behaviors (Mason, S. T.; Prog. Neurobiol.; 1981; 16; 263-303).

The noradrenaline transporter (NET, SLC6A2) is a monoamine transporter mostly expressed in the peripheral and central nervous systems. NET recycles primarily NA, but also serotonin and dopamine, from synaptic spaces into presynaptic neurons. NET is a target of drugs treating a variety of mood and behavioral disorders, such as depression, anxiety, and attention-deficit/hyperactivity disorder (ADHD). Many of these drugs inhibit the uptake of NA into the presynaptic cells through NET. These drugs therefore increase the availability of NA for binding to postsynaptic receptors that regulate adrenergic neurotransmission. NET inhibitors can be specific. For example, the ADHD drug atomoxetine is a NA reuptake inhibitor (NRI) that is highly selective for NET. Reboxetine was the first NRI of a new antidepressant class (Kasper et al.; Expert Opin. Pharmacother.; 2000; 1; 771-782). Some NET inhibitors also bind multiple targets, increasing their efficacy as well as their potential patient population.

For instance, the antidepressants venlafaxine and duloxetine are dual reuptake inhibitor of serotonin and NA that targets both NET and the serotonin transporter (SERT, SLC6A4). Duloxetine has been licensed for major depressive disorder, generalised anxiety disorder, diabetic peripheral neuropathic pain, fibromyalgia and chronic musculoskeletal pain.

Endogenous, descending noradrenergic fibers impose analgesic control over spinal afferent circuitry mediating the transmission of pain signals (Ossipov et al.; J. Clin. Invest.; 2010; 120; 3779-3787). Alterations in multiple aspects of noradrenergic pain processing have been reported, especially in neuropathic pain states (Ossipov et al., 2010; Wang et al.; J. Pain; 2013; 14; 845-853). Numerous studies have demonstrated that activation of spinal α₂-adrenergic receptors exerts a strong antinociceptive effect. Spinal clonidine blocked thermal and capsaicin-induced pain in healthy human volunteers (Ossipov et a, 2010). Noradrenergic reuptake inhibitors have been used for the treatment of chronic pain for decades: most notably the tricyclic antidepressants, amitriptyline, and nortriptyline. Once released from the presynaptic neuron, NA typically has a short-lived effect, as much of it is rapidly transported back into the nerve terminal. In blocking the reuptake of NA back into the presynaptic neurons, more neurotransmitter remains for a longer period of time and is therefore available for interaction with pre- and postsynaptic α₂-adrenergic receptors (AR). Tricyclic antidepressants and other NA reuptake inhibitors enhance the antinociceptive effect of opioids by increasing the availability of spinal NA. The α₂A-AR subtype is necessary for spinal adrenergic analgesia and synergy with opioids for most agonist combinations in both animal and humans (Chabot-Doré et al.; Neuropharmacology; 2015; 99; 285-300). A selective upregulation of spinal NET in a rat model of neuropathic pain with concurrent downregulation of serotonin transporters has been shown (Fairbanks et al.; Pharmacol. Ther.; 2009; 123; 224-238). Inhibitors of NA reuptake such as nisoxetine, nortriptyline and maprotiline and dual inhibitors of the noradrenaline and serotonin reuptake such as imipramine and milnacipran produce potent anti-nociceptive effects in the formalin model of tonic pain. Neuropathic pain resulting from the chronic constriction injury of the sciatic nerve was prevented by the dual uptake inhibitor, venlafaxine. In the spinal nerve ligation model, amitriptyline, a non-selective serotonin and noradrenaline reuptake blocker, the preferential noradrenaline reuptake inhibitor, desipramine and the selective serotonin and noradrenaline reuptake inhibitors, milnacipran and duloxetine, produce a decrease in pain sensitivity whereas the selective serotonin reuptake inhibitor, fluoxetine, is ineffective (Mochizucki, D.; Psychopharmacol.; 2004; Supplm. 1; S15-S19; Hartrick, C. T.; Expert Opin. Investig. Drugs; 2012; 21; 1827-1834). A number of nonselective investigational agents focused on noradrenergic mechanisms with the potential for additive or even synergistic interaction between multiple mechanisms of action are being developed.

Polypharmacology is a phenomenon in which a drug binds multiple rather than a single target with significant affinity. The effect of polypharmacology on therapy can be positive (effective therapy) and/or negative (side effects). Positive and/or negative effects can be caused by binding to the same or different subsets of targets; binding to some targets may have no effect. Multi-component drugs or multi-targeting drugs can overcome toxicity and other side effects associated with high doses of single drugs by countering biological compensation, allowing reduced dosage of each compound or accessing context-specific multitarget mechanisms. Because multitarget mechanisms require their targets to be available for coordinated action, one would expect synergies to occur in a narrower range of cellular phenotypes given differential expression of the drug targets than would the activities of single agents. In fact, it has been experimentally demonstrated that synergistic drug combinations are generally more specific to particular cellular contexts than are single agent activities, such selectivity is achieved through differential expression of the drugs' targets in cell types associated with therapeutic, but not toxic, effects (Lehar et al., Nat. Biotechnol. 2009; 27: 659-666.).

In the case of chronic pain, which is a multifactorial disease, multi-targeting drugs may produce concerted pharmacological intervention of multiple targets and signaling pathways that drive pain. Because they actually make use of biological complexity, multi-targeting (or multi-component drugs) approaches are among the most promising avenues toward treating multifactorial diseases such as pain (Gilron et al., Lancet Neurol. 2013 November; 12(11):1084-95.). In fact, positive synergistic interaction for several compounds, including analgesics, has been described (Schröder et al., J Pharmacol. Exp. Ther. 2011; 337:312-20. Erratum in: J Pharmacol. Exp. Ther. 2012; 342: 232; Zhang et al., Cell Death Dis. 2014; 5: e1138; Gilron et al., 2013, supra).

Given the significant differences in pharmacokinetics, metabolisms and bioavailability, reformulation of drug combinations (multi-component drugs) is challenging. Further, two drugs that are generally safe when dosed individually cannot be assumed to be safe in combination. In addition to the possibility of adverse drug-drug interactions, if the theory of network pharmacology indicates that an effect on phenotype may derive from hitting multiple targets, then that combined phenotypic perturbation may be efficacious or deleterious. The major challenge to both drug combination strategies is the regulatory requirement for each individual drug to be shown to be safe as an individual agent and in combination (Hopkins, Nat. Chem. Biol. 2008; 4:682-90).

An alternative strategy for multitarget therapy is to design a single compound with selective polypharmacology (multi-targeting drug). It has been shown that many approved drugs act on multiple targets. Dosing with a single compound may have advantages over a drug combination in terms of equitable pharmacokinetics and biodistribution. Indeed, troughs in drug exposure due to incompatible pharmacokinetics between components of a combination therapy may create a low-dose window of opportunity where a reduced selection pressure can lead to drug resistance. In terms of drug registration, approval of a single compound acting on multiple targets faces significantly lower regulatory barriers than approval of a combination of new drugs (Hopkins, 2008, supra).

Thus, the present application, relates to the advantages of dual inhibition of noradrenaline transporter (NET) and the α₂δ-1 subunit of voltage-gated calcium channels, in the same molecule to treat chronic pain.

There are two potentially important interactions between NET and α₂δ-1 inhibition: 1) synergism in analgesia, thus reducing the risk of specific side effects; and 2) inhibition of pain-related affective comorbidities such as anxiety and/or depressive like behaviors (Nicolson et al.; Harv. Rev. Psychiatry; 2009; 17; 407-420).
1) Preclinical research has demonstrated that gabapentinoids attenuated pain-related behaviors through supraspinal activation of the descending noradrenergic system (Tanabe et al.; J. Neurosci. Res.; 2008; Hayashida, K.; Eur. J. Pharmacol.; 2008; 598; 21-26). In consequence, the α₂δ-1-related analgesia mediated by NA-induced activation of spinal α2-adrenergic receptors can be potentiated by the inhibition of the NET. Some evidence from combination studies in preclinical models of neuropathic pain exist. Oral duloxetine with gabapentin was additive to reduce hypersensitivity induced by nerve injury in rats (Hayashida & Eisenach, 2008). The combination of gabapentin and nortriptyline was synergic in mice submitted to orofacial pain and to peripheral nerve injury model (Miranda, H. F. et al.; J. Orofac. Pain; 2013; 27; 361-366; Pharmacology; 2015; 95; 59-64).
2) Drug modulation of NET and α₂δ-1 has been shown to produce antidepressant and anti-anxiety effects respectively (Frampton, J. E.; CNS Drugs; 2014; 28; 835-854; Hajós, M. et al.; CNS Drug Rev.; 2004; 10; 23-44). In consequence, a dual drug that inhibited the NET and α₂δ-1 subunit of VGCC may also stabilize pain-related mood impairments by acting directly on both physical pain and the possible mood alterations.

Pain is multimodal in nature, since in nearly all pain states several mediators, signaling pathways and molecular mechanisms are implicated. Consequently, monomodal therapies fail to provide complete pain relief. Currently, combining existing therapies is a common clinical practice and many efforts are directed to assess the best combination of available drugs in clinical studies (Mao, J., Gold, M. S., Backonja, M.; 2011; J. Pain; 12; 157-166).

Accordingly, there is a need to find compounds that have an alternative or improved pharmacological activity in the treatment of pain, being both effective and showing the desired selectivity, and having good “drugability” properties, i.e. good pharmaceutical properties related to administration, distribution, metabolism and excretion.

The authors of the present invention, have found a serie of compounds that show dual pharmacological activity towards both the α₂δ subunit, in particular the α₂δ-1 subunit, of the voltage-gated calcium channel, and the noradrenaline transporter (NET), resulting in an innovative, effective and alternative solution for the treatment of pain.

In view of the existing results of the currently available therapies and clinical practices, the present invention offers a solution by combining in a single compound binding to two different targets relevant for the treatment of pain. This was mainly achieved by providing the compounds according to the invention that bind both to the noradrenaline transporter (NET) and to the α₂δ subunit, in particular the α₂δ-1 subunit, of the voltage-gated calcium channel.

### SUMMARY OF THE INVENTION

In this invention a family of structurally distinct meta substituted phenylpyrazolo- and phenylpyrrolo-pyridazine derivatives, encompassed by formula (I), which have a dual pharmacological activity towards both the α₂δ subunit, in particular the α₂δ-1 subunit, of the voltage-gated calcium channel, and the noradrenaline transporter (NET) was identified, thus solving the above problem of identifying alternative or improved pain treatments by offering such dual compounds.

The present invention discloses novel compounds with affinity to α2δ subunit of voltage-gated calcium channels, more specifically to the α2δ-1, and also have inhibitory effect towards noradrenaline transporter (NET), thus resulting in a dual activity for treating pain and pain related disorders.

It has to be noted, though, that functionalities “antagonism” and “agonism” are also sub-divided in their effect into subfunctionalities like partial agonism or inverse agonism. Accordingly, the functionalities of the compounds should be considered within a relatively broad bandwidth.

An antagonist blocks or dampens agonist-mediated responses. Known subfunctionalities are neutral antagonists or inverse agonists.

An agonist increases the activity of the receptor above its basal level. Known subfunctionalities are full agonists, or partial agonists.

The main object of the invention is directed to a compound having a dual activity binding to the α₂δ subunit, in particular the α₂δ-1 subunit, of the voltage-gated calcium channel and the noradrenaline transporter (NET) and the α₂δ-1 subunit of voltage-gated calcium channels, for use in the treatment of pain.

As this invention is aimed at providing a compound or a chemically related series of compounds which act as dual ligands of the α₂δ subunit, in particular the α₂δ-1 subunit, of the voltage-gated calcium channel and the noradrenaline transporter (NET), it is a very preferred embodiment if the compound has a binding expressed as Kᵢ responding to the following scales:

Kᵢ(NET) is preferably <1000 nM, more preferably <500 nM, even more preferably <100 nM.
Kᵢ(α₂δ-1) is preferably <10000 nM, more preferably <5000 nM, even more preferably <500 nM or even more preferably <100 nM.

The invention is directed in a main aspect to a compound of general Formula (I),

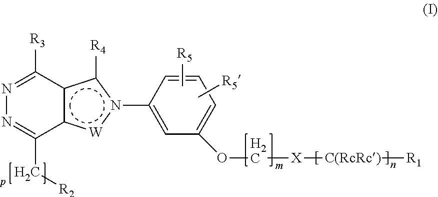

wherein R₁, R₂, R₃, R₄, R₅, R_{5′}, R_{c}, R_{c′}, X, W, m, n and p are as defined below in the detailed description.

A further object of the invention refers to the processes for preparation of compounds of general formula (I).

A still further object of the invention refers to the use of intermediate compounds for the preparation of a compound of general formula (I).

It is also an object of the invention a pharmaceutical composition comprising a compound of formula (I).

Finally, it is an object of the invention the use of compound as a medicament and more particularly for the treatment of pain and pain related conditions.

### DETAILED DESCRIPTION OF THE INVENTION

The invention is directed to a family of structurally distinct meta substituted phenylpyrazolo- and phenylpyrrolo-pyridazine derivatives which have a dual pharmacological activity towards both the α₂δ subunit, in particular the α₂δ-1 subunit, of the voltage-gated calcium channel and the NET receptor.

The invention is directed to compounds having a dual activity binding to the α₂δ subunit, in particular the α₂δ-1 subunit, of the voltage-gated calcium channel and the NET receptor for use in the treatment of pain.

As this invention is aimed at providing a compound or a chemically related series of compounds which act as dual ligands of the α₂δ subunit, in particular the α₂δ-1 subunit, of the voltage-gated calcium channel and the NET receptor it is a preferred embodiment if the compound has a binding expressed as Kᵢ responding to the following scales:

Kᵢ(NET) is preferably <1000 nM, more preferably <500 nM, even more preferably <100 nM.
Kᵢ(α₂δ-1) is preferably <10000 nM, more preferably <5000 nM, even more preferably <500 nM or even more preferably <100 nM.

In its broader aspect, the present invention is directed to compounds of general Formula (I):

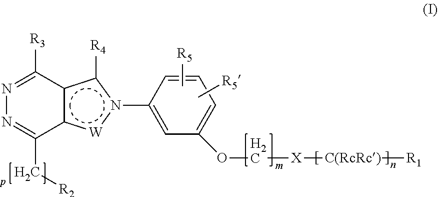

wherein
m is 0, 1, 2, 3 or 4;
n is 0, 1, 2, 3 or 4;
p is 0, 1, 2, 3 or 4;
W is nitrogen or —C(R_{4′})—;
X is selected from a bond, substituted or unsubstituted aryl or —C(RₓR_{x′})—;
Rₓ is selected from substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
Rₓ, is selected from hydrogen, substituted or unsubstituted C₁₋₆ alkyl, substituted or unsubstituted C₂₋₆ alkenyl and substituted or unsubstituted C₂₋₆ alkynyl;
   R₁ is selected from —NR₆R_{6′} and substituted or unsubstituted N-containing-heterocyclyl;
wherein R₆ and R_{6′} are independently selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
   R₂ is selected from hydrogen, —NR₇R_{7′}, —CN, —CHR₇R_{7′} and substituted or unsubstituted heterocyclyl;
wherein R₇ and R_{7′} are independently selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
   R₃ is selected from hydrogen, substituted or unsubstituted C₁₋₆ alkyl, substituted or unsubstituted C₂₋₆ alkenyl and substituted or unsubstituted C₂₋₆ alkynyl;
   R₄ is selected from hydrogen, substituted or unsubstituted C₁₋₆ alkyl, substituted or unsubstituted C₂₋₆ alkenyl and substituted or unsubstituted C₂₋₆ alkynyl;
   R_{4′} is selected from hydrogen, substituted or unsubstituted C₁₋₆ alkyl, substituted or unsubstituted C₂₋₆ alkenyl and substituted or unsubstituted C₂₋₆ alkynyl;
   R₅ and R_{5′}, are independently selected from hydrogen, halogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl, substituted or unsubstituted alkyheterocyclyl, —OR₈, —NO₂, —NR₈R_{8′}, —NR₈C(O)R_{8′}, —NR₈S(O)₂R_{8′}, —S(O)₂NR₈R_{8′}, —NR₈C(O)NR_{8′}R_{8″}, —SR₈, —S(O)R₈, S(O)₂R₈, —CN, haloalkyl, haloalkoxy, —C(O)OR₈, —C(O)NR₈R_{8′}, —OCH₂CH₂OR₈, —NR₈S(O)₂NR_{8′}R_{8″}, and C(CH₃)₂OR₈;
wherein R₈, R_{8′}, and R_{8″} are independently selected from hydrogen, unsubstituted C₁₋₆ alkyl, unsubstituted C₂₋₆ alkenyl, and unsubstituted C₂₋₆ alkynyl;
   R_{c} and R_{c′} are independently selected from hydrogen, substituted or unsubstituted C₁₋₆ alkyl, substituted or unsubstituted C₂₋₆ alkenyl and substituted or unsubstituted C₂₋₆ alkynyl;
   alternatively, R_{c} and R_{c′} may form with the carbon atom to which they are attached, a substituted or unsubstituted cycloalkyl;

These compounds according to the invention are optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment, these compounds according to the invention are optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof.

In a particular embodiment the following proviso applies:

—[CH₂]ₘ—X—[C(R_{c}R_{c′})]ₙ—R₁ is attached to the oxygen atom through a carbon atom.

In a further embodiment the compound according to the invention of general Formula (I) is a compound of general Formula (I′)

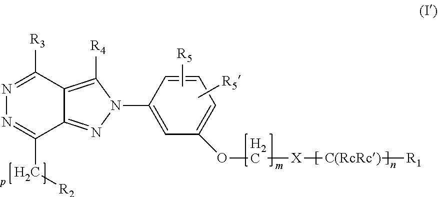

wherein R₁, R₂, R₃, R₄, R₅, R_{5′}, R_{c}, R_{c′}, X, m, n and p are as defined below in the detailed description,
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound of general Formula (I′)

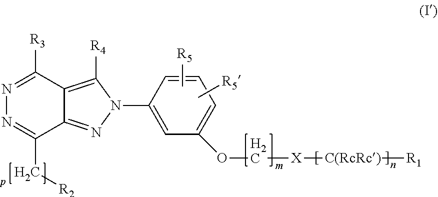

wherein
m is 0, 1, 2, 3 or 4;
n is 0, 1, 2, 3 or 4;
p is 0, 1, 2, 3 or 4;
X is selected from a bond, substituted or unsubstituted aryl or —C(RₓR_{x′})—;
Rₓ is selected from substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
R_{x′} is selected from hydrogen, substituted or unsubstituted C₁₋₆ alkyl, substituted or unsubstituted C₂₋₆ alkenyl and substituted or unsubstituted C₂₋₆ alkynyl;
   R₁ is selected from —NR₆R_{6′}, and substituted or unsubstituted N-containing-heterocyclyl;
wherein R₆ and R_{6′}, are independently selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
   R₂ is selected from hydrogen, —NR₇R_{7′}, —CN, —CHR₇R_{7′} and substituted or unsubstituted heterocyclyl;
wherein R₇ and R_{7′} are independently selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
   R₃ is selected from hydrogen, substituted or unsubstituted C₁₋₆ alkyl, substituted or unsubstituted C₂₋₆ alkenyl and substituted or unsubstituted C₂₋₆ alkynyl;
   R₄ is selected from hydrogen, substituted or unsubstituted C₁₋₆ alkyl, substituted or unsubstituted C₂₋₆ alkenyl and substituted or unsubstituted C₂₋₆ alkynyl;
   R_{4′}, is selected from hydrogen, substituted or unsubstituted C₁₋₆ alkyl, substituted or unsubstituted C₂₋₆ alkenyl and substituted or unsubstituted C₂₋₆ alkynyl;
   R₅ and R_{5′} are independently selected from hydrogen, halogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl, substituted or unsubstituted alkyheterocyclyl, —OR₈, —NO₂, —NR₈R_{8′}, —NR₈C(O)R_{8′}, —NR₈S(O)₂R_{8′}, —S(O)₂NR₈R_{8′}, —NR₈C(O)NR_{8′}R_{8″}, —SR₈, —S(O)R₈, S(O)₂R₈, —CN, haloalkyl, haloalkoxy, —C(O)OR₈, —C(O)NR₈R_{8′}, —OCH₂CH₂OR₈, —NR₈S(O)₂NR_{8′}R_{8″}, and C(CH₃)₂OR₈;
wherein R₈, R_{8′} and R_{8″} are independently selected from hydrogen, unsubstituted C₁₋₆ alkyl, unsubstituted C₂₋₆ alkenyl, and unsubstituted C₂₋₆ alkynyl;
   R_{c} and R_{c′} are independently selected from hydrogen, substituted or unsubstituted C₁₋₆ alkyl, substituted or unsubstituted C₂₋₆ alkenyl and substituted or unsubstituted C₂₋₆ alkynyl;
   alternatively, R_{c} and R_{c40} may form with the carbon atom to which they are attached, a substituted or unsubstituted cycloalkyl;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound of general Formula (I²′)

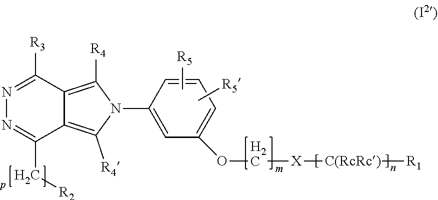

wherein R₁, R₂, R₃, R₄, R₅, R_{5′}, R_{c}, R_{c′}, X, m, n and p are as defined below in the detailed description,
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound of general Formula (I²′)

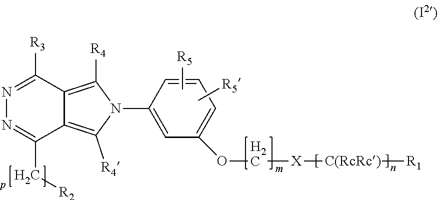

wherein
m is 0, 1, 2, 3 or 4;
n is 0, 1, 2, 3 or 4;
p is 0, 1, 2, 3 or 4;
X is selected from a bond, substituted or unsubstituted aryl or —C(RₓR_{x′})—;
Rₓ is selected from substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
R_{x′} is selected from hydrogen, substituted or unsubstituted C₁₋₆ alkyl, substituted or unsubstituted C₂₋₆ alkenyl and substituted or unsubstituted C₂₋₆ alkynyl;
   R₁ is selected from —NR₆R_{6′} and substituted or unsubstituted N-containing-heterocyclyl;
wherein R₆ and R_{6′} are independently selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
   R₂ is selected from hydrogen, —NR₇R_{7′}, —CN, —CHR₇R_{7′} and substituted or unsubstituted heterocyclyl;
wherein R₇ and R_{7′} are independently selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
   R₃ is selected from hydrogen, substituted or unsubstituted C₁₋₆ alkyl, substituted or unsubstituted C₂₋₆ alkenyl and substituted or unsubstituted C₂₋₆ alkynyl;
   R₄ is selected from hydrogen, substituted or unsubstituted C₁₋₆ alkyl, substituted or unsubstituted C₂₋₆ alkenyl and substituted or unsubstituted C₂₋₆ alkynyl;
   R_{4′} is selected from hydrogen, substituted or unsubstituted C₁₋₆ alkyl, substituted or unsubstituted C₂₋₆ alkenyl and substituted or unsubstituted C₂₋₆ alkynyl;
   R₅ and R_{5′} are independently selected from hydrogen, halogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl, substituted or unsubstituted alkyheterocyclyl, —OR₈, —NO₂, —NR₈R_{8′}, —NR₈C(O)R_{8′}, —NR₈S(O)₂R_{8′}, —S(O)₂NR₈R_{8′}, —NR₈C(O)NR_{8′}R_{8″}, —SR₈, —S(O)R₈, S(O)₂R₈, —CN, haloalkyl, haloalkoxy, —C(O)OR₈, —C(O)NR₈R_{8′}, —OCH₂CH₂OR₈, —NR₈S(O)₂NR_{8′}R_{8″}, and C(CH₃)₂OR₈;
wherein R₈, R_{8′} and R_{8″} are independently selected from hydrogen, unsubstituted C₁₋₆ alkyl, unsubstituted C₂₋₆ alkenyl, and unsubstituted C₂₋₆ alkynyl;
   R_{c} and R_{c′} are independently selected from hydrogen, substituted or unsubstituted C₁₋₆ alkyl, substituted or unsubstituted C₂₋₆ alkenyl and substituted or unsubstituted C₂₋₆ alkynyl;
   alternatively, R_{c} and R_{c′} may form with the carbon atom to which they are attached, a substituted or unsubstituted cycloalkyl;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

For clarity purposes, all groups and definitions described in the present description and referring to compounds of general Formula (I), also apply to compounds of general Formulae (I′) and (I²′), (where applicable), and to all intermediate of synthesis, when those groups are present in the mentioned general Markush formulae, since compounds of general Formulae (I′) and (I²′) are included within the scope of the larger definition of general Formula (I).

For clarity purposes, the general Markush Formula (I)

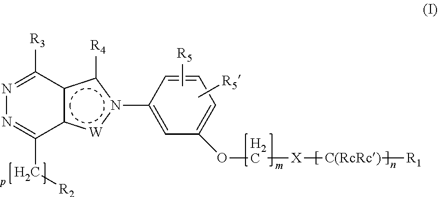

is equivalent to

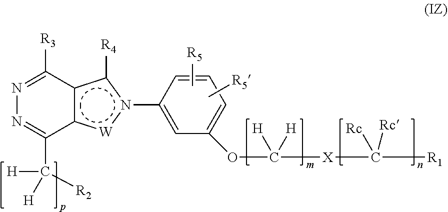

wherein only —C(R_{C}R_{C′})— and —CH₂— are included into the brackets, and n, m and p mean the number of times that —C(R_{C}R_{C′})— and —CH₂— are repeated, respectively. The same would apply, when applicable, to general Markush Formulae (I′) and (I²′), and to all intermediates of synthesis.

In addition, and for clarity purposes, it should further be understood that naturally if m is 0, the oxygen and/or X are still present in general Markush Formulae (I), (I′) and (I²′). In the same way when n is 0, R₁ and/or X are still present, when applicable, in general Markush Formulae (I), (I′) and (I²′) and to all intermediates of synthesis. In the same way when p is 0, R₂ is still present, when applicable, in general Markush Formulae (I), (I′) and (I²′), and to all intermediates of synthesis.

For clarity purposes, the expression “the cycloalkyl in R_{c} and R_{c′}” means the cycloalkyl resulting when R_{c} and R_{c′} form, together with the carbon to which they are attached, a cycloalkyl. This cycloalkyl can then be substituted or not.

For clarity purposes, reference is also made to the following statements below in the definitions of substitutions on alkyl etc. or aryl etc. that “wherein when different radicals R₁ to R_{14″}, are present simultaneously in Formula (I) they may be identical or different”. This statement is reflected in the below general Formula (I^{3′}) being derived from and falling into general Formula (I),

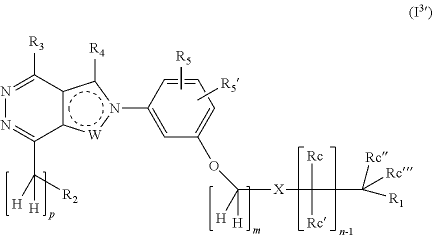

wherein R₁, R₂, R₃, R₄, R₅, R_{5′}, R_{c}, R_{c′}, X, W, m, n and p are as defined in the description. In addition, R_{c″} and R_{c′″} are added. As said above, this statement is thus reflected in that R_{c}, and R_{c′″} are or could be different from R_{c} and R_{c′} or not.

The same would be applicable mutatis mutandis for general Formulas like general Formula (I) as well as the other general Formulas (I¹) to (I²′) above and to all intermediates of synthesis.

In the context of this invention, alkyl is understood as meaning saturated, linear or branched hydrocarbons, which may be unsubstituted or substituted once or several times. It encompasses e.g. —CH₃ and —CH₂—CH₃. In these radicals, C₁₋₂-alkyl represents C1- or C2-alkyl, C₁₋₃-alkyl represents C1-, C2- or C3-alkyl, C₁₋₄-alkyl represents C1-, C2-, C3- or C4-alkyl, C₁₋₅-alkyl represents C1-, C2-, C3-, C4-, or C5-alkyl, C₁₋₆-alkyl represents C1-, C2-, C3-, C4-, C5- or C6-alkyl, C₁₋₇-alkyl represents C1-, C2-, C3-, C4-, C5-, C6- or C7-alkyl, C₁₋₈-alkyl represents C1-, C2-, C3-, C4-, C5-, C6-, C7- or C8-alkyl, C₁₋₁₀-alkyl represents C1-, C2-, C3-, C4-, C5-, C6-, C7-, C8-, C9- or C10-alkyl and C₁₋₁₈-alkyl represents C1-, C2-, C3-, C4-, C5-, C6-, C7-, C8-, C9-, C10-, C11-, C12-, C13-, C14-, C15-, C16-, C17- or C18-alkyl. The alkyl radicals are preferably methyl, ethyl, propyl, methylethyl, butyl, 1-methylpropyl, 2-methylpropyl, 1,1-dimethylethyl, pentyl, 1,1-dimethylpropyl, 1,2-dimethylpropyl, 2,2-dimethylpropyl, hexyl, 1-methylpentyl, if substituted also CHF₂, CF₃ or CH₂OH etc. Preferably alkyl is understood in the context of this invention as C₁₋₈alkyl like methyl, ethyl, propyl, butyl, pentyl, hexyl, heptyl, or octyl; preferably is C₁₋₆alkyl like methyl, ethyl, propyl, butyl, pentyl, or hexyl; more preferably is C₁₋₄alkyl like methyl, ethyl, propyl or butyl.

Alkenyl is understood as meaning unsaturated, linear or branched hydrocarbons, which may be unsubstituted or substituted once or several times. It encompasses groups like e.g. —CH═CH—CH₃. The alkenyl radicals are preferably vinyl (ethenyl), allyl (2-propenyl). Preferably in the context of this invention alkenyl is C₂₋₁₀-alkenyl or C₂₋₈-alkenyl like ethylene, propylene, butylene, pentylene, hexylene, heptylene or octylene; or is C₂₋₆-alkenyl like ethylene, propylene, butylene, pentylene, or hexylene; or is C₂₋₄-alkenyl, like ethylene, propylene, or butylenes.

Alkynyl is understood as meaning unsaturated, linear or branched hydrocarbons, which may be unsubstituted or substituted once or several times. It encompasses groups like e.g. —C≡C—CH₃ (1-propinyl). Preferably alkynyl in the context of this invention is C₂₋₁₀-alkynyl or C₂₋₈-alkynyl like ethyne, propyne, butyene, pentyne, hexyne, heptyne, or octyne; or is C₂₋₆-alkynyl like ethyne, propyne, butyene, pentyne, or hexyne; or is C₂₋₄-alkynyl like ethyne, propyne, butyene, pentyne, or hexyne.

In connection with alkyl (also in alkylaryl, alkylheterocyclyl or alkylcycloalkyl), alkenyl, alkynyl and O-alkyl—unless defined otherwise—the term substituted in the context of this invention is understood as meaning replacement of at least one hydrogen radical on a carbon atom by halogen (F, Cl, Br, I), —NRₖR_{k′}, —SRₖ, —S(O)Rₖ, —S(O)₂Rₖ, —ORₖ, —C(O)Rₖ, —C(O)ORₖ, —CN, —C(O)NRₖR_{k′}, haloalkyl, haloalkoxy, being Rₖ represented by R₉, R₁₁, R₁₂, or R₁₃, (being R_{k′} represented by R_{9′}, R_{11′}, R_{12′}, or R_{13′}; being R_{k″} represented by R_{9″}, R_{11″}, R_{12″}, or R_{13″}; wherein R₁ to R_{14″}, and Rₓ and R_{x′}, and R_{c} and R_{c′} are as defined in the description, and wherein when different radicals R₁ to R_{14″}, and Rₓ and R_{x′} and R_{c} and R_{c′} are present simultaneously in Formula I they may be identical or different.

Most preferably in connection with alkyl (also in alkylaryl, alkylheterocyclyl or alkylcycloalkyl), alkenyl, alkynyl or O-alkyl, substituted is understood in the context of this invention that any alkyl (also in alkylaryl, alkylheterocyclyl or alkylcycloalkyl), alkenyl, alkynyl or O-alkyl which is substituted with one or more of halogen (F, Cl, Br, I), —NRₖR_{k′}, —ORₖ, —CN, —SRₖ, haloalkyl, haloalkoxy, being Rₖ represented by R₉, R₁₁, R₁₂, or R₁₃, (being R_{k′} represented by R_{9′}, R_{11′}, R_{12′}, or R_{13′}; being R_{k″} represented by R_{9″}, R_{11″}, R_{12″}, or R_{13″}; wherein R₁ to R_{14″}, and Rₓ and R_{x′} and R_{c} and R_{c′} are as defined in the description, and wherein when different radicals R₁ to R_{14″} and Rₓ and R_{x′} and R_{c} and R_{c′} are present simultaneously in Formula I they may be identical or different.

More than one replacement on the same molecule and also on the same carbon atom is possible with the same or different substituents. This includes for example 3 hydrogens being replaced on the same C atom, as in the case of CF₃, or at different places of the same molecule, as in the case of e.g. —CH(OH)—CH═CH—CHCl₂.

In the context of this invention haloalkyl is understood as meaning an alkyl being substituted once or several times by a halogen (selected from F, Cl, Br, I). It encompasses e.g. —CH₂Cl, —CH₂F, —CHCl₂, —CHF₂, —CCl₃, —CF₃ and —CH₂—CHCl₂. Preferably haloalkyl is understood in the context of this invention as halogen-substituted C₁₋₄-alkyl representing halogen substituted C1-, C2-, C3- or C4-alkyl. The halogen-substituted alkyl radicals are thus preferably methyl, ethyl, propyl, and butyl. Preferred examples include —CH₂Cl, —CH₂F, —CHCl₂, —CHF₂, and —CF₃.

In the context of this invention haloalkoxy is understood as meaning an —O-alkyl being substituted once or several times by a halogen (selected from F, Cl, Br, I). It encompasses e.g. —OCH₂Cl, —OCH₂F, —OCHCl₂, —OCHF₂, —OCCl₃, —OCF₃ and —OCH₂—CHCl₂. Preferably haloalkyl is understood in the context of this invention as halogen-substituted —OC₁₋₄-alkyl representing halogen substituted C1-, C2-, C3- or C4-alkoxy. The halogen-substituted alkyl radicals are thus preferably O-methyl, O-ethyl, O-propyl, and O-butyl. Preferred examples include —OCH₂Cl, —OCH₂F, —OCHCl₂, —OCHF₂, and —OCF₃.

In the context of this invention cycloalkyl is understood as meaning saturated and unsaturated (but not aromatic) cyclic hydrocarbons (without a heteroatom in the ring), which can be unsubstituted or once or several times substituted. Furthermore, C₃₋₄-cycloalkyl represents C3- or C4-cycloalkyl, C₃₋₅-cycloalkyl represents C3-, C4- or C5-cycloalkyl, C₃₋₆-cycloalkyl represents C3-, C4-, C5- or C6-cycloalkyl, C₃₋₇-cycloalkyl represents C3-, C4-, C5-, C6- or C7-cycloalkyl, C₃₋₈-cycloalkyl represents C3-, C4-, C5-, C6-, C7- or C8-cycloalkyl, C₄₋₅-cycloalkyl represents C4- or C5-cycloalkyl, C₄₋₆-cycloalkyl represents C4-, C5- or C6-cycloalkyl, C₄₋₇-cycloalkyl represents C4-, C5-, C6- or C7-cycloalkyl, C₅₋₆-cycloalkyl represents C5- or C6-cycloalkyl and C₅₋₇-cycloalkyl represents C5-, C6- or C7-cycloalkyl. Examples are cyclopropyl, 2-methylcyclopropyl, cyclopropylmethyl, cyclobutyl, cyclopentyl, cyclopentylmethyl, cyclohexyl, cycloheptyl, cyclooctyl, and also adamantyl. Preferably in the context of this invention cycloalkyl is C₃₋₈cycloalkyl like cyclopropyl, cyclobutyl, cyclopentyl, cyclohexyl, cycloheptyl, or cyclooctyl; or is C₃₋₇cycloalkyl like cyclopropyl, cyclobutyl, cyclopentyl, cyclohexyl, or cycloheptyl; or is C₃₋₆cycloalkyl like cyclopropyl, cyclobutyl, cyclopentyl or cyclohexyl, especially cyclopentyl or cyclohexyl.

Aryl is understood as meaning 5 to 18 membered mono or polycyclic ring systems with at least one aromatic ring but without heteroatoms even in only one of the rings. Examples are phenyl, naphthyl, fluoranthenyl, fluorenyl, tetralinyl or indanyl, 9H-fluorenyl or anthracenyl radicals, which can be unsubstituted or once or several times substituted. Most preferably aryl is understood in the context of this invention as phenyl, naphthyl or anthracenyl, preferably is phenyl.

A heterocyclyl radical or group (also called heterocyclyl hereinafter) is understood as meaning 5 to 18 membered mono or polycyclic heterocyclic ring systems, with at least one saturated or unsaturated ring which contains one or more heteroatoms selected from the group consisting of nitrogen, oxygen and/or sulfur in the ring. A heterocyclic group can also be substituted once or several times.

Examples include non-aromatic heterocyclyls such as tetrahydropyrane, oxazepane, morpholine, piperidine, pyrrolidine as well as heteroaryls such as furan, benzofuran, thiophene, benzothiophene, pyrrole, pyridine, pyrimidine, pyrazine, quinoline, isoquinoline, phthalazine, thiazole, benzothiazole, indole, benzotriazole, carbazole and quinazoline.

Subgroups inside the heterocyclyls as understood herein include heteroaryls and non-aromatic heterocyclyls.
the heteroaryl (being equivalent to heteroaromatic radicals or aromatic heterocyclyls) is an aromatic 5 to 18 membered mono or polycyclic heterocyclic ring system of one or more rings of which at least one aromatic ring contains one or more heteroatoms selected from the group consisting of nitrogen, oxygen and/or sulfur in the ring; preferably is a 5 to 18 membered mono or polycyclic aromatic heterocyclic ring system of one or two rings of which at least one aromatic ring contains one or more heteroatoms selected from the group consisting of nitrogen, oxygen and/or sulfur in the ring, more preferably is selected from furan, benzofuran, thiophene, benzothiophene, pyrrole, pyridine, pyrimidine, pyrazine, quinoline, isoquinoline, phthalazine, benzothiazole, indole, benzotriazole, carbazole, quinazoline, thiazole, imidazole, pyrazole, oxazole, thiophene and benzimidazole;
the non-aromatic heterocyclyl is a 5 to 18 membered mono or polycyclic heterocyclic ring system of one or more rings of which at least one ring—with this (or these) ring(s) then not being aromatic—contains one or more heteroatoms selected from the group consisting of nitrogen, oxygen and/or sulfur in the ring; preferably is a 5 to 18 membered mono or polycyclic heterocyclic ring system of one or two rings of which one or both rings—with this one or two rings then not being aromatic—contain/s one or more heteroatoms selected from the group consisting of nitrogen, oxygen and/or sulfur in the ring, more preferably is selected from oxazepam, pyrrolidine, piperidine, piperazine, tetrahydropyran, morpholine, indoline, oxopyrrolidine, benzodioxane, especially is benzodioxane, morpholine, tetrahydropyran, piperidine, oxopyrrolidine and pyrrolidine.

Preferably in the context of this invention heterocyclyl is defined as a 5 to 18 membered mono or polycyclic heterocyclic ring system of one or more saturated or unsaturated rings of which at least one ring contains one or more heteroatoms selected from the group consisting of nitrogen, oxygen and/or sulfur in the ring. Preferably it is a 5 to 18 membered mono or polycyclic heterocyclic ring system of one or two saturated or unsaturated rings of which at least one ring contains one or more heteroatoms selected from the group consisting of nitrogen, oxygen and/or sulfur in the ring.

Preferred examples of heterocyclyls include oxazepan, pyrrolidine, imidazole, oxadiazole, tetrazole, pyridine, pyrimidine, piperidine, piperazine, benzofuran, benzimidazole, indazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, tetrahydroisoquinoline, phthalazine, benzo-1,2,5-thiadiazole, indole, benzotriazole, benzoxazole oxopyrrolidine, pyrimidine, benzodioxolane, benzodioxane, carbazole and quinazoline, especially is pyridine, pyrazine, indazole, benzodioxane, thiazole, benzothiazole, morpholine, tetrahydropyrane, pyrazole, imidazole, piperidine, thiophene, indole, benzimidazole, pyrrolo[2,3b]pyridine, benzoxazole, oxopyrrolidine, pyrimidine, oxazepane and pyrrolidine.

In the context of this invention oxopyrrolidine is understood as meaning pyrrolidin-2-one.

In connection with aromatic heterocyclyls (heteroaryls), non-aromatic heterocyclyls, aryls and cycloalkyls, when a ring system falls within two or more of the above cycle definitions simultaneously, then the ring system is defined first as an aromatic heterocyclyl (heteroaryl) if at least one aromatic ring contains a heteroatom. If no aromatic ring contains a heteroatom, then the ring system is defined as a non-aromatic heterocyclyl if at least one non-aromatic ring contains a heteroatom. If no non-aromatic ring contains a heteroatom, then the ring system is defined as an aryl if it contains at least one aryl cycle. If no aryl is present, then the ring system is defined as a cycloalkyl if at least one non-aromatic cyclic hydrocarbon is present.

In the context of this invention alkylaryl is understood as meaning an aryl group (see above) being connected to another atom through a C₁₋₆-alkyl (see above) which may be branched or linear and is unsubstituted or substituted once or several times. Preferably alkylaryl is understood as meaning an aryl group (see above) being connected to another atom through 1 to 4 (—CH₂—) groups. Most preferably alkylaryl is benzyl (i.e. —CH₂-phenyl).

In the context of this invention alkylheterocyclyl is understood as meaning an heterocyclyl group being connected to another atom through a C₁₋₆-alkyl (see above) which may be branched or linear and is unsubstituted or substituted once or several times. Preferably alkylheterocyclyl is understood as meaning an heterocyclyl group (see above) being connected to another atom through 1 to 4 (—CH₂—) groups. Most preferably alkylheterocyclyl is —CH₂-pyridine.

In the context of this invention alkylcycloalkyl is understood as meaning an cycloalkyl group being connected to another atom through a C₁₋₆-alkyl (see above) which may be branched or linear and is unsubstituted or substituted once or several times. Preferably alkylcycloalkyl is understood as meaning an cycloalkyl group (see above) being connected to another atom through 1 to 4 (—CH₂—) groups. Most preferably alkylcycloalkyl is —CH₂-cyclopropyl.

Preferably, the aryl is a monocyclic aryl. More preferably the aryl is a 5, 6 or 7 membered monocyclic aryl. Even more preferably the aryl is a 5 or 6 membered monocyclic aryl.

Preferably, the heteroaryl is a monocyclic heteroaryl. More preferably the heteroaryl is a 5, 6 or 7 membered monocyclic heteroaryl. Even more preferably the heteroaryl is a 5 or 6 membered monocyclic heteroaryl.

Preferably, the non-aromatic heterocyclyl is a monocyclic non-aromatic heterocyclyl. More preferably the non-aromatic heterocyclyl is a 4, 5, 6 or 7 membered monocyclic non-aromatic heterocyclyl. Even more preferably the non-aromatic heterocyclyl is a 5 or 6 membered monocyclic non-aromatic heterocyclyl.

Preferably, the cycloalkyl is a monocyclic cycloalkyl. More preferably the cycloalkyl is a 3, 4, 5, 6, 7 or 8 membered monocyclic cycloalkyl. Even more preferably the cycloalkyl is a 3, 4, 5 or 6 membered monocyclic cycloalkyl.

In connection with aryl (including alkyl-aryl), cycloalkyl (including alkyl-cycloalkyl), or heterocyclyl (including alkyl-heterocyclyl), substituted is understood—unless defined otherwise—as meaning substitution of the ring-system of the aryl or alkyl-aryl, cycloalkyl or alkyl-cycloalkyl; heterocyclyl or alkyl-heterocyclyl with one or more of halogen (F, Cl, Br, I), —Rₖ, —ORₖ, —CN, —NO₂, —NRₖR_{k′}, —C(O)ORₖ, NRₖC(O)R_{k′}, —C(O)NRₖR_{k′}, —NRₖS(O)₂R_{k′}, ═O, —OCH₂CH₂OH, —NRₖC(O)NR_{k′}R_{k″}, —S(O)₂NRₖR_{k′}, —NRₖS(O)₂NR_{k′}R_{k″}, haloalkyl, haloalkoxy, —SRₖ, —S(O)Rₖ, —S(O)₂Rₖ or C(CH₃)ORₖ, or substituted or unsubstituted alkylcycloalkyl, substituted or unsubstituted alkylaryl, substituted or unsubstituted alkylheterocyclyl, with Rₖ, R_{k′} and R_{k″} independently being either H or a saturated or unsaturated, linear or branched, substituted or unsubstituted C₁₋₆-alkyl; a saturated or unsaturated, linear or branched, substituted or unsubstituted C₁₋₆-alkyl; a saturated or unsaturated, linear or branched, substituted or unsubstituted —O—C₁₋₆-alkyl (alkoxy); a saturated or unsaturated, linear or branched, substituted or unsubstituted —S—C₁₋₆-alkyl; a saturated or unsaturated, linear or branched, substituted or unsubstituted —C(O)—C₁₋₆-alkyl-group; a saturated or unsaturated, linear or branched, substituted or unsubstituted —C(O)—O—C₁₋₆-alkyl-group; a substituted or unsubstituted aryl or alkyl-aryl; a substituted or unsubstituted cycloalkyl or alkyl-cycloalkyl; a substituted or unsubstituted heterocyclyl or alkyl-heterocyclyl, being Rₖ one of R₁₀, R₁₁, R₁₂ or R₁₄, (being R_{k′} one of R_{10′}, R_{11′}, R_{12′} or R_{14′}, being R_{k″} one of R_{10″}, R_{11″}, R_{12″}, or R_{14″}; wherein R₁ to R_{14″} and Rₓ and R_{x′} and R_{c} and R_{c′} are as defined in the description, and wherein when different radicals R₁ to R_{14″} and Rₓ and R_{x′} and R_{c} and R_{c′} are present simultaneously in Formula I they may be identical or different.

Most preferably in connection with aryl (including alkyl-aryl), cycloalkyl (including alkyl-cycloalkyl), or heterocyclyl (including alkyl-heterocyclyl), substituted is understood in the context of this invention that any aryl, cycloalkyl and heterocyclyl which is substituted is substituted (also in an alyklaryl, alkylcycloalkyl or alkylheterocyclyl) with one or more of halogen (F, Cl, Br, I), —Rₖ, —ORₖ, —CN, —NO₂, —NRₖR_{k′″}, NRₖC(O)R_{k′}, —NRₖS(O)₂R_{k′}, —S(O)₂NRₖR_{k′}, —NRₖC(O)NR_{k′}R_{k″}, haloalkyl, haloalkoxy, —SRₖ, —S(O)Rₖ or S(O)₂Rₖ, or substituted or unsubstituted alkylcycloalkyl, substituted or unsubstituted alkylaryl, substituted or unsubstituted alkylheterocyclyl, being Rₖ one of R₁₀, R₁₁, R₁₂ or R₁₄, (being R_{k′} one of R_{10′}, R_{11′}, R_{12′} or R_{14′}; being R_{k″} one of R_{10″}, R_{11″}, R_{12″}, or R_{14″}; wherein R₁ to R_{14″}, and Rₓ and R_{x′}, and R_{c} and R_{c′} are as defined in the description, and wherein when different radicals R₁ to R_{14″}, and Rₓ and R_{x′} and R_{c} and R_{c′} are present simultaneously in Formula I they may be identical or different.

In connection with cycloalkyl (including alkyl-cycloalkyl), or heterocyclyl (including alkylheterocyclyl) namely non-aromatic heterocyclyl (including non-aromatic alkyl-heterocyclyl), substituted is also understood—unless defined otherwise—as meaning substitution of the ring-system of the cycloalkyl or alkyl-cycloalkyl; non-aromatic heterocyclyl or non aromatic alkyl-heterocyclyl with

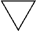

(leading to a spiro structure) or with ═O.

A ring system is a system consisting of at least one ring of connected atoms but including also systems in which two or more rings of connected atoms are joined with “joined” meaning that the respective rings are sharing one (like a spiro structure), two or more atoms being a member or members of both joined rings.

The term “leaving group” means a molecular fragment that departs with a pair of electrons in heterolytic bond cleavage. Leaving groups can be anions or neutral molecules. Common anionic leaving groups are halides such as Cl—, Br—, and I—, and sulfonate esters, such as tosylate (TsO—) or mesylate.

The term “salt” is to be understood as meaning any form of the active compound used according to the invention in which it assumes an ionic form or is charged and is coupled with a counter-ion (a cation or anion) or is in solution. By this are also to be understood complexes of the active compound with other molecules and ions, in particular complexes via ionic interactions.

The term “physiologically acceptable salt” means in the context of this invention any salt that is physiologically tolerated (most of the time meaning not being toxic-especially not caused by the counter-ion) if used appropriately for a treatment especially if used on or applied to humans and/or mammals.

These physiologically acceptable salts can be formed with cations or bases and in the context of this invention is understood as meaning salts of at least one of the compounds used according to the invention—usually a (deprotonated) acid—as an anion with at least one, preferably inorganic, cation which is physiologically tolerated—especially if used on humans and/or mammals. The salts of the alkali metals and alkaline earth metals are particularly preferred, and also those with NH₄, but in particular (mono)- or (di)sodium, (mono)- or (di)potassium, magnesium or calcium salts.

Physiologically acceptable salts can also be formed with anions or acids and in the context of this invention is understood as meaning salts of at least one of the compounds used according to the invention as the cation with at least one anion which are physiologically tolerated—especially if used on humans and/or mammals. By this is understood in particular, in the context of this invention, the salt formed with a physiologically tolerated acid, that is to say salts of the particular active compound with inorganic or organic acids which are physiologically tolerated—especially if used on humans and/or mammals. Examples of physiologically tolerated salts of particular acids are salts of: hydrochloric acid, hydrobromic acid, sulfuric acid, methanesulfonic acid, formic acid, acetic acid, oxalic acid, succinic acid, malic acid, tartaric acid, mandelic acid, fumaric acid, lactic acid or citric acid.

The compounds of the invention may be present in crystalline form or in the form of free compounds like a free base or acid.

Any compound that is a solvate of a compound according to the invention like a compound according to general formula I defined above is understood to be also covered by the scope of the invention. Methods of solvation are generally known within the art. Suitable solvates are pharmaceutically acceptable solvates. The term “solvate” according to this invention is to be understood as meaning any form of the active compound according to the invention in which this compound has attached to it via non-covalent binding another molecule (most likely a polar solvent). Especially preferred examples include hydrates and alcoholates, like methanolates or ethanolates.

Any compound that is a prodrug of a compound according to the invention like a compound according to general formula I defined above is understood to be also covered by the scope of the invention. The term “prodrug” is used in its broadest sense and encompasses those derivatives that are converted in vivo to the compounds of the invention. Such derivatives would readily occur to those skilled in the art, and include, depending on the functional groups present in the molecule and without limitation, the following derivatives of the present compounds: esters, amino acid esters, phosphate esters, metal salts sulfonate esters, carbamates, and amides. Examples of well known methods of producing a prodrug of a given acting compound are known to those skilled in the art and can be found e.g. in Krogsgaard-Larsen et al. “Textbook of Drug design and Discovery” Taylor & Francis (April 2002).

Any compound that is a N-oxide of a compound according to the invention like a compound according to general formula I defined above is understood to be also covered by the scope of the invention.

Unless otherwise stated, the compounds of the invention are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon or of a nitrogen by ¹⁵N-enriched nitrogen are within the scope of this invention. This would especially also apply to the provisos described above so that any mentioning of hydrogen or any “H” in a formula would also cover deuterium or tritium.

The compounds of formula (I) as well as their salts or solvates of the compounds are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I), or of its salts. This applies also to its solvates or prodrugs.

In a further embodiment the compound according to the invention of general Formula (I)

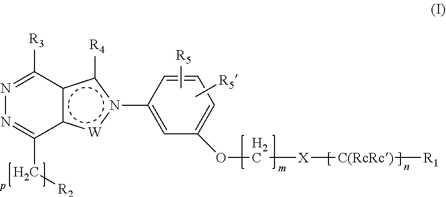

wherein
m is 0, 1, 2, 3 or 4;
n is 0, 1, 2, 3 or 4;
p is 0, 1, 2, 3 or 4;
W is nitrogen or —C(R_{4′})—;
X is selected from a bond, substituted or unsubstituted aryl or —C(RₓR_{x′})—;
Rₓ is selected from substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
Rₓ, is selected from hydrogen, substituted or unsubstituted C₁₋₆ alkyl, substituted or unsubstituted C₂₋₆ alkenyl and substituted or unsubstituted C₂₋₆ alkynyl;
   the alkyl, alkenyl or alkynyl in Rₓ or R_{x′}, if substituted, is substituted with one or more substituent/s selected from —OR₉, halogen, —CN, haloalkyl, haloalkoxy, and —NR₉R_{9′};
wherein R₉ and R_{9′}, are independently selected from hydrogen, unsubstituted C₁₋₆ alkyl, unsubstituted C₂₋₆ alkenyl, and unsubstituted C₂₋₆ alkynyl;
   the aryl, heterocyclyl or cycloalkyl, also in alkylaryl, alkylcycloalkyl and alkylheterocyclyl, in Rₓ, if substituted, is substituted with one or more substituent/s selected from halogen, —R₁₀, —OR₁₀, —NO₂, —NR₁₀R_{10′}, —NR₁₀C(O)R_{10′}, —NR₁₀S(O)₂R_{10′}, —S(O)₂NR₁₀R_{10′}, —NR₁₀C(O)NR_{10′}R_{10″}, —SR₁₀, —S(O)R₁₀, S(O)₂R₁₀, —CN, haloalkyl, haloalkoxy, —C(O)OR₁₀, —C(O)NR₁₀R_{10′}, —OCH₂CH₂OR₁₀, —NR₁₀S(O)₂NR_{10′}R_{10″}, and C(CH₃)₂OR₁₀;
wherein R₁₀, R_{10′} and R_{10″} are independently selected from hydrogen, unsubstituted C₁₋₆ alkyl, unsubstituted C₂₋₆ alkenyl, unsubstituted C₂₋₆ alkynyl, unsubstituted aryl, unsubstituted alkylaryl, unsubstituted cycloalkyl and unsubstituted alkylcycloalkyl, unsubstituted heterocyclyl and unsubstituted alkyheterocyclyl;
   R₁ is selected from —NR₆R_{6′}, and substituted or unsubstituted N-containing-heterocyclyl;
wherein R₆ and R_{6′}, are independently selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
wherein said cycloalkyl, aryl or heterocyclyl in R₁ or R₆, also in alkylaryl, alkylcycloalkyl and alkylheterocyclyl, if substituted, is substituted with one or more substituent/s selected from halogen, —R₁₁, —OR₁₁, —NO₂, —NR₁₁R_{11′}, NR₁₁C(O)R_{11′}, —NR₁₁S(O)₂R_{11′}, —S(O)₂NR₁₁R_{11′}, —NR₁₁C(O)NR_{11′}R_{11″}, —SR₁₁, —S(O)R₁₁, S(O)₂R₁₁, —CN, haloalkyl, haloalkoxy, —C(O)OR₁₁, —C(O)NR₁₁R_{11′}, —OCH₂CH₂OH, —NR₁₁S(O)₂NR_{11′}R_{11″}, C(CH₃)₂OR₁₁, substituted or unsubstituted alkylaryl, substituted or unsubstituted alkylcycloalkyl and substituted or unsubstituted alkyheterocyclyl;
wherein the alkyl, alkenyl or alkynyl in R₆ or R_{6′}, if substituted, is substituted with one or more substituent/s selected from —OR₁₁, halogen, —CN, haloalkyl, haloalkoxy and —NR₁₁R_{11′};
wherein R₁₁, R_{11′} and R_{11″}, are independently selected from hydrogen, unsubstituted C₁₋₆ alkyl, unsubstituted C₂₋₆ alkenyl and unsubstituted C₂₋₆ alkynyl;
   R₂ is selected from hydrogen, —NR₇R_{7′}, —CN, —CHR₇R_{7′} and substituted or unsubstituted heterocyclyl;
wherein R₇ and R_{7′} are independently selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
wherein said cycloalkyl, aryl or heterocyclyl in R₂, R₇ or R_{7′}, also in alkylaryl, alkylcycloalkyl and alkylheterocyclyl, if substituted, is substituted with one or more substituent/s selected from halogen, —R₁₂, —OR₁₂, —NO₂, —NR₁₂R_{12′}, NR₁₂C(O)R_{12′}, —NR₁₂S(O)₂R_{12′}, —S(O)₂NR₁₂R_{12′}, —NR₁₂C(O)NR_{12′}R_{12″}, —SR₁₂, —S(O)R₁₂, S(O)₂R₁₂, —CN, haloalkyl, haloalkoxy, —C(O)OR₁₂, —C(O)NR₁₂R_{12′}, —OCH₂CH₂OR₁₂, NR₁₂S(O)₂NR_{12′}R_{12″} and —C(CH₃)₂OR₁₂;
wherein the alkyl, alkenyl or alkynyl in R₇ or R_{7′}, if substituted, is substituted with one or more substituent/s selected from —OR₁₂, halogen, —CN, haloalkyl, haloalkoxy and —NR₁₂R_{12′};
wherein R₁₂, R_{12′} and R_{12″} are independently selected from hydrogen, unsubstituted C₁₋₆ alkyl, and unsubstituted C₂₋₆ alkenyl and unsubstituted C₂₋₆ alkynyl;
   R₃ is selected from hydrogen, substituted or unsubstituted C₁₋₆ alkyl, substituted or unsubstituted C₂₋₆ alkenyl and substituted or unsubstituted C₂₋₆ alkynyl;
   R₄ is selected from hydrogen, substituted or unsubstituted C₁₋₆ alkyl, substituted or unsubstituted C₂₋₆ alkenyl and substituted or unsubstituted C₂₋₆ alkynyl;
   R_{4′} is selected from hydrogen, substituted or unsubstituted C₁₋₆ alkyl, substituted or unsubstituted C₂₋₆ alkenyl and substituted or unsubstituted C₂₋₆ alkynyl;
   R₅ and R_{5′} are independently selected from hydrogen, halogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl, substituted or unsubstituted alkyheterocyclyl, —OR₈, —NO₂, —NR₈R_{8′}, —NR₈C(O)R_{8′}, —NR₈S(O)₂R_{8′}, —S(O)₂NR₈R_{8′}, —NR₈C(O)NR_{8′}R_{8″}, —SR₈, —S(O)R₈, S(O)₂R₈, —CN, haloalkyl, haloalkoxy, —C(O)OR₈, —C(O)NR₈R_{8′}, —OCH₂CH₂OR₈, —NR₈S(O)₂NR_{8′}R_{8″}, and C(CH₃)₂OR₈;
wherein R₈, R_{8′} and R_{8″} are independently selected from hydrogen, unsubstituted C₁₋₆ alkyl, unsubstituted C₂₋₆ alkenyl, and unsubstituted C₂₋₆ alkynyl;
   R_{c} and R_{c′} are independently selected from hydrogen, substituted or unsubstituted C₁₋₆ alkyl, substituted or unsubstituted C₂₋₆ alkenyl and substituted or unsubstituted C₂₋₆ alkynyl;
   alternatively, R_{c} and R_{c′} may form with the carbon atom to which they are attached, a substituted or unsubstituted cycloalkyl;
   the alkyl, alkenyl or alkynyl, other than those defined in Rₓ, R_{x′}, R₆, R_{6′}, R₇ or R_{7′}, if substituted, is substituted with one or more substituent/s selected from —OR₁₃, halogen, —CN, haloalkyl, haloalkoxy and —NR₁₃R_{13′};
wherein R₁₃ and R_{13′} are independently selected from hydrogen, unsubstituted C₁₋₆ alkyl, unsubstituted C₂₋₆ alkenyl, and unsubstituted C₂₋₆ alkynyl;
   the aryl, heterocyclyl or cycloalkyl, also in alkylaryl, alkylcycloalkyl and alkylheterocyclyl, other than those defined in Rₓ, R_{x′}, R₁, R₂, R₆, R_{6′}, R₇ or R_{7′}, if substituted, is substituted with one or more substituent/s selected from halogen, —R₁₄, —OR₁₄, —NO₂, —NR₁₄R_{14′}, NR₁₄C(O)R_{14′}, —NR₁₄S(O)₂R_{14′}, —S(O)₂NR₁₄R_{14′}, —NR₁₄C(O)NR_{14′}R_{14″}, —SR₁₄, —S(O)R₁₄, S(O)₂R₁₄, —CN, haloalkyl, haloalkoxy, —C(O)OR₁₄, —C(O)NR₁₄R_{14′}, —OCH₂CH₂OR₁₄, —NR₁₄S(O)₂NR_{14′}R_{14″} and C(CH₃)₂OR₁₄;
wherein R₁₄, R_{14′} and R_{14″} are independently selected from hydrogen, unsubstituted C₁₋₆ alkyl, unsubstituted C₂₋₆ alkenyl, unsubstituted C₂₋₆ alkynyl, unsubstituted aryl, unsubstituted alkylaryl, unsubstituted cycloalkyl and unsubstituted alkylcycloalkyl, unsubstituted heterocyclyl and unsubstituted alkyheterocyclyl;

These preferred compounds according to the invention are optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I′)

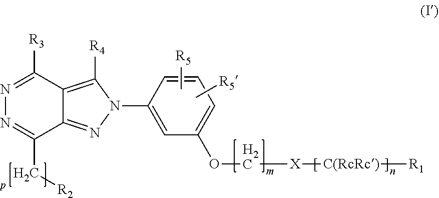

wherein
m is 0, 1, 2, 3 or 4;
n is 0, 1, 2, 3 or 4;
p is 0, 1, 2, 3 or 4;
X is selected from a bond, substituted or unsubstituted aryl or —C(RₓR_{x′})—;
Rₓ is selected from substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
Rₓ, is selected from hydrogen, substituted or unsubstituted C₁₋₆ alkyl, substituted or unsubstituted C₂₋₆ alkenyl and substituted or unsubstituted C₂₋₆ alkynyl;
   the alkyl, alkenyl or alkynyl in Rₓ or R_{x′}, if substituted, is substituted with one or more substituent/s selected from —OR₉, halogen, —CN, haloalkyl, haloalkoxy, and —NR₉R_{9′};
wherein R₉ and R_{9′} are independently selected from hydrogen, unsubstituted C₁₋₆ alkyl, unsubstituted C₂₋₆ alkenyl, and unsubstituted C₂₋₆ alkynyl;
   the aryl, heterocyclyl or cycloalkyl, also in alkylaryl, alkylcycloalkyl and alkylheterocyclyl, in Rₓ, if substituted, is substituted with one or more substituent/s selected from halogen, —R₁₀, —OR₁₀, —NO₂, —NR₁₀R_{10′}, —NR₁₀C(O)R_{10′}, —NR₁₀S(O)₂R_{10′}, —S(O)₂NR₁₀R_{10′}, —NR₁₀C(O)NR_{10′}R_{10″}, —SR₁₀, —S(O)R₁₀, S(O)₂R₁₀, —CN, haloalkyl, haloalkoxy, —C(O)OR₁₀, —C(O)NR₁₀R_{10′}, —OCH₂CH₂OR₁₀, —NR₁₀S(O)₂NR_{10′}R_{10″} and C(CH₃)₂OR₁₀;
wherein R₁₀, R_{10′} and R_{10″} are independently selected from hydrogen, unsubstituted C₁₋₆ alkyl, unsubstituted C₂₋₆ alkenyl, unsubstituted C₂₋₆ alkynyl, unsubstituted aryl, unsubstituted alkylaryl, unsubstituted cycloalkyl and unsubstituted alkylcycloalkyl, unsubstituted heterocyclyl and unsubstituted alkyheterocyclyl;
   R₁ is selected from —NR₆R_{6′}, and substituted or unsubstituted N-containing-heterocyclyl;
wherein R₆ and R_{6′}, are independently selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
wherein said cycloalkyl, aryl or heterocyclyl in R₁ or R₆, also in alkylaryl, alkylcycloalkyl and alkylheterocyclyl, if substituted, is substituted with one or more substituent/s selected from halogen, —R₁₁, —OR₁₁, —NO₂, —NR₁₁R_{11′}, NR₁₁C(O)R_{11′}, —NR₁₁S(O)₂R_{11′}, —S(O)₂NR₁₁R_{11′}, —NR₁₁C(O)NR_{11′}R_{11″}, —SR₁₁, —S(O)R₁₁, S(O)₂R₁₁, —CN, haloalkyl, haloalkoxy, —C(O)OR₁₁, —C(O)NR₁₁R_{11′}, —OCH₂CH₂OH, —NR₁₁S(O)₂NR_{11′}R_{11″}, C(CH₃)₂OR₁₁, substituted or unsubstituted alkylaryl, substituted or unsubstituted alkylcycloalkyl and substituted or unsubstituted alkyheterocyclyl;
wherein the alkyl, alkenyl or alkynyl in R₆ or R_{6′}, if substituted, is substituted with one or more substituent/s selected from —OR₁₁, halogen, —CN, haloalkyl, haloalkoxy and —NR₁₁R_{11′};
wherein R₁₁, R_{11′} and R_{11″} are independently selected from hydrogen, unsubstituted C₁₋₆ alkyl, unsubstituted C₂₋₆ alkenyl and unsubstituted C₂₋₆ alkynyl;
   R₂ is selected from hydrogen, —NR₇R_{7′}, —CN, —CHR₇R_{7′} and substituted or unsubstituted heterocyclyl;
wherein R₇ and R_{7′} are independently selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
wherein said cycloalkyl, aryl or heterocyclyl in R₂, R₇ or R_{7′}, also in alkylaryl, alkylcycloalkyl and alkylheterocyclyl, if substituted, is substituted with one or more substituent/s selected from halogen, —R₁₂, —OR₁₂, —NO₂, —NR₁₂R_{12′}, NR₁₂C(O)R_{12′}, —NR₁₂S(O)₂R_{12′}, —S(O)₂NR₁₂R_{12′}, —NR₁₂C(O)NR_{12′}R_{12″}, —SR₁₂, —S(O)R₁₂, S(O)₂R₁₂, —CN, haloalkyl, haloalkoxy, —C(O)OR₁₂, —C(O)NR₁₂R_{12′}, —OCH₂CH₂OR₁₂, NR₁₂S(O)₂NR_{12′}R_{12″} and —C(CH₃)₂OR₁₂;
wherein the alkyl, alkenyl or alkynyl in R₇ or R_{7′}, if substituted, is substituted with one or more substituent/s selected from —OR₁₂, halogen, —CN, haloalkyl, haloalkoxy and —NR₁₂R_{12′};
wherein R₁₂, R_{12′} and R_{12″} are independently selected from hydrogen, unsubstituted C₁₋₆ alkyl, and unsubstituted C₂₋₆ alkenyl and unsubstituted C₂₋₆ alkynyl;
   R₃ is selected from hydrogen, substituted or unsubstituted C₁₋₆ alkyl, substituted or unsubstituted C₂₋₆ alkenyl and substituted or unsubstituted C₂₋₆ alkynyl;
   R₄ is selected from hydrogen, substituted or unsubstituted C₁₋₆ alkyl, substituted or unsubstituted C₂₋₆ alkenyl and substituted or unsubstituted C₂₋₆ alkynyl;
   R_{4′} is selected from hydrogen, substituted or unsubstituted C₁₋₆ alkyl, substituted or unsubstituted C₂₋₆ alkenyl and substituted or unsubstituted C₂₋₆ alkynyl;
   R₅ and R_{5′} are independently selected from hydrogen, halogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl, substituted or unsubstituted alkyheterocyclyl, —OR₈, —NO₂, —NR₈R_{8′}, —NR₈C(O)R₈, —NR₈S(O)₂R_{8′}, —S(O)₂NR₈R_{8′}, —NR₈C(O)NR_{8′}R_{8″}, —SR₈, —S(O)R₈, S(O)₂R₈, —CN, haloalkyl, haloalkoxy, —C(O)OR₈, —C(O)NR₈R_{8′}, —OCH₂CH₂OR₈, —NR₈S(O)₂NR_{8′}R_{8″}, and C(CH₃)₂OR₈;
wherein R₈, R_{8′}, and R_{8″} are independently selected from hydrogen, unsubstituted C₁₋₆ alkyl, unsubstituted C₂₋₆ alkenyl, and unsubstituted C₂₋₆ alkynyl;
   R_{c} and R_{c′} are independently selected from hydrogen, substituted or unsubstituted C₁₋₆ alkyl, substituted or unsubstituted C₂₋₆ alkenyl and substituted or unsubstituted C₂₋₆ alkynyl;
   alternatively, R_{c} and R_{c′} may form with the carbon atom to which they are attached, a substituted or unsubstituted cycloalkyl;
   the alkyl, alkenyl or alkynyl, other than those defined in Rₓ, R_{x′}, R₆, R_{6′}, R₇ or R_{7′}, if substituted, is substituted with one or more substituent/s selected from —OR₁₃, halogen, —CN, haloalkyl, haloalkoxy and —NR₁₃R_{13′};
wherein R₁₃ and R_{13′} are independently selected from hydrogen, unsubstituted C₁₋₆ alkyl, unsubstituted C₂₋₆ alkenyl, and unsubstituted C₂₋₆ alkynyl;
   the aryl, heterocyclyl or cycloalkyl, also in alkylaryl, alkylcycloalkyl and alkylheterocyclyl, other than those defined in Rₓ, R_{x′}, R₁, R₂, R₆, R_{6′}, R₇ or R_{7′}, if substituted, is substituted with one or more substituent/s selected from halogen, —R₁₄, —OR₁₄, —NO₂, —NR₁₄R_{14′}, NR₁₄C(O)R_{14′}, —NR₁₄S(O)₂R_{14′}, —S(O)₂NR₁₄R_{14′}, —NR₁₄C(O)NR_{14′}R_{14″}, —SR₁₄, —S(O)R₁₄, S(O)₂R₁₄, —CN, haloalkyl, haloalkoxy, —C(O)OR₁₄, —C(O)NR₁₄R_{14′}, —OCH₂CH₂OR₁₄, —NR₁₄S(O)₂NR_{14′}R_{14″} and C(CH₃)₂OR₁₄;
wherein R₁₄, R_{14′} and R_{14″} are independently selected from hydrogen, unsubstituted C₁₋₆ alkyl, unsubstituted C₂₋₆ alkenyl, unsubstituted C₂₋₆ alkynyl, unsubstituted aryl, unsubstituted alkylaryl, unsubstituted cycloalkyl and unsubstituted alkylcycloalkyl, unsubstituted heterocyclyl and unsubstituted alkyheterocyclyl;

These preferred compounds according to the invention are optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I²′)

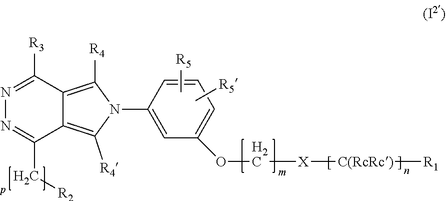

wherein
m is 0, 1, 2, 3 or 4;
n is 0, 1, 2, 3 or 4;
p is 0, 1, 2, 3 or 4;
X is selected from a bond, substituted or unsubstituted aryl or —C(RₓR_{x′})—;
Rₓ is selected from substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
R_{x′} is selected from hydrogen, substituted or unsubstituted C₁₋₆ alkyl, substituted or unsubstituted C₂₋₆ alkenyl and substituted or unsubstituted C₂₋₆ alkynyl;
   the alkyl, alkenyl or alkynyl in Rₓ or R_{x′}, if substituted, is substituted with one or more substituent/s selected from —OR₉, halogen, —CN, haloalkyl, haloalkoxy, and —NR₉R_{9′};
wherein R₉ and R_{9′} are independently selected from hydrogen, unsubstituted C₁₋₆ alkyl, unsubstituted C₂₋₆ alkenyl, and unsubstituted C₂₋₆ alkynyl;
   the aryl, heterocyclyl or cycloalkyl, also in alkylaryl, alkylcycloalkyl and alkylheterocyclyl, in Rₓ, if substituted, is substituted with one or more substituent/s selected from halogen, —R₁₀, —OR₁₀, —NO₂, —NR₁₀R_{10′}, —NR₁₀C(O)R_{10′}, —NR₁₀S(O)₂R_{10′}, —S(O)₂NR₁₀R_{10′}, —NR₁₀C(O)NR_{10′}R_{10″}, —SR₁₀, —S(O)R₁₀, S(O)₂R₁₀, —CN, haloalkyl, haloalkoxy, —C(O)OR₁₀, —C(O)NR₁₀R_{10′}, —OCH₂CH₂OR₁₀, —NR₁₀S(O)₂NR_{10′}R_{10″} and C(CH₃)₂OR₁₀;
wherein R₁₀, R_{10′} and R_{10″} are independently selected from hydrogen, unsubstituted C₁₋₆ alkyl, unsubstituted C₂₋₆ alkenyl, unsubstituted C₂₋₆ alkynyl, unsubstituted aryl, unsubstituted alkylaryl, unsubstituted cycloalkyl and unsubstituted alkylcycloalkyl, unsubstituted heterocyclyl and unsubstituted alkyheterocyclyl;
   R₁ is selected from —NR₆R_{6′}, and substituted or unsubstituted N-containing-heterocyclyl;
wherein R₆ and R_{6′}, are independently selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
wherein said cycloalkyl, aryl or heterocyclyl in R₁ or R₆, also in alkylaryl, alkylcycloalkyl and alkylheterocyclyl, if substituted, is substituted with one or more substituent/s selected from halogen, —R₁₁, —OR₁₁, —NO₂, —NR₁₁R_{11′}, NR₁₁C(O)R_{11′}, —NR₁₁S(O)₂R_{11′}, —S(O)₂NR₁₁R_{11′}, —NR₁₁C(O)NR_{11′}R_{11″}, —SR₁₁, —S(O)R₁₁, S(O)₂R₁₁, —CN, haloalkyl, haloalkoxy, —C(O)OR₁₁, —C(O)NR₁₁R_{11′}, —OCH₂CH₂OH, —NR₁₁S(O)₂NR_{11′}R_{11″}, C(CH₃)₂OR₁₁, substituted or unsubstituted alkylaryl, substituted or unsubstituted alkylcycloalkyl and substituted or unsubstituted alkyheterocyclyl;
wherein the alkyl, alkenyl or alkynyl in R₆ or R_{6′}, if substituted, is substituted with one or more substituent/s selected from —OR₁₁, halogen, —CN, haloalkyl, haloalkoxy and —NR₁₁R_{11′};
wherein R₁₁, R_{11′} and R_{11″} are independently selected from hydrogen, unsubstituted C₁₋₆ alkyl, unsubstituted C₂₋₆ alkenyl and unsubstituted C₂₋₆ alkynyl;
   R₂ is selected from hydrogen, —NR₇R_{7′}, —CN, —CHR₇R_{7′} and substituted or unsubstituted heterocyclyl;
wherein R₇ and R_{7′} are independently selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
wherein said cycloalkyl, aryl or heterocyclyl in R₂, R₇ or R_{7′}, also in alkylaryl, alkylcycloalkyl and alkylheterocyclyl, if substituted, is substituted with one or more substituent/s selected from halogen, —R₁₂, —OR₁₂, —NO₂, —NR₁₂R_{12′}, NR₁₂C(O)R_{12′}, —NR₁₂S(O)₂R_{12′}, —S(O)₂NR₁₂R_{12′}, —NR₁₂C(O)NR_{12′}R_{12″}, —SR₁₂, —S(O)R₁₂, S(O)₂R₁₂, —CN, haloalkyl, haloalkoxy, —C(O)OR₁₂, —C(O)NR₁₂R_{12′}, —OCH₂CH₂OR₁₂, NR₁₂S(O)₂NR_{12′}R_{12″} and —C(CH₃)₂OR₁₂;
wherein the alkyl, alkenyl or alkynyl in R₇ or R_{7′}, if substituted, is substituted with one or more substituent/s selected from —OR₁₂, halogen, —CN, haloalkyl, haloalkoxy and —NR₁₂R_{12′};
wherein R₁₂, R_{12′} and R_{12″} are independently selected from hydrogen, unsubstituted C₁₋₆ alkyl, and unsubstituted C₂₋₆ alkenyl and unsubstituted C₂₋₆ alkynyl;
   R₃ is selected from hydrogen, substituted or unsubstituted C₁₋₆ alkyl, substituted or unsubstituted C₂₋₆ alkenyl and substituted or unsubstituted C₂₋₆ alkynyl;
   R₄ is selected from hydrogen, substituted or unsubstituted C₁₋₆ alkyl, substituted or unsubstituted C₂₋₆ alkenyl and substituted or unsubstituted C₂₋₆ alkynyl;
   R_{4′} is selected from hydrogen, substituted or unsubstituted C₁₋₆ alkyl, substituted or unsubstituted C₂₋₆ alkenyl and substituted or unsubstituted C₂₋₆ alkynyl;
   R₅ and R_{5′} are independently selected from hydrogen, halogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl, substituted or unsubstituted alkyheterocyclyl, —OR₈, —NO₂, —NR₈R_{8′}, —NR₈C(O)R_{8′}, —NR₈S(O)₂R_{8′}, —S(O)₂NR₈R_{8′}, —NR₈C(O)NR_{8′}R_{8″}, —SR₈, —S(O)R₈, S(O)₂R₈, —CN, haloalkyl, haloalkoxy, —C(O)OR₈, —C(O)NR₈R_{8′}, —OCH₂CH₂OR₈, —NR₈S(O)₂NR_{8′}R_{8″} and C(CH₃)₂OR₈;
wherein R₈, R_{8′}, and R_{8″} are independently selected from hydrogen, unsubstituted C₁₋₆ alkyl, unsubstituted C₂₋₆ alkenyl, and unsubstituted C₂₋₆ alkynyl;
   R_{c} and R_{c′} are independently selected from hydrogen, substituted or unsubstituted C₁₋₆ alkyl, substituted or unsubstituted C₂₋₆ alkenyl and substituted or unsubstituted C₂₋₆ alkynyl;
   alternatively, R_{c} and R_{c′} may form with the carbon atom to which they are attached, a substituted or unsubstituted cycloalkyl;
   the alkyl, alkenyl or alkynyl, other than those defined in Rₓ, R_{x′}, R₆, R_{6′}, R₇ or R_{7′}, if substituted, is substituted with one or more substituent/s selected from —OR₁₃, halogen, —CN, haloalkyl, haloalkoxy and —NR₁₃R_{13′};
wherein R₁₃ and R_{13′} are independently selected from hydrogen, unsubstituted C₁₋₆ alkyl, unsubstituted C₂₋₆ alkenyl, and unsubstituted C₂₋₆ alkynyl;
   the aryl, heterocyclyl or cycloalkyl, also in alkylaryl, alkylcycloalkyl and alkylheterocyclyl, other than those defined in Rₓ, R_{x′}, R₁, R₂, R₆, R_{6′}, R₇ or R_{7′}, if substituted, is substituted with one or more substituent/s selected from halogen, —R₁₄, —OR₁₄, —NO₂, —NR₁₄R_{14′}, NR₁₄C(O)R_{14′}, —NR₁₄S(O)₂R_{14′}, —S(O)₂NR₁₄R_{14′}, —NR₁₄C(O)NR_{14′}R_{14″}, —SR₁₄, —S(O)R₁₄, S(O)₂R₁₄, —CN, haloalkyl, haloalkoxy, —C(O)OR₁₄, —C(O)NR₁₄R_{14′}, —OCH₂CH₂OR₁₄, —NR₁₄S(O)₂NR_{14′}R_{14″} and C(CH₃)₂OR₁₄;
wherein R₁₄, R_{14′} and R_{14″} are independently selected from hydrogen, unsubstituted C₁₋₆ alkyl, unsubstituted C₂₋₆ alkenyl, unsubstituted C₂₋₆ alkynyl, unsubstituted aryl, unsubstituted alkylaryl, unsubstituted cycloalkyl and unsubstituted alkylcycloalkyl, unsubstituted heterocyclyl and unsubstituted alkyheterocyclyl;

These preferred compounds according to the invention are optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

m is 0, 1, 2, 3 or 4;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

m is 0, 1 or 2;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

n is 0, 1, 2, 3 or 4;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

n is 0, 1, 2 or 3;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

p is 0, 1, 2, 3 or 4;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

p is 0;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

W is nitrogen or —C(R_{4′})—;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

W is nitrogen;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

X is selected from a bond, substituted or unsubstituted aryl or —C(RₓR_{x′})—;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

X is a bond;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

X is —C(RₓR_{x′})—;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein
Rₓ is selected from substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein
Rₓ is selected from substituted or unsubstituted C₁₋₆ alkyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein
Rₓ is selected from substituted or unsubstituted aryl, substituted or unsubstituted alkylaryl and substituted or unsubstituted heterocyclyl
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein
R_{x′} is selected from hydrogen, substituted or unsubstituted C₁₋₆ alkyl, substituted or unsubstituted C₂₋₆ alkenyl and substituted or unsubstituted C₂₋₆ alkynyl;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein
R_{x′} is selected from hydrogen and substituted or unsubstituted C₁₋₆ alkyl;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

Rₓₐ is independently selected from hydrogen, halogen, —OR₁₀, —CN, haloalkoxy, and —C(O)NR₁₀R_{10′};
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁ is selected from —NR₆R_{6′} and substituted or unsubstituted N-containing-heterocyclyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₂ is selected from hydrogen, —NR₇R_{7′}, —CN, —CHR₇R_{7′} and substituted or unsubstituted heterocyclyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₂ is selected from —NR₇R_{7′} and substituted or unsubstituted heterocyclyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₃ is selected from hydrogen, substituted or unsubstituted C₁₋₆ alkyl, substituted or unsubstituted C₂₋₆ alkenyl and substituted or unsubstituted C₂₋₆ alkynyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₃ is selected from hydrogen and substituted or unsubstituted C₁₋₆ alkyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₃ is substituted or unsubstituted C₁₋₆ alkyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₄ is selected from hydrogen, substituted or unsubstituted C₁₋₆ alkyl, substituted or unsubstituted C₂₋₆ alkenyl and substituted or unsubstituted C₂₋₆ alkynyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₄ is selected from hydrogen and substituted or unsubstituted C₁₋₆ alkyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₄ is substituted or unsubstituted C₁₋₆ alkyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R_{4′} is selected from hydrogen, substituted or unsubstituted C₁₋₆ alkyl, substituted or unsubstituted C₂₋₆ alkenyl and substituted or unsubstituted C₂₋₆ alkynyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R_{4′} is selected from hydrogen and substituted or unsubstituted C₁₋₆ alkyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₅ and R_{5′} are independently selected from hydrogen, halogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl, substituted or unsubstituted alkyheterocyclyl, —OR₈, —NO₂, —NR₈R_{8′}, —NR₈C(O)R_{8′}, —NR₈S(O)₂R_{8′}, —S(O)₂NR₈R_{8′}, —NR₈C(O)NR_{8′}R_{8″}, —SR₈, —S(O)R₈, S(O)₂R₈, —CN, haloalkyl, haloalkoxy, —C(O)OR₈, —C(O)NR₈R_{8′}, —OCH₂CH₂OR₈, —NR₈S(O)₂NR_{8′}R_{8″}, and C(CH₃)₂OR₈;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₅ and R_{5′} are independently selected from hydrogen, halogen, substituted or unsubstituted C₁₋₆ alkyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl, substituted or unsubstituted alkyheterocyclyl, —OR₈, —NO₂, —NR₈R_{8′}, —NR₈C(O)R_{8′}, —NR₈S(O)₂R_{8′}, —S(O)₂NR₈R_{8′}, —NR₈C(O)NR_{8′}R_{8″}, —SR₈, —S(O)R₈, S(O)₂R₈, —CN, haloalkyl, haloalkoxy, —C(O)OR₈, —C(O)NR₈R_{8′}, —OCH₂CH₂OR₈, —NR₈S(O)₂NR_{8′}R_{8″}, and C(CH₃)₂OR₈;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R_{c} and R_{c′} are independently selected from hydrogen, substituted or unsubstituted C₁₋₆ alkyl, substituted or unsubstituted C₂₋₆ alkenyl and substituted or unsubstituted C₂₋₆ alkynyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R_{c} and R_{c′} are independently selected from hydrogen and substituted or unsubstituted C₁₋₆ alkyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R_{c} and R_{c′} may form with the carbon atom to which they are attached, a substituted or unsubstituted cycloalkyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₆ and R_{6′} are independently selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₆ and R_{6′} are independently selected from hydrogen, substituted or unsubstituted C₁₋₆ alkyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₆ and R_{6′} are independently selected from hydrogen, substituted or unsubstituted C₁₋₆ alkyl and substituted or unsubstituted alkylaryl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₆ₐ is selected from hydrogen, substituted or unsubstituted C₁₋₆ alkyl and substituted or unsubstituted alkylaryl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₆ₐ is substituted or unsubstituted alkylaryl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₇ and R_{7′} are independently selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl; optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₇ and R_{7′} are independently selected from hydrogen, substituted or unsubstituted C₁₋₆ alkyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₇ and R_{7′} are independently selected from hydrogen and substituted or unsubstituted C₁₋₆ alkyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₈, R_{8′} and R_{8″} are independently selected from hydrogen, unsubstituted C₁₋₆ alkyl, unsubstituted C₂₋₆ alkenyl, and unsubstituted C₂₋₆ alkynyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₈, R_{8′} and R_{8″} are independently selected from hydrogen and unsubstituted C₁₋₆ alkyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₉ and R_{9′} are independently selected from hydrogen, unsubstituted C₁₋₆ alkyl, unsubstituted C₂₋₆ alkenyl, and unsubstituted C₂₋₆ alkynyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₉ and R_{9′} are independently selected from hydrogen and unsubstituted C₁₋₆ alkyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₁₀, R_{10′} and R_{10″} are independently selected from hydrogen, unsubstituted C₁₋₆ alkyl, unsubstituted C₂₋₆ alkenyl, unsubstituted C₂₋₆ alkynyl, unsubstituted aryl, unsubstituted alkylaryl, unsubstituted cycloalkyl and unsubstituted alkylcycloalkyl, unsubstituted heterocyclyl and unsubstituted alkyheterocyclyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₁₀, R_{10′} and R_{10″} are independently selected from hydrogen, unsubstituted C₁₋₆ alkyl, unsubstituted aryl, unsubstituted alkylaryl, unsubstituted cycloalkyl and unsubstituted alkylcycloalkyl, unsubstituted heterocyclyl and unsubstituted alkyheterocyclyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₁₀, R_{10′} and R_{10″} are independently selected from hydrogen and unsubstituted C₁₋₆ alkyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₁₁, R_{11′} and R_{11″} are independently selected from hydrogen, unsubstituted C₁₋₆ alkyl, unsubstituted C₂₋₆ alkenyl and unsubstituted C₂₋₆ alkynyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₁₁, R_{11′} and R_{11″} are independently selected from hydrogen and unsubstituted C₁₋₆ alkyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₁₂, R_{12′} and R_{12″} are independently selected from hydrogen, unsubstituted C₁₋₆ alkyl, and unsubstituted C₂₋₆ alkenyl and unsubstituted C₂₋₆ alkynyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₁₂, R_{12′} and R_{12″} are independently selected from hydrogen and unsubstituted C₁₋₆ alkyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₁₃ and R_{13′} are independently selected from hydrogen, unsubstituted C₁₋₆ alkyl, unsubstituted C₂₋₆ alkenyl, and unsubstituted C₂₋₆ alkynyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₁₃ and R_{13′} are independently selected from hydrogen and unsubstituted C₁₋₆ alkyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₁₄, R_{14′} and R_{14″} are independently selected from hydrogen, unsubstituted C₁₋₆ alkyl, unsubstituted C₂₋₆ alkenyl, unsubstituted C₂₋₆ alkynyl, unsubstituted aryl, unsubstituted alkylaryl, unsubstituted cycloalkyl and unsubstituted alkylcycloalkyl, unsubstituted heterocyclyl and unsubstituted alkyheterocyclyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₁₄, R_{14′} and R_{14″} are independently selected from hydrogen, unsubstituted C₁₋₆ alkyl, unsubstituted aryl, unsubstituted alkylaryl, unsubstituted cycloalkyl and unsubstituted alkylcycloalkyl, unsubstituted heterocyclyl and unsubstituted alkyheterocyclyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

X is selected from a bond, substituted or unsubstituted aryl or —C(RₓR_{x′})—;
Rₓ is selected from substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
R_{x′} is selected from hydrogen, substituted or unsubstituted C₁₋₆ alkyl, substituted or unsubstituted C₂₋₆ alkenyl and substituted or unsubstituted C₂₋₆ alkynyl;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

m is 0, 1, 2, 3 or 4; preferably m is 0, 1 or 2;
and/or
n is 0, 1, 2, 3 or 4; preferably n is 0, 1, 2 or 3;
and/or
p is 0, 1, 2, 3 or 4; preferably p is 0;
and/or
W is nitrogen or —C(R_{4′})—; preferably W is nitrogen;
and/or
X is selected from a bond, substituted or unsubstituted aryl or —C(RₓR_{x′}); preferably X is selected from a bond, —CH(phenyl)-, —CH(benzyl)-, —CH(pyridine)-, —CH(thiophen)- and —CH(thiazole)-;
and/or
Rₓ is selected from substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl; preferably Rₓ is selected from substituted or unsubstituted aryl, substituted or unsubstituted alkylaryl and substituted or unsubstituted heterocyclyl; more preferably Rₓ is selected from substituted or unsubstituted phenyl, substituted or unsubstituted benzyl, substituted or unsubstituted pyridine, substituted or unsubstituted thiophen and substituted or unsubstituted thiazole;
and/or
R_{x′} is selected from hydrogen, substituted or unsubstituted C₁₋₆ alkyl, substituted or unsubstituted C₂₋₆ alkenyl and substituted or unsubstituted C₂₋₆ alkynyl; preferably R_{x′} is hydrogen;
   and/or
   Rₓₐ is independently selected from hydrogen, halogen, —OR₁₀, —CN, haloalkoxy, and —C(O)NR₁₀R_{10′}, preferably Rₓₐ is independently selected from hydrogen, fluorine, —O— methyl, —OCF₃, —C(O)NH₂ and —CN;
   and/or
   R₁ is selected from —NR₆R_{6′} and substituted or unsubstituted N-containing-heterocyclyl; preferably R₁ is selected from substituted or unsubstituted piperidin, substituted or unsubstituted tetrahydroisoquinoline, substituted or unsubstituted morpholine, —NH-methyl, —N(methyl)₂, —NH-ethyl, —NH₂, —N(methyl)(benzyl)-, —NHCH₂CH₂F and —NHCH₂CHF₂;
   and/or
   R₂ is selected from hydrogen, —NR₇R_{7′}, —CN, —CHR₇R_{7′} and substituted or unsubstituted heterocyclyl; preferably R₂ is selected from —NH₂, substituted or unsubstituted —NH-methyl, substituted or unsubstituted —N(methyl)₂, substituted or unsubstituted —N(ethyl)(methyl), substituted or unsubstituted —N(methyl)(propyl), substituted or unsubstituted azetidine; more preferably R₂ is selected from —NH— methyl, —N(methyl)₂, —N(methyl)(CH₂CH₂—OH), —N(methyl)(CH₂CH₂CH₂—OH) and substituted or unsubstituted azetidine;
   and/or
   R₃ is selected from hydrogen, substituted or unsubstituted C₁₋₆ alkyl, substituted or unsubstituted C₂₋₆ alkenyl and substituted or unsubstituted C₂₋₆ alkynyl; preferably R₃ is selected from hydrogen and substituted or unsubstituted C₁₋₆ alkyl; more preferably R₃ is substituted or unsubstituted methyl;
   and/or
   R₄ is selected from hydrogen, substituted or unsubstituted C₁₋₆ alkyl, substituted or unsubstituted C₂₋₆ alkenyl and substituted or unsubstituted C₂₋₆ alkynyl; preferably R₄ is selected from hydrogen and substituted or unsubstituted C₁₋₆ alkyl; more preferably R₄ is substituted or unsubstituted methyl;
   and/or
   R_{4′} is selected from hydrogen, substituted or unsubstituted C₁₋₆ alkyl, substituted or unsubstituted C₂₋₆ alkenyl and substituted or unsubstituted C₂₋₆ alkynyl;
   and/or
   R₅ and R_{5′} are independently selected from hydrogen, halogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl, substituted or unsubstituted alkyheterocyclyl, —OR₈, —NO₂, —NR₈R_{8′}, —NR₈C(O)R_{8′}, —NR₈S(O)₂R_{8′}, —S(O)₂NR₈R_{8′}, —NR₈C(O)NR_{8′}R_{8″}, —SR₈, —S(O)R₈, S(O)₂R₈, —CN, haloalkyl, haloalkoxy, —C(O)OR₈, —C(O)NR₈R_{8′}, —OCH₂CH₂OR₈, —NR₈S(O)₂NR_{8′}R_{8″} and C(CH₃)₂OR₈; preferably R₅ and R_{5′} are both hydrogen;
   and/or
   R₆ and R_{6′} are independently selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl; preferably R₆ and R_{6′} are independently selected from hydrogen, substituted or unsubstituted C₁₋₆ alkyl and substituted or unsubstituted alkylaryl; more preferably R₆ and R_{6′} are independently selected from hydrogen, substituted or unsubstituted methyl, substituted or unsubstituted ethyl and substituted or unsubstituted benzyl; even more preferably R₆ and R_{6′} are independently selected from hydrogen, substituted or unsubstituted methyl, substituted or unsubstituted ethyl, —CH₂CH₂F, —CH₂CHF₂ and substituted or unsubstituted benzyl
   and/or
   R₆ₐ is selected from substituted or unsubstituted cycloalkyl, substituted or unsubstituted alkylcycloalkyl, substituted or unsubstituted alkyheterocyclyl substituted or unsubstituted alkylaryl; preferably R₆ₐ is substituted or unsubstituted phenethyl;
   and/or
   R₇ and R_{7′} are independently selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl; preferably R₇ and R_{7′} are independently selected from hydrogen and substituted or unsubstituted C₁₋₆ alkyl; more preferably R₇ and R_{7′} are independently selected from hydrogen, substituted or unsubstituted methyl, substituted or unsubstituted ethyl, substituted or unsubstituted propyl; even more preferably R₇ and R_{7′} are independently selected from hydrogen, substituted or unsubstituted methyl, —CH₂CH₂OH and —CH₂CH₂CH₂OH;
   and/or
   R₈, R_{8′} and R_{8″} are independently selected from hydrogen, unsubstituted C₁₋₆ alkyl, unsubstituted C₂₋₆ alkenyl, and unsubstituted C₂₋₆ alkynyl;
   and/or
   R_{c} and R_{c′} are independently selected from hydrogen, substituted or unsubstituted C₁₋₆ alkyl, substituted or unsubstituted C₂₋₆ alkenyl and substituted or unsubstituted C₂₋₆ alkynyl; preferably, R_{c} and R_{c′} are both hydrogen;
   and/or
   R_{c} and R_{c′} may form with the carbon atom to which they are attached, a substituted or unsubstituted cycloalkyl; preferably, R_{c} and R_{c′} form a cyclopropyl;
   and/or
   R₉ and R_{9′} are independently selected from hydrogen, unsubstituted C₁₋₆ alkyl, unsubstituted C₂₋₆ alkenyl, and unsubstituted C₂₋₆ alkynyl; preferably R₉ and R_{9′} are independently selected from hydrogen and unsubstituted C₁₋₆ alkyl;
   and/or
   R₁₀, R_{10′} and R_{10″} are independently selected from hydrogen, unsubstituted C₁₋₆ alkyl, unsubstituted C₂₋₆ alkenyl, unsubstituted C₂₋₆ alkynyl, unsubstituted aryl, unsubstituted alkylaryl, unsubstituted cycloalkyl and unsubstituted alkylcycloalkyl, unsubstituted heterocyclyl and unsubstituted alkyheterocyclyl; preferably R₁₀, R_{10′} and R_{10″} are independently selected from hydrogen and unsubstituted C₁₋₆ alkyl; more preferably R₁₀ and R_{10″} are independently selected from hydrogen and unsubstituted methyl;
   and/or
   R₁₁, R_{11′} and R_{11″} are independently selected from hydrogen, unsubstituted C₁₋₆ alkyl, unsubstituted C₂₋₆ alkenyl and unsubstituted C₂₋₆ alkynyl; preferably R₁₁, R_{11′} and R_{11″} are independently selected from hydrogen and unsubstituted C₁₋₆ alkyl;
   and/or
   R₁₂, R_{12′}, and R_{12″} are independently selected from hydrogen, unsubstituted C₁₋₆ alkyl, and unsubstituted C₂₋₆ alkenyl and unsubstituted C₂₋₆ alkynyl; preferably R₁₂, R_{12′} and R₁₂, are independently selected from hydrogen and unsubstituted methyl;
   and/or
   R₁₃ and R_{13′} are independently selected from hydrogen, unsubstituted C₁₋₆ alkyl, unsubstituted C₂₋₆ alkenyl, and unsubstituted C₂₋₆ alkynyl;
   and/or
   R₁₄, R_{14′} and R_{14″} are independently selected from hydrogen, unsubstituted C₁₋₆ alkyl, unsubstituted C₂₋₆ alkenyl, unsubstituted C₂₋₆ alkynyl, unsubstituted aryl, unsubstituted alkylaryl, unsubstituted cycloalkyl and unsubstituted alkylcycloalkyl, unsubstituted heterocyclyl and unsubstituted alkyheterocyclyl;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

m is 0, 1, 2, 3 or 4;
and/or
n is 0, 1, 2, 3 or 4;
and/or
p is 0, 1, 2, 3 or 4;
and/or
W is nitrogen or —C(R_{4′})—;
and/or
X is selected from a bond, substituted or unsubstituted aryl or —C(RₓR_{x′});
wherein
the aryl is selected from phenyl, naphthyl, or anthracene; preferably is naphthyl and phenyl;
   and/or
   Rₓ is selected from substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl
   wherein
the alkyl is C₁₋₆ alkyl like methyl, ethyl, propyl, butyl, pentyl, hexyl, isopropyl, or 2-methylpropyl; more preferably the alkyl is methyl;
and/or
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or
the aryl is selected from phenyl, naphthyl, or anthracene; preferably is naphthyl and phenyl; preferably the aryl is phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline; preferably the heterocyclyl is pyridine, thiophen or thiazole;
   and/or
   R_{x′} is selected from hydrogen, substituted or unsubstituted C₁₋₆ alkyl, substituted or unsubstituted C₂₋₆ alkenyl and substituted or unsubstituted C₂₋₆ alkynyl;
   wherein
the C₁₋₆ alkyl is preferably selected from methyl, ethyl, propyl, butyl, pentyl, hexyl, isopropyl, or 2-methylpropyl,
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
   and/or
   R₁ is selected from —NR₆R_{6′} and substituted or unsubstituted N-containing-heterocyclyl;
   wherein
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline; even more preferably the heterocyclyl is piperidin, tetrahydroisoquinoline or morpholine;
and/or
   R₂ is selected from hydrogen, —NR₇R_{7′}, —CN, —CHR₇R_{7′} and substituted or unsubstituted heterocyclyl;
wherein
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline; even more preferably the heterocyclyl is azetidine;
and/or
   R₃ is selected from hydrogen, substituted or unsubstituted C₁₋₆ alkyl, substituted or unsubstituted C₂₋₆ alkenyl and substituted or unsubstituted C₂₋₆ alkynyl
   wherein
the C₁₋₆ alkyl is preferably selected from methyl, ethyl, propyl, butyl, pentyl, hexyl, isopropyl, or 2-methylpropyl, more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
   R₄ is selected from hydrogen, substituted or unsubstituted C₁₋₆ alkyl, substituted or unsubstituted C₂₋₆ alkenyl and substituted or unsubstituted C₂₋₆ alkynyl;
   wherein
the C₁₋₆ alkyl is preferably selected from methyl, ethyl, propyl, butyl, pentyl, hexyl, isopropyl, or 2-methylpropyl, more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
   R_{4′} is selected from hydrogen, substituted or unsubstituted C₁₋₆ alkyl, substituted or unsubstituted C₂₋₆ alkenyl and substituted or unsubstituted C₂₋₆ alkynyl;
   wherein
the C₁₋₆ alkyl is preferably selected from methyl, ethyl, propyl, butyl, pentyl, hexyl, isopropyl, or 2-methylpropyl; preferably methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
   R₅ and R_{5′} are independently selected from hydrogen, halogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl, substituted or unsubstituted alkyheterocyclyl, —OR₈, —NO₂, —NR₈R_{8′}, —NR₈C(O)R_{8′}, —NR₈S(O)₂R_{8′}, —S(O)₂NR₈R_{8′}, —NR₈C(O)NR_{8′}R_{8″}, —SR₈, —S(O)R₈, S(O)₂R₈, —CN, haloalkyl, haloalkoxy, —C(O)OR₈, —C(O)NR₈R_{8′}, —OCH₂CH₂OR₈, —NR₈S(O)₂NR_{8′}R_{8″} and C(CH₃)₂OR₈;
   wherein
the alkyl is C₁₋₆ alkyl like methyl, ethyl, propyl, butyl, pentyl, hexyl, isopropyl, or 2-methylpropyl;
and/or
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or
the aryl is selected from phenyl, naphthyl, or anthracene; preferably is naphthyl and phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole, oxopyrrolidine, pyrimidine, benzodioxolane, benzodioxane, carbazole and quinazoline;
and/or
   R_{c} and R_{c′} are independently selected from hydrogen, substituted or unsubstituted C₁₋₆ alkyl, substituted or unsubstituted C₂₋₆ alkenyl and substituted or unsubstituted C₂₋₆ alkynyl;
   wherein
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
   R_{c} and R_{c′} may form with the carbon atom to which they are attached, a substituted or unsubstituted cycloalkyl;
   wherein
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl; preferably the cycloalkyl is cyclopropyl;
and/or
   R₆ and R_{6′} are independently selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
   wherein
the alkyl is C₁₋₆ alkyl like methyl, ethyl, propyl, butyl, pentyl, hexyl, isopropyl, or 2-methylpropyl; preferably the alkyl is methyl;
and/or
the C₁₋₆ alkyl is preferably selected from methyl, ethyl, propyl, butyl, pentyl, hexyl, isopropyl, or 2-methylpropyl, more preferably the C₁₋₆ alkyl is methyl or ethyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or
the aryl is selected from phenyl, naphthyl, or anthracene; preferably is naphthyl and phenyl; more preferably the aryl is phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole, oxopyrrolidine, pyrimidine, benzodioxolane, benzodioxane, carbazole and quinazoline;
and/or
   R₆ₐ is selected from substituted or unsubstituted cycloalkyl, substituted or unsubstituted alkylcycloalkyl, substituted or unsubstituted alkyheterocyclyl substituted or unsubstituted alkylaryl;
   wherein
the alkyl is C₁₋₆ alkyl like methyl, ethyl, propyl, butyl, pentyl, hexyl, isopropyl, or 2-methylpropyl; preferably the alkyl is ethyl;
and/or
the C₁₋₆ alkyl is preferably selected from methyl, ethyl, propyl, butyl, pentyl, hexyl, isopropyl, or 2-methylpropyl; preferably the C₁₋₆ alkyl is methyl
and/or
the aryl is selected from phenyl, naphthyl, or anthracene; preferably is naphthyl and phenyl; preferably the aryl is phenyl;
and/or
   R₇ and R_{7′} are independently selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
   wherein
the alkyl is C₁₋₆ alkyl like methyl, ethyl, propyl, butyl, pentyl, hexyl, isopropyl, or 2-methylpropyl;
and/or
the C₁₋₆ alkyl is preferably selected from methyl, ethyl, propyl, butyl, pentyl, hexyl, isopropyl, or 2-methylpropyl, more preferably the C₁₋₆ alkyl is methyl, ethyl or propyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or
the aryl is selected from phenyl, naphthyl, or anthracene; preferably is naphthyl and phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole, oxopyrrolidine, pyrimidine, benzodioxolane, benzodioxane, carbazole and quinazoline;
and/or
   R₈, R_{8′} and R_{8″} are independently selected from hydrogen, unsubstituted C₁₋₆ alkyl, unsubstituted C₂₋₆ alkenyl and unsubstituted C₂₋₆ alkynyl;
   wherein
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
   R₉ and R_{9′} are independently selected from hydrogen, unsubstituted C₁₋₆ alkyl, unsubstituted C₂₋₆ alkenyl, and unsubstituted C₂₋₆ alkynyl;
   wherein
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
   and/or
   R₁₀, R_{10′} and R_{10″} are independently selected from hydrogen, unsubstituted C₁₋₆ alkyl, unsubstituted C₂₋₆ alkenyl, unsubstituted C₂₋₆ alkynyl, unsubstituted aryl, unsubstituted alkylaryl, unsubstituted cycloalkyl and unsubstituted alkylcycloalkyl, unsubstituted heterocyclyl and unsubstituted alkyheterocyclyl;
   wherein
the alkyl is C₁₋₆ alkyl like methyl, ethyl, propyl, butyl, pentyl, hexyl, isopropyl, or 2-methylpropyl;
and/or
the C₁₋₆ alkyl is preferably selected from methyl, ethyl, propyl, butyl, pentyl, hexyl, isopropyl, or 2-methylpropyl, more preferably the C₁₋₆ alkyl is methyl; and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or
the aryl is selected from phenyl, naphthyl, or anthracene; preferably is naphthyl and phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole, oxopyrrolidine, pyrimidine, benzodioxolane, benzodioxane, carbazole and quinazoline;
and/or
   R₁₁, R_{11′} and R_{11″} are independently selected from hydrogen, unsubstituted C₁₋₆ alkyl, unsubstituted C₂₋₆ alkenyl and unsubstituted C₂₋₆ alkynyl;
   wherein
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
   R₁₂, R_{12′} and R_{12″} are independently selected from hydrogen, unsubstituted C₁₋₆ alkyl, and unsubstituted C₂₋₆ alkenyl and unsubstituted C₂₋₆ alkynyl;
   wherein
the C₁₋₆ alkyl is preferably selected from methyl, ethyl, propyl, butyl, pentyl, hexyl, isopropyl, or 2-methylpropyl, more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
   R₁₃ and R_{13′} are independently selected from hydrogen, unsubstituted C₁₋₆ alkyl, unsubstituted C₂₋₆ alkenyl, and unsubstituted C₂₋₆ alkynyl;
wherein
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
   R₁₄, R_{14′} and R_{14″} are independently selected from hydrogen, unsubstituted C₁₋₆ alkyl, unsubstituted C₂₋₆ alkenyl, unsubstituted C₂₋₆ alkynyl, unsubstituted aryl, unsubstituted alkylaryl, unsubstituted cycloalkyl and unsubstituted alkylcycloalkyl, unsubstituted heterocyclyl and unsubstituted alkyheterocyclyl;
   wherein
the alkyl is C₁₋₆ alkyl like methyl, ethyl, propyl, butyl, pentyl, hexyl, isopropyl, or 2-methylpropyl;
and/or
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or
the aryl is selected from phenyl, naphthyl, or anthracene; preferably is naphthyl and phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole, oxopyrrolidine, pyrimidine, benzodioxolane, benzodioxane, carbazole and quinazoline;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in Rₓ as defined in any of the embodiments of the present invention,
the alkyl is C₁₋₆ alkyl like methyl, ethyl, propyl, butyl, pentyl, hexyl, isopropyl, or 2-methylpropyl; more preferably the alkyl is methyl;
and/or
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or
the aryl is selected from phenyl, naphthyl, or anthracene; preferably is naphthyl and phenyl; preferably the aryl is phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline; preferably the heterocyclyl is pyridine, thiophen or thiazole;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in Rₓ, as defined in any of the embodiments of the present invention,
the C₁₋₆ alkyl is preferably selected from methyl, ethyl, propyl, butyl, pentyl, hexyl, isopropyl, or 2-methylpropyl,
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₁ as defined in any of the embodiments of the present invention,
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline; even more preferably the heterocyclyl is piperidin, tetrahydroisoquinoline or morpholine;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₂ as defined in any of the embodiments of the present invention,
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline; even more preferably the heterocyclyl is azetidine;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₃ as defined in any of the embodiments of the present invention,
the C₁₋₆ alkyl is preferably selected from methyl, ethyl, propyl, butyl, pentyl, hexyl, isopropyl, or 2-methylpropyl, more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₄ as defined in any of the embodiments of the present invention,
the C₁₋₆ alkyl is preferably selected from methyl, ethyl, propyl, butyl, pentyl, hexyl, isopropyl, or 2-methylpropyl, more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R_{4′} as defined in any of the embodiments of the present invention,
the C₁₋₆ alkyl is preferably selected from methyl, ethyl, propyl, butyl, pentyl, hexyl, isopropyl, or 2-methylpropyl; preferably methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₅ and R_{5′} as defined in any of the embodiments of the present invention,
the alkyl is C₁₋₆ alkyl like methyl, ethyl, propyl, butyl, pentyl, hexyl, isopropyl, or 2-methylpropyl;
and/or
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or
the aryl is selected from phenyl, naphthyl, or anthracene; preferably is naphthyl and phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole, oxopyrrolidine, pyrimidine, benzodioxolane, benzodioxane, carbazole and quinazoline;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R_{c} and R_{c′} as defined in any of the embodiments of the present invention,
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R_{c} and R_{c′} as defined in any of the embodiments of the present invention,
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl; preferably the cycloalkyl is cyclopropyl;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₆ and R_{6′} as defined in any of the embodiments of the present invention,
the alkyl is C₁₋₆ alkyl like methyl, ethyl, propyl, butyl, pentyl, hexyl, isopropyl, or 2-methylpropyl; preferably the alkyl is methyl;
and/or
the C₁₋₆ alkyl is preferably selected from methyl, ethyl, propyl, butyl, pentyl, hexyl, isopropyl, or 2-methylpropyl, more preferably the C₁₋₆ alkyl is methyl or ethyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or
the aryl is selected from phenyl, naphthyl, or anthracene; preferably is naphthyl and phenyl; more preferably the aryl is phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole, oxopyrrolidine, pyrimidine, benzodioxolane, benzodioxane, carbazole and quinazoline;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₆ₐ as defined in any of the embodiments of the present invention,
the alkyl is C₁₋₆ alkyl like methyl, ethyl, propyl, butyl, pentyl, hexyl, isopropyl, or 2-methylpropyl; preferably the alkyl is ethyl;
and/or
the C₁₋₆ alkyl is preferably selected from methyl, ethyl, propyl, butyl, pentyl, hexyl, isopropyl, or 2-methylpropyl; preferably the C₁₋₆ alkyl is methyl
and/or
the aryl is selected from phenyl, naphthyl, or anthracene; preferably is naphthyl and phenyl; preferably the aryl is phenyl;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₇ and R_{7′} as defined in any of the embodiments of the present invention,
the alkyl is C₁₋₆ alkyl like methyl, ethyl, propyl, butyl, pentyl, hexyl, isopropyl, or 2-methylpropyl;
and/or
the C₁₋₆ alkyl is preferably selected from methyl, ethyl, propyl, butyl, pentyl, hexyl, isopropyl, or 2-methylpropyl, more preferably the C₁₋₆ alkyl is methyl, ethyl or propyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or
the aryl is selected from phenyl, naphthyl, or anthracene; preferably is naphthyl and phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole, oxopyrrolidine, pyrimidine, benzodioxolane, benzodioxane, carbazole and quinazoline;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₈, R_{8′} and R_{8″} as defined in any of the embodiments of the present invention,
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₉ and R_{9′}as defined in any of the embodiments of the present invention,
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₁₀, R_{10′} and R_{10″} as defined in any of the embodiments of the present invention,
the alkyl is C₁₋₆ alkyl like methyl, ethyl, propyl, butyl, pentyl, hexyl, isopropyl, or 2-methylpropyl;
and/or
the C₁₋₆ alkyl is preferably selected from methyl, ethyl, propyl, butyl, pentyl, hexyl, isopropyl, or 2-methylpropyl, more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or
the aryl is selected from phenyl, naphthyl, or anthracene; preferably is naphthyl and phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole, oxopyrrolidine, pyrimidine, benzodioxolane, benzodioxane, carbazole and quinazoline;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₁₁, R_{11′} and R_{11″} as defined in any of the embodiments of the present invention,
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₁₂, R_{12′} and R_{12″} as defined in any of the embodiments of the present invention,
the C₁₋₆ alkyl is preferably selected from methyl, ethyl, propyl, butyl, pentyl, hexyl, isopropyl, or 2-methylpropyl, more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₁₃ and R_{13′} as defined in any of the embodiments of the present invention,
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₁₄, R_{14′} and R_{14″} as defined in any of the embodiments of the present invention,
the alkyl is C₁₋₆ alkyl like methyl, ethyl, propyl, butyl, pentyl, hexyl, isopropyl, or 2-methylpropyl;
and/or
the C₁₋₆ alkyl is preferably selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or
the aryl is selected from phenyl, naphthyl, or anthracene; preferably is naphthyl and phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole, oxopyrrolidine, pyrimidine, benzodioxolane, benzodioxane, carbazole and quinazoline;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

m is 0, 1, 2, 3 or 4; preferably m is 0, 1 or 2;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

n is 0, 1, 2, 3 or 4; preferably n is 0, 1, 2 or 3;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

p is 0, 1, 2, 3 or 4; preferably p is 0;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

W is nitrogen or —C(R_{4′})—; preferably W is nitrogen;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

X is selected from a bond, substituted or unsubstituted aryl or —C(RₓR_{x′}); preferably X is selected from a bond, —CH(phenyl)-, —CH(benzyl)-, —CH(pyridine)-, —CH(thiophen)- and —CH(thiazole)-;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein
Rₓ is selected from substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl; preferably Rₓ is selected from substituted or unsubstituted aryl, substituted or unsubstituted alkylaryl and substituted or unsubstituted heterocyclyl; more preferably Rₓ is selected from substituted or unsubstituted phenyl, substituted or unsubstituted benzyl, substituted or unsubstituted pyridine, substituted or unsubstituted thiophen and substituted or unsubstituted thiazole;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein
R_{x′} is selected from hydrogen, substituted or unsubstituted C₁₋₆ alkyl, substituted or unsubstituted C₂₋₆ alkenyl and substituted or unsubstituted C₂₋₆ alkynyl; preferably R_{x′} is hydrogen;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

Rₓₐ is independently selected from hydrogen, halogen, —OR₁₀, —CN, haloalkoxy, and —C(O)NR₁₀R_{10′}, preferably Rₓₐ is independently selected from hydrogen, fluorine, —O— methyl, —OCF₃, —C(O)NH₂ and —CN;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₁ is selected from —NR₆R_{6′} and substituted or unsubstituted N-containing-heterocyclyl; preferably R₁ is selected from substituted or unsubstituted piperidin, substituted or unsubstituted tetrahydroisoquinoline, substituted or unsubstituted morpholine, —NH-methyl, —N(methyl)₂, —NH-ethyl, —NH₂, —N(methyl)(benzyl)-, —NHCH₂CH₂F and —NHCH₂CHF₂;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₂ is selected from hydrogen, —NR₇R_{7′}, —CN, —CHR₇R_{7′} and substituted or unsubstituted heterocyclyl; preferably R₂ is selected from —NH₂, substituted or unsubstituted —NH-methyl, substituted or unsubstituted —N(methyl)₂, substituted or unsubstituted —N(ethyl)(methyl), substituted or unsubstituted —N(methyl)(propyl), substituted or unsubstituted azetidine; more preferably R₂ is selected from —NH₂, —NH-methyl, —N(methyl)₂, —N(methyl)(CH₂CH₂—OH), —N(methyl)(CH₂CH₂CH₂—OH) and substituted or unsubstituted azetidine;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₃ is selected from hydrogen, substituted or unsubstituted C₁₋₆ alkyl, substituted or unsubstituted C₂₋₆ alkenyl and substituted or unsubstituted C₂₋₆ alkynyl; preferably R₃ is selected from hydrogen and substituted or unsubstituted C₁₋₆ alkyl; more preferably R₃ is substituted or unsubstituted methyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₄ is selected from hydrogen, substituted or unsubstituted C₁₋₆ alkyl, substituted or unsubstituted C₂₋₆ alkenyl and substituted or unsubstituted C₂₋₆ alkynyl; preferably R₄ is selected from hydrogen and substituted or unsubstituted C₁₋₆ alkyl; more preferably R₄ is substituted or unsubstituted methyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R_{4′} is selected from hydrogen, substituted or unsubstituted C₁₋₆ alkyl, substituted or unsubstituted C₂₋₆ alkenyl and substituted or unsubstituted C₂₋₆ alkynyl; preferably selected from hydrogen and substituted or unsubstituted methyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₅ and R_{5′} are independently selected from hydrogen, halogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl, substituted or unsubstituted alkyheterocyclyl, —OR₈, —NO₂, —NR₈R_{8′}, —NR₈C(O)R_{8′}, —NR₈S(O)₂R_{8′}, —S(O)₂NR₈R_{8′}, —NR₈C(O)NR_{8′}R_{8″}, —SR₈, —S(O)R₈, S(O)₂R₈, —CN, haloalkyl, haloalkoxy, —C(O)OR₈, —C(O)NR₈R_{8′}, —OCH₂CH₂OR₈, —NR₈S(O)₂NR_{8′}R_{8″}, and C(CH₃)₂OR₈; preferably R₅ and R_{5′} are both hydrogen;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₆ and R_{6′} are independently selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl; preferably R₆ and R_{6′} are independently selected from hydrogen, substituted or unsubstituted C₁₋₆ alkyl and substituted or unsubstituted alkylaryl; more preferably R₆ and R_{6′} are independently selected from hydrogen, substituted or unsubstituted methyl, substituted or unsubstituted ethyl and substituted or unsubstituted benzyl; even more preferably R₆ and R_{6′} are independently selected from hydrogen, substituted or unsubstituted methyl, substituted or unsubstituted ethyl, —CH₂CH₂F, —CH₂CHF₂ and substituted or unsubstituted benzyl
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₆ₐ is selected from substituted or unsubstituted cycloalkyl, substituted or unsubstituted alkylcycloalkyl, substituted or unsubstituted alkyheterocyclyl substituted or unsubstituted alkylaryl; preferably R₆ₐ is substituted or unsubstituted phenethyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₇ and R_{7′} are independently selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl; preferably R₇ and R_{7′} are independently selected from hydrogen and substituted or unsubstituted C₁₋₆ alkyl; more preferably R₇ and R_{7′} are independently selected from hydrogen, substituted or unsubstituted methyl, substituted or unsubstituted ethyl, substituted or unsubstituted propyl; even more preferably R₇ and R_{7′} are independently selected from hydrogen, substituted or unsubstituted methyl, —CH₂CH₂OH and —CH₂CH₂CH₂OH;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₈, R_{8′} and R_{8″} are independently selected from hydrogen, unsubstituted C₁₋₆ alkyl, unsubstituted C₂₋₆ alkenyl, and unsubstituted C₂₋₆ alkynyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R_{c} and R_{c′} are independently selected from hydrogen, substituted or unsubstituted C₁₋₆ alkyl, substituted or unsubstituted C₂₋₆ alkenyl and substituted or unsubstituted C₂₋₆ alkynyl; preferably, R_{c} and R_{c′} are both hydrogen;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R_{c} and R_{c′} may form with the carbon atom to which they are attached, a substituted or unsubstituted cycloalkyl; preferably, R_{c} and R_{c′} form a cyclopropyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₉ and R_{9′} are independently selected from hydrogen, unsubstituted C₁₋₆ alkyl, unsubstituted C₂₋₆ alkenyl, and unsubstituted C₂₋₆ alkynyl; preferably R₉ and R_{9′} are independently selected from hydrogen and unsubstituted C₁₋₆ alkyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₁₀, R_{10′} and R_{10″} are independently selected from hydrogen, unsubstituted C₁₋₆ alkyl, unsubstituted C₂₋₆ alkenyl, unsubstituted C₂₋₆ alkynyl, unsubstituted aryl, unsubstituted alkylaryl, unsubstituted cycloalkyl and unsubstituted alkylcycloalkyl, unsubstituted heterocyclyl and unsubstituted alkyheterocyclyl; preferably R₁₀, R_{10′} and R_{10″} are independently selected from hydrogen and unsubstituted C₁₋₆ alkyl; more preferably R₁₀ and R_{10′} are independently selected from hydrogen and unsubstituted methyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₁₁, R_{11′} and R_{11″} are independently selected from hydrogen, unsubstituted C₁₋₆ alkyl, unsubstituted C₂₋₆ alkenyl and unsubstituted C₂₋₆ alkynyl; preferably R₁₁, R_{11′} and R_{11″} are independently selected from hydrogen and unsubstituted C₁₋₆ alkyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₁₂, R_{12′} and R_{12″} are independently selected from hydrogen, unsubstituted C₁₋₆ alkyl, and unsubstituted C₂₋₆ alkenyl and unsubstituted C₂₋₆ alkynyl; preferably R₁₂, R_{12′} and R_{12″} are independently selected from hydrogen and unsubstituted methyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₁₃ and R_{13′} are independently selected from hydrogen, unsubstituted C₁₋₆ alkyl, unsubstituted C₂₋₆ alkenyl, and unsubstituted C₂₋₆ alkynyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₁₄, R_{14′} and R_{14″} are independently selected from hydrogen, unsubstituted C₁₋₆ alkyl, unsubstituted C₂₋₆ alkenyl, unsubstituted C₂₋₆ alkynyl, unsubstituted aryl, unsubstituted alkylaryl, unsubstituted cycloalkyl and unsubstituted alkylcycloalkyl, unsubstituted heterocyclyl and unsubstituted alkyheterocyclyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

m is 0, 1 or 2;
and
n is 0, 1, 2 or 3;
and
p is 0;
and
W is nitrogen;
and
X is selected from a bond, —CH(phenyl)-, —CH(benzyl)-, —CH(pyridine)-, —CH(thiophen)- and —CH(thiazole)-;
and
Rₓ is selected from substituted or unsubstituted aryl, substituted or unsubstituted alkylaryl and substituted or unsubstituted heterocyclyl; more preferably Rₓ is selected from substituted or unsubstituted phenyl, substituted or unsubstituted benzyl, substituted or unsubstituted pyridine, substituted or unsubstituted thiophen and substituted or unsubstituted thiazole;
and
R_{x′} is hydrogen;
and
Rₓₐ independently represents hydrogen, fluorine, —O-methyl, —OCF₃, —C(O)NH₂ or —CN;
and
R₁ is selected from substituted or unsubstituted piperidin, substituted or unsubstituted tetrahydroisoquinoline, substituted or unsubstituted morpholine, —NH-methyl, —N(methyl)₂, —NH-ethyl, —NH₂, —N(methyl)(benzyl)-, —NHCH₂CH₂F and —NHCH₂CHF₂;
and
R₂ is selected from —NH₂, substituted or unsubstituted —NH-methyl, substituted or unsubstituted —N(methyl)₂, substituted or unsubstituted —N(ethyl)(methyl), substituted or unsubstituted —N(methyl)(propyl), substituted or unsubstituted azetidine; more preferably R₂ is selected from —NH₂, —NH-methyl, —N(methyl)₂, —N(methyl)(CH₂CH₂—OH), —N(methyl)(CH₂CH₂CH₂—OH) and substituted or unsubstituted azetidine;
and
R₃ is selected from hydrogen and substituted or unsubstituted C₁₋₆ alkyl; more preferably R₃ is substituted or unsubstituted methyl;
and
R₄ is selected from hydrogen and substituted or unsubstituted C₁₋₆ alkyl; more preferably R₄ is substituted or unsubstituted methyl;
and
R₅ and R_{5′} are both hydrogen;
and
R₆ and R_{6′} are independently selected from hydrogen, substituted or unsubstituted C₁₋₆ alkyl and substituted or unsubstituted alkylaryl; more preferably R₆ and R_{6′} are independently selected from hydrogen, substituted or unsubstituted methyl, substituted or unsubstituted ethyl and substituted or unsubstituted benzyl; even more preferably R₆ and R_{6′} are independently selected from hydrogen, substituted or unsubstituted methyl, substituted or unsubstituted ethyl, —CH₂CH₂F, —CH₂CHF₂ and substituted or unsubstituted benzyl
and
R₆ₐ is substituted or unsubstituted phenethyl;
and
R₇ and R_{7′} are independently selected from hydrogen and substituted or unsubstituted C₁₋₆ alkyl; more preferably R₇ and R_{7′} are independently selected from hydrogen, substituted or unsubstituted methyl, substituted or unsubstituted ethyl, substituted or unsubstituted propyl; even more preferably R₇ and R_{7′} are independently selected from hydrogen, substituted or unsubstituted methyl, —CH₂CH₂OH and —CH₂CH₂CH₂OH;
and
R_{c} and R_{c′} are both hydrogen or R_{c} and R_{c′} form with the carbon atom to which they are attached, a substituted or unsubstituted cycloalkyl; preferably a cyclopropyl;
and
R₁₀, R_{10′} and R_{10″} are independently selected from hydrogen and unsubstituted C₁₋₆ alkyl; more preferably R₁₀ and R_{10′} are independently selected from hydrogen and unsubstituted methyl;
and
R₁₂, R_{12′} and R_{12″} are independently selected from hydrogen and unsubstituted C₁₋₆ alkyl; preferably selected from hydrogen and unsubstituted methyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment

m is 0, 1 or 2;

In a preferred embodiment

n is 0, 1, 2 or 3;

In a preferred embodiment

p is 0 or 1;

In a preferred embodiment

W is nitrogen or —CR4′, preferably nitrogen or substituted or unsubstituted methyl, more preferably nitrogen or unsubstituted methyl;

In a preferred embodiment

X is selected from a bond, —CH(phenyl)-, —CH(benzyl)-, —CH(pyridine)-, —CH(thiophen)- and —CH(thiazole)-;

In a preferred embodiment

Rₓ is selected from substituted or unsubstituted aryl, substituted or unsubstituted alkylaryl and substituted or unsubstituted heterocyclyl; more preferably Rₓ is selected from substituted or unsubstituted phenyl, substituted or unsubstituted benzyl, substituted or unsubstituted pyridine, substituted or unsubstituted thiophen and substituted or unsubstituted thiazole;

In a preferred embodiment

R_{x′} is hydrogen;

In a preferred embodiment

Rₓ is selected from substituted or unsubstituted aryl, substituted or unsubstituted alkylaryl and substituted or unsubstituted heterocyclyl; more preferably Rₓ is selected from substituted or unsubstituted phenyl, substituted or unsubstituted benzyl, substituted or unsubstituted pyridine, substituted or unsubstituted thiophen and substituted or unsubstituted thiazole;

In a preferred embodiment

Rₓ is a group selected from:

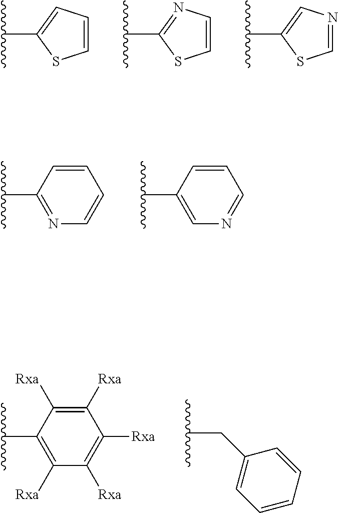

In a preferred embodiment

R_{x′} is hydrogen;

In a preferred embodiment

Rₓₐ is independently selected from hydrogen, halogen, —OR₁₀, —CN, haloalkoxy and —C(O)NR₁₀R_{10′}; preferably Rₓₐ is independently selected from hydrogen, fluorine, —O— methyl, —CN, —C(O)NH₂ and —O—CF₃.

In a preferred embodiment

R₁ is selected from substituted or unsubstituted piperidin, substituted or unsubstituted tetrahydroisoquinoline, substituted or unsubstituted morpholine, —NH-methyl, —N(methyl)₂, —NH-ethyl, —NH₂, —N(methyl)(benzyl)-, —NHCH₂CH₂F and —NHCH₂CHF₂;

In a preferred embodiment

R₁ is selected from —NH-methyl, —N(methyl)₂, —NH-ethyl, —NH₂, —N(methyl)(benzyl)-, —NHCH₂CH₂F and —NHCH₂CHF₂ or a substituted or unsubstituted group selected from

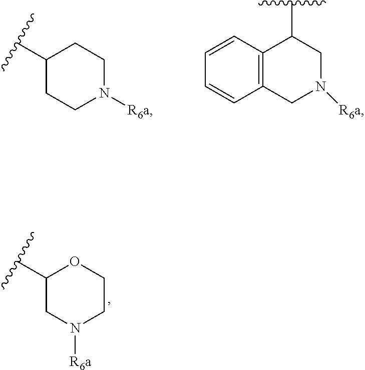

more preferably

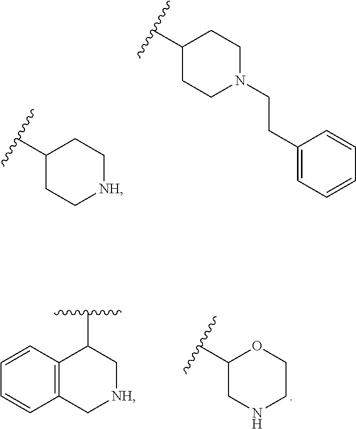 [SMILES: *C1CCNCC1 ; *C1CCN(CCc2ccccc2)CC1 ; *C1CNCc2ccccc21 ; *C1CNCCO1]

In a preferred embodiment

R₂ is selected from hydrogen, substituted or unsubstituted methyl, —NH₂, substituted or unsubstituted —NH-methyl, substituted or unsubstituted —N(methyl)₂, substituted or unsubstituted —N(ethyl)(methyl), substituted or unsubstituted —N(methyl)(propyl), substituted or unsubstituted azetidine; more preferably R₂ is selected from hydrogen, unsubstituted methyl, —NH-methyl, —N(methyl)₂, —N(methyl)(CH₂CH₂—OH), —N(methyl)(CH₂CH₂CH₂—OH) and substituted or unsubstituted azetidine;

In a preferred embodiment

R₂ is selected from hydrogen, substituted or unsubstituted methyl, —NH₂, substituted or unsubstituted —NH-methyl, substituted or unsubstituted —N(methyl)₂, substituted or unsubstituted —N(ethyl)(methyl), substituted or unsubstituted —N(methyl)(propyl), substituted or unsubstituted azetidine; more preferably R₂ is selected from hydrogen, unsubstituted methyl, —NH-methyl, —N(methyl)₂, —N(methyl)(CH₂CH₂—OH), N(methyl)(CH₂CH₂CH₂—OH) and substituted or unsubstituted azetidine of formula

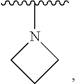 [SMILES: *N1CCC1]

preferably

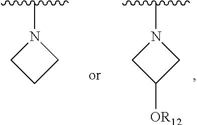

more preferably

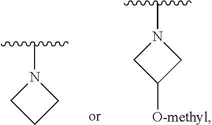

even more preferably

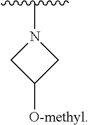

In a preferred embodiment
R₃ is selected from hydrogen and substituted or unsubstituted C₁₋₆ alkyl; preferably R₃ is substituted or unsubstituted methyl, more preferably unsubstituted methyl.

In a preferred embodiment

R₄ is selected from hydrogen and substituted or unsubstituted C₁₋₆ alkyl; preferably R₄ is substituted or unsubstituted methyl, more preferably unsubstituted methyl.

In a preferred embodiment

R_{4′} is selected from hydrogen and substituted or unsubstituted C₁₋₆ alkyl; preferably R_{4′} is substituted or unsubstituted methyl, more preferably unsubstituted methyl.

In a preferred embodiment

R_{4′} is selected from hydrogen and substituted or unsubstituted C₁₋₆ alkyl; preferably R_{4′} is hydrogen or substituted or unsubstituted methyl, more preferably hydrogen or unsubstituted methyl.

In a preferred embodiment

R₃ is selected from hydrogen and substituted or unsubstituted C₁₋₆ alkyl; preferably R₃ is substituted or unsubstituted methyl, more preferably unsubstituted methyl, while R₄ is selected from hydrogen and substituted or unsubstituted C₁₋₆ alkyl; preferably R₄ is substituted or unsubstituted methyl, more preferably unsubstituted methyl.

In a preferred embodiment

R₃ and R₄ are both unsubstituted methyl.

In a preferred embodiment

R₅ and R_{5′} are both hydrogen;

In a preferred embodiment

R₆ and R_{6′} are independently selected from hydrogen, substituted or unsubstituted C₁₋₆ alkyl and substituted or unsubstituted alkylaryl; more preferably R₆ and R_{6′} are independently selected from hydrogen, substituted or unsubstituted methyl, substituted or unsubstituted ethyl and substituted or unsubstituted benzyl; even more preferably R₆ and R_{6′} are independently selected from hydrogen, substituted or unsubstituted methyl, substituted or unsubstituted ethyl, —CH₂CH₂F, —CH₂CHF₂ and substituted or unsubstituted benzyl.

In a preferred embodiment

R₆ is selected from hydrogen, substituted or unsubstituted C₁₋₆ alkyl and substituted or unsubstituted alkylaryl; more preferably R₆ is selected from hydrogen, substituted or unsubstituted methyl, substituted or unsubstituted ethyl and substituted or unsubstituted benzyl; even more preferably R₆ is selected from hydrogen, substituted or unsubstituted methyl, substituted or unsubstituted ethyl, —CH₂CH₂F, —CH₂CHF₂ and substituted or unsubstituted benzyl.

In a preferred embodiment

R_{6′} is selected from hydrogen and substituted or unsubstituted C₁₋₆ alkyl; more preferably R_{6′} is selected from hydrogen and substituted or unsubstituted methyl; even more preferably R_{6′} is selected from hydrogen and unsubstituted methyl.

In a preferred embodiment

R₆ is selected from hydrogen, substituted or unsubstituted C₁₋₆ alkyl and substituted or unsubstituted alkylaryl; more preferably R₆ is selected from hydrogen, substituted or unsubstituted methyl, substituted or unsubstituted ethyl and substituted or unsubstituted benzyl; even more preferably R₆ is selected from hydrogen, substituted or unsubstituted methyl, substituted or unsubstituted ethyl, —CH₂CH₂F, —CH₂CHF₂ and substituted or unsubstituted benzyl, while R_{6′} is selected from hydrogen and substituted or unsubstituted C₁₋₆ alkyl; more preferably from hydrogen and substituted or unsubstituted methyl; even more preferably from hydrogen and unsubstituted methyl.

In a preferred embodiment

R₆ is selected from hydrogen, substituted or unsubstituted C₁₋₆ alkyl and substituted or unsubstituted alkylaryl; more preferably R₆ is selected from hydrogen, substituted or unsubstituted methyl, substituted or unsubstituted ethyl and substituted or unsubstituted benzyl; even more preferably R₆ is selected from hydrogen, substituted or unsubstituted methyl, substituted or unsubstituted ethyl, —CH₂CH₂F, —CH₂CHF₂ and substituted or unsubstituted benzyl, while R_{6′} is hydrogen.

In a preferred embodiment

R₆ is selected from hydrogen, substituted or unsubstituted C₁₋₆ alkyl and substituted or unsubstituted alkylaryl; more preferably R₆ is selected from hydrogen, substituted or unsubstituted methyl, substituted or unsubstituted ethyl and substituted or unsubstituted benzyl; even more preferably R₆ is selected from hydrogen, substituted or unsubstituted methyl, substituted or unsubstituted ethyl, —CH₂CH₂F, —CH₂CHF₂ and substituted or unsubstituted benzyl, while R_{6′} is substituted or unsubstituted C₁₋₆ alkyl; more preferably substituted or unsubstituted methyl; even more preferably unsubstituted methyl.

In a preferred embodiment

R₆ is substituted or unsubstituted C₁₋₆ alkyl; more preferably substituted or unsubstituted methyl, while R_{6′} is hydrogen.

In a preferred embodiment

R₆ and R_{6′} are both hydrogen.

In a preferred embodiment

R₆ is a substituted or unsubstituted

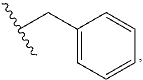 [SMILES: *Cc1ccccc1]

more preferably unsubstituted

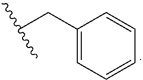 [SMILES: *Cc1ccccc1]

In a preferred embodiment

R₆ₐ is substituted or unsubstituted alkylaryl; preferably substituted or unsubstituted phenethyl; more preferably unsubstituted phenethyl of formula

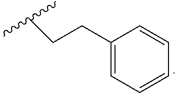 [SMILES: *CCc1ccccc1]

In a preferred embodiment

R₇ and R_{7′} are independently selected from hydrogen and substituted or unsubstituted C₁₋₆ alkyl; more preferably R₇ and R_{7′} are independently selected from hydrogen, substituted or unsubstituted methyl, substituted or unsubstituted ethyl, substituted or unsubstituted propyl; even more preferably R₇ and R_{7′} are independently selected from hydrogen, substituted or unsubstituted methyl, —CH₂CH₂OH and —CH₂CH₂CH₂OH;

In a preferred embodiment

R₇ is selected from hydrogen and substituted or unsubstituted C₁₋₆ alkyl; more preferably R₇ is selected from hydrogen, substituted or unsubstituted methyl, substituted or unsubstituted ethyl, substituted or unsubstituted propyl; even more preferably R₇ is selected from hydrogen, substituted or unsubstituted methyl, —CH₂CH₂OH and —CH₂CH₂CH₂OH;

In a preferred embodiment

R_{7′} is selected from hydrogen and substituted or unsubstituted C₁₋₆ alkyl; more preferably R_{7′} is selected from hydrogen and substituted or unsubstituted methyl; even more preferably R_{7′} is selected from hydrogen and substituted or unsubstituted methyl;

In a preferred embodiment

R₇ is selected from hydrogen and substituted or unsubstituted C₁₋₆ alkyl; more preferably R₇ is selected from hydrogen, substituted or unsubstituted methyl, substituted or unsubstituted ethyl, substituted or unsubstituted propyl; even more preferably R₇ is selected from hydrogen, substituted or unsubstituted methyl, —CH₂CH₂OH and —CH₂CH₂CH₂OH, while R_{7′} is selected from hydrogen and substituted or unsubstituted C₁₋₆ alkyl; more preferably R_{7′} is selected from hydrogen and substituted or unsubstituted methyl; even more preferably R_{7′} is selected from hydrogen and substituted or unsubstituted methyl;

In a preferred embodiment

R₇ is selected from hydrogen and substituted or unsubstituted C₁₋₆ alkyl; more preferably R₇ is selected from hydrogen, substituted or unsubstituted methyl, substituted or unsubstituted ethyl, substituted or unsubstituted propyl; even more preferably R₇ is selected from hydrogen, substituted or unsubstituted methyl, —CH₂CH₂OH and —CH₂CH₂CH₂OH, while R_{7′} is substituted or unsubstituted C₁₋₆ alkyl; preferably substituted or unsubstituted methyl; even more preferably substituted or unsubstituted methyl;

In a preferred embodiment

R₇ and R_{7′} are both substituted or unsubstituted C₁₋₆ alkyl; preferably substituted or unsubstituted methyl; more preferably unsubstituted methyl;

In a preferred embodiment

R_{c} is hydrogen.

In a preferred embodiment

R_{c′} is hydrogen.

In a preferred embodiment

R_{c} and R_{c′} are both hydrogen.

In a preferred embodiment

R_{c} and R_{c′} form a cyclopropyl.

In a preferred embodiment

R₁₀ is selected from hydrogen and unsubstituted C₁₋₆ alkyl; preferably selected from hydrogen and unsubstituted methyl.

In a preferred embodiment

R_{10′} is hydrogen.

In a preferred embodiment

R₁₀ is selected from hydrogen and unsubstituted C₁₋₆ alkyl; preferably selected from hydrogen and unsubstituted methyl, while R_{10′} is hydrogen.

In a preferred embodiment

R₁₀ and R_{10′} are both hydrogen.

In a preferred embodiment

R₁₂ is selected from hydrogen and unsubstituted methyl;

In an particular embodiment

the halogen is fluorine, chlorine, iodine or bromine.

In an particular embodiment

the halogen is fluorine.

In an particular embodiment

the haloalcoxy is —OCF₃.

In a preferred further embodiment, the compounds of the general Formula (I) are selected from

| EX | Structure | Chemical name |
|---|---|---|
| 1 | 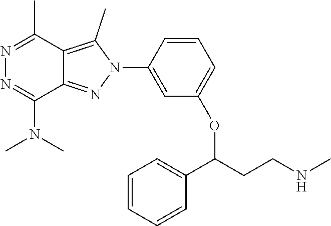 [SMILES: CNCCC(Oc1cccc(-n2nc3c(N(C)C)nnc(C)c3c2C)c1)c1ccccc1] | N,N,3,4-Tetramethyl-2-(3-(3- (methylamino)-1- phenylpropoxy)phenyl)-2H- pyrazolo[3,4-d]pyridazin-7-amine |
| 2 | 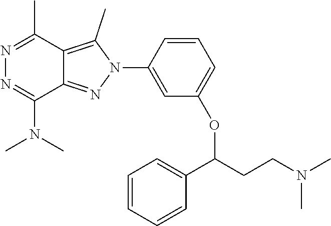 [SMILES: Cc1nnc(N(C)C)c2nn(-c3cccc(OC(CCN(C)C)c4ccccc4)c3)c(C)c12] | 2-(3-(3-(dimethylamino)-1- phenylpropoxy)phenyl)-N,N,3,4- tetramethyl-2H-pyrazolo[3,4- d]pyridazin-7-amine |
| 3 | 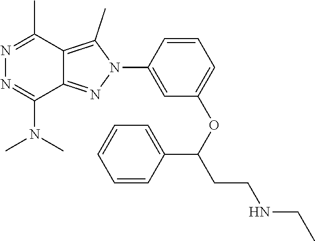 [SMILES: CCNCCC(Oc1cccc(-n2nc3c(N(C)C)nnc(C)c3c2C)c1)c1ccccc1] | 2-(3-(3-(ethylamino)-1- phenylpropoxy)phenyl)-N,N,3,4- tetramethyl-2H-pyrazolo[3,4- d]pyridazin-7-amine |
| 4 | 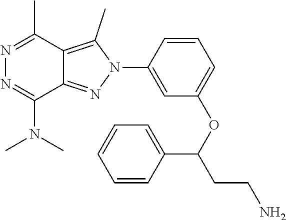 [SMILES: Cc1nnc(N(C)C)c2nn(-c3cccc(OC(CCN)c4ccccc4)c3)c(C)c12] | 2-(3-(3-amino-1- phenylpropoxy)phenyl)-N,N,3,4- tetramethyl-2H-pyrazolo[3,4- d]pyridazin-7-amine |
| 5 | 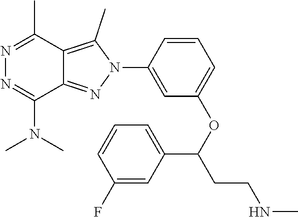 [SMILES: CNCCC(Oc1cccc(-n2nc3c(N(C)C)nnc(C)c3c2C)c1)c1cccc(F)c1] | 2-(3-(1-(3-fluorophenyl)-3- (methylamino)propoxy)phenyl)- N,N,3,4-tetramethyl-2H-pyrazolo[3,4- d]pyridazin-7-amine |
| 6 | 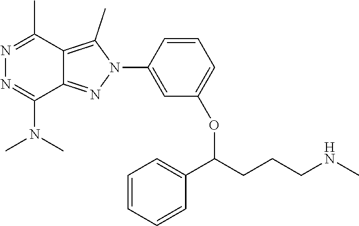 [SMILES: CNCCCC(Oc1cccc(-n2nc3c(N(C)C)nnc(C)c3c2C)c1)c1ccccc1] | N,N,3,4-tetramethyl-2-(3-(4- (methylamino)-1-phenylbutoxy)phenyl)- 2H-pyrazolo[3,4-d]pyridazin-7-amine |
| 7 | 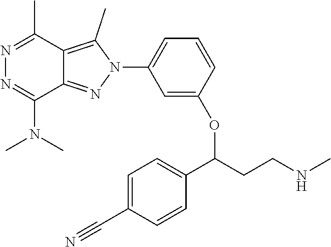 [SMILES: CNCCC(Oc1cccc(-n2nc3c(N(C)C)nnc(C)c3c2C)c1)c1ccc(C#N)cc1] | 4-(1-(3-(7-(dimethylamino)-3,4- dimethyl-2H-pyrazolo[3,4-d]pyridazin- 2-yl)phenoxy)-3- (methylamino)propyl)benzonitrile |
| 8 | 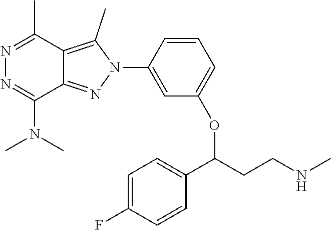 [SMILES: CNCCC(Oc1cccc(-n2nc3c(N(C)C)nnc(C)c3c2C)c1)c1ccc(F)cc1] | 2-(3-(1-(4-fluorophenyl)-3- (methylamino)propoxy)phenyl)- N,N,3,4-tetramethyl-2H-pyrazolo[3,4- d]pyridazin-7-amine |
| 9 | 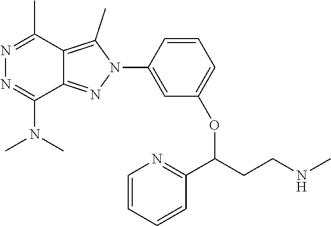 [SMILES: CNCCC(Oc1cccc(-n2nc3c(N(C)C)nnc(C)c3c2C)c1)c1ccccn1] | N,N,3,4-tetramethyl-2-(3-(3- (methylamino)-1-(pyridin-2- yl)propoxy)phenyl)-2H-pyrazolo[3,4- d]pyridazin-7-amine |
| 10 | 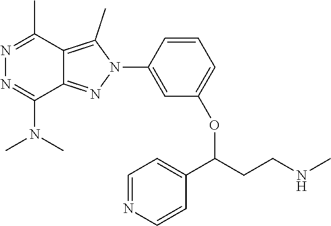 [SMILES: CNCCC(Oc1cccc(-n2nc3c(N(C)C)nnc(C)c3c2C)c1)c1ccncc1] | N,N,3,4-tetramethyl-2-(3-(3- (methylamino)-1-(pyridin-4- yl)propoxy)phenyl)-2H-pyrazolo[3,4- d]pyridazin-7-amine |
| 11 | 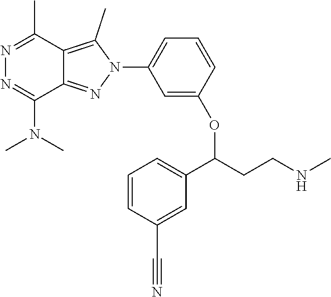 [SMILES: CNCCC(Oc1cccc(-n2nc3c(N(C)C)nnc(C)c3c2C)c1)c1cccc(C#N)c1] | 3-(1-(3-(7-(dimethylamino)-3,4- dimethyl-2H-pyrazolo[3,4-d]pyridazin- 2-yl)phenoxy)-3- (methylamino)propyl)benzonitrile |
| 12 | 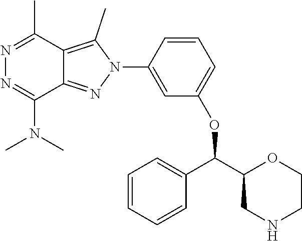 [SMILES: Cc1nnc(N(C)C)c2nn(-c3cccc(O[C@H](c4ccccc4)[C@@H]4CNCCO4)c3)c(C)c12] | N,N,3,4-tetramethyl-2-(3-((R)-((S)- morpholin-2- yl)(phenyl)methoxy)phenyl)-2H- pyrazolo[3,4-d]pyridazin-7-amine ⁽¹⁾ |
| 13 | 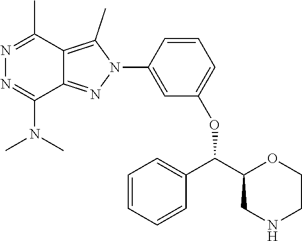 [SMILES: Cc1nnc(N(C)C)c2nn(-c3cccc(O[C@@H](c4ccccc4)[C@@H]4CNCCO4)c3)c(C)c12] | N,N,3,4-tetramethyl-2-(3-((S)-((S)- morpholin-2- yl)(phenyl)methoxy)phenyl)-2H- pyrazolo[3,4-d]pyridazin-7-amine ⁽²⁾ |
| 14 | 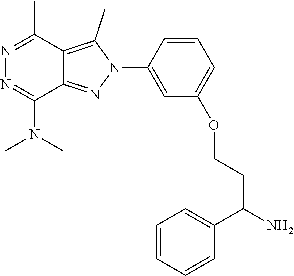 [SMILES: Cc1nnc(N(C)C)c2nn(-c3cccc(OCCC(N)c4ccccc4)c3)c(C)c12] | 2-(3-(3-amino-3- phenylpropoxy)phenyl)-N,N,3,4- tetramethyl-2H-pyrazolo[3,4- d]pyridazin-7-amine |
| 15 | 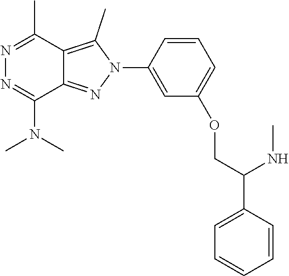 [SMILES: CNC(COc1cccc(-n2nc3c(N(C)C)nnc(C)c3c2C)c1)c1ccccc1] | N,N,3,4-tetramethyl-2-(3-(2- (methylamino)-2-phenylethoxy)phenyl)- 2H-pyrazolo[3,4-d]pyridazin-7-amine |
| 16 | 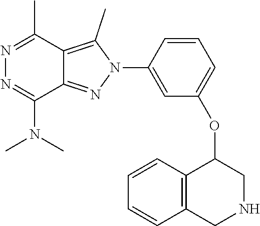 [SMILES: Cc1nnc(N(C)C)c2nn(-c3cccc(OC4CNCc5ccccc54)c3)c(C)c12] | N,N,3,4-tetramethyl-2-(3-((1,2,3,4- tetrahydroisoquinolin-4-yl)oxy)phenyl)- 2H-pyrazolo[3,4-d]pyridazin-7-amine |
| 17 | 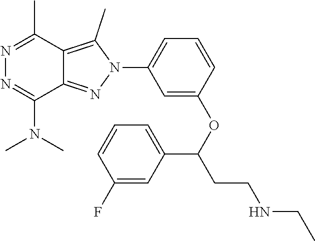 [SMILES: CCNCCC(Oc1cccc(-n2nc3c(N(C)C)nnc(C)c3c2C)c1)c1cccc(F)c1] | 2-(3-(3-(ethylamino)-1-(3- fluorophenyl)propoxy)phenyl)-N,N,3,4- tetramethyl-2H-pyrazolo[3,4- d]pyridazin-7-amine |
| 18 | 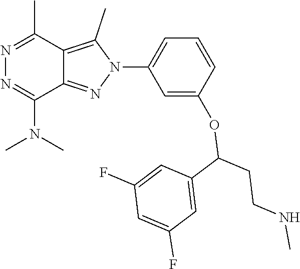 [SMILES: CNCCC(Oc1cccc(-n2nc3c(N(C)C)nnc(C)c3c2C)c1)c1cc(F)cc(F)c1] | 2-(3-(1-(3,5-difluorophenyl)-3- (methylamino)propoxy)phenyl)- N,N,3,4-tetramethyl-2H-pyrazolo[3,4- d]pyridazin-7-amine |
| 19 | 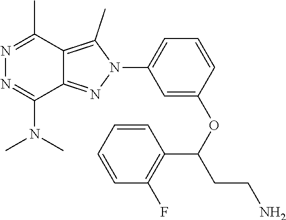 [SMILES: Cc1nnc(N(C)C)c2nn(-c3cccc(OC(CCN)c4ccccc4F)c3)c(C)c12] | 2-(3-(3-amino-1-(2- fluorophenyl)propoxy)phenyl)-N,N,3,4- tetramethyl-2H-pyrazolo[3,4- d]pyridazin-7-amine |
| 20 | 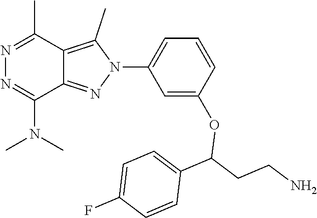 [SMILES: Cc1nnc(N(C)C)c2nn(-c3cccc(OC(CCN)c4ccc(F)cc4)c3)c(C)c12] | 2-(3-(3-amino-1-(4- fluorophenyl)propoxy)phenyl)-N,N,3,4- tetramethyl-2H-pyrazolo[3,4- d]pyridazin-7-amine |
| 21 | 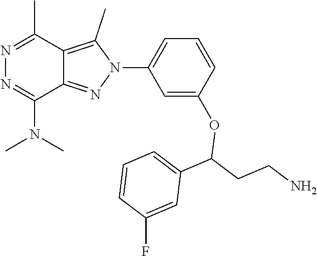 [SMILES: Cc1nnc(N(C)C)c2nn(-c3cccc(OC(CCN)c4cccc(F)c4)c3)c(C)c12] | 2-(3-(3-amino-1-(3- fluorophenyl)propoxy)phenyl)-N,N,3,4- tetramethyl-2H-pyrazolo[3,4- d]pyridazin-7-amine |
| 22 | 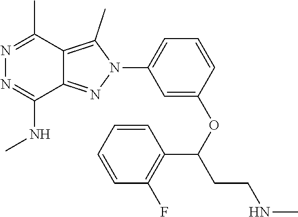 [SMILES: CNCCC(Oc1cccc(-n2nc3c(NC)nnc(C)c3c2C)c1)c1ccccc1F] | 2-(3-(1-(2-fluorophenyl)-3- (methylamino)propoxy)phenyl)-N,3,4- trimethyl-2H-pyrazolo[3,4-d]pyridazin- 7-amine |
| 23 | 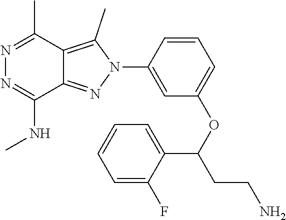 [SMILES: CNc1nnc(C)c2c(C)n(-c3cccc(OC(CCN)c4ccccc4F)c3)nc12] | 2-(3-(3-amino-1-(2- fluorophenyl)propoxy)phenyl)-N,3,4- trimethyl-2H-pyrazolo[3,4-d]pyridazin- 7-amine |
| 24 | 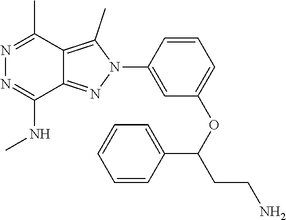 [SMILES: CNc1nnc(C)c2c(C)n(-c3cccc(OC(CCN)c4ccccc4)c3)nc12] | 2-(3-(3-amino-1- phenylpropoxy)phenyl)-N,3,4-trimethyl- 2H-pyrazolo[3,4-d]pyridazin-7-amine |
| 25 | 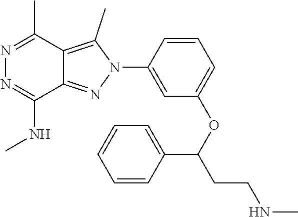 [SMILES: CNCCC(Oc1cccc(-n2nc3c(NC)nnc(C)c3c2C)c1)c1ccccc1] | N,3,4-trimethyl-2-(3-(3-(methylamino)- 1-phenylpropoxy)phenyl)-2H- pyrazolo[3,4-d]pyridazin-7-amine |
| 26 | 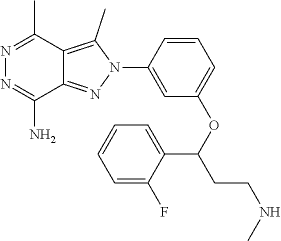 [SMILES: CNCCC(Oc1cccc(-n2nc3c(N)nnc(C)c3c2C)c1)c1ccccc1F] | 2-(3-(1-(2-fluorophenyl)-3- (methylamino)propoxy)phenyl)-3,4- dimethyl-2H-pyrazolo[3,4-d]pyridazin- 7-amine |
| 27 | 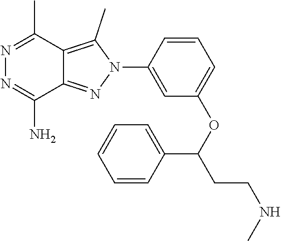 [SMILES: CNCCC(Oc1cccc(-n2nc3c(N)nnc(C)c3c2C)c1)c1ccccc1] | 3,4-dimethyl-2-(3-(3-(methylamino)-1- phenylpropoxy)phenyl)-2H- pyrazolo[3,4-d]pyridazin-7-amine |
| 28 | 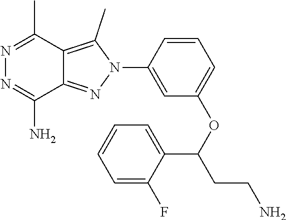 [SMILES: Cc1nnc(N)c2nn(-c3cccc(OC(CCN)c4ccccc4F)c3)c(C)c12] | 2-(3-(3-amino-1-(2- fluorophenyl)propoxy)phenyl)-3,4- dimethyl-2H-pyrazolo[3,4-d]pyridazin- 7-amine |
| 29 | 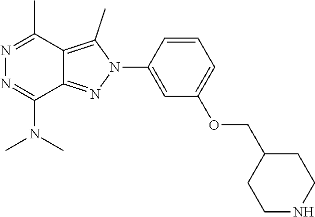 [SMILES: Cc1nnc(N(C)C)c2nn(-c3cccc(OCC4CCNCC4)c3)c(C)c12] | N,N,3,4-tetramethyl-2-(3-(piperidin-4- ylmethoxy)phenyl)-2H-pyrazolo[3,4- d]pyridazin-7-amine |
| 30 | 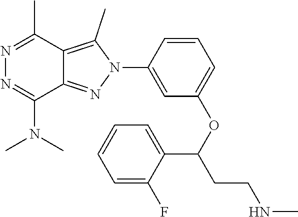 [SMILES: CNCCC(Oc1cccc(-n2nc3c(N(C)C)nnc(C)c3c2C)c1)c1ccccc1F] | 2-(3-(1-(2-Fluorophenyl)-3- (methylamino)propoxy)phenyl)- N,N,3,4-tetramethyl-2H-pyrazolo[3,4- d]pyridazin-7-amine |
| 31 | 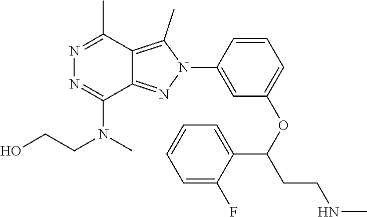 [SMILES: CNCCC(Oc1cccc(-n2nc3c(N(C)CCO)nnc(C)c3c2C)c1)c1ccccc1F] | 2-((2-(3-(1-(2-fluorophenyl)-3- (methylamino)propoxy)phenyl)-3,4- dimethyl-2H-pyrazolo[3,4-d]pyridazin- 7-yl)(methyl)amino)ethanol |
| 32 | 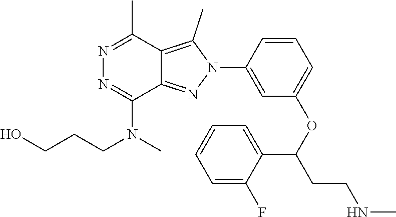 [SMILES: CNCCC(Oc1cccc(-n2nc3c(N(C)CCCO)nnc(C)c3c2C)c1)c1ccccc1F] | 3-((2-(3-(1-(2-fluorophenyl)-3- (methylamino)propoxy)phenyl)-3,4- dimethyl-2H-pyrazolo[3,4-d]pyridazin- 7-yl)(methyl)amino)propan-1-ol |
| 33 | 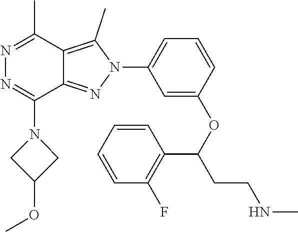 [SMILES: CNCCC(Oc1cccc(-n2nc3c(N4CC(OC)C4)nnc(C)c3c2C)c1)c1ccccc1F] | 3-(2-fluorophenyl)-3-(3-(7-(3- methoxyazetidin-1-yl)-3,4-dimethyl-2H- pyrazolo[3,4-d]pyridazin-2-yl)phenoxy)- N-methylpropan-1-amine |
| 34 | 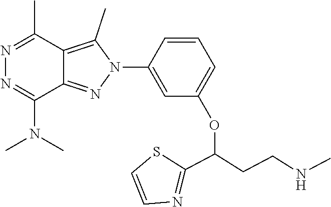 [SMILES: CNCCC(Oc1cccc(-n2nc3c(N(C)C)nnc(C)c3c2C)c1)c1nccs1] | N,N,3,4-Tetramethyl-2-(3-(3- (methylamino)-1-(thiazol-2- yl)propoxy)phenyl)-2H-pyrazolo[3,4- d]pyridazin-7-amine |
| 35 | 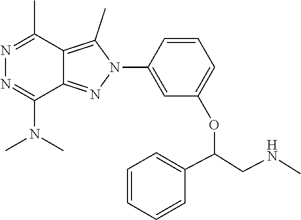 [SMILES: CNCC(Oc1cccc(-n2nc3c(N(C)C)nnc(C)c3c2C)c1)c1ccccc1] | N,N,3,4-tetramethyl-2-(3-(2- (methylamino)-1-phenylethoxy)phenyl)- 2H-pyrazolo[3,4-d]pyridazin-7-amine |
| 36 | 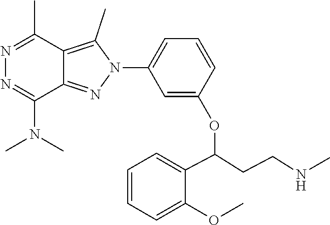 [SMILES: CNCCC(Oc1cccc(-n2nc3c(N(C)C)nnc(C)c3c2C)c1)c1ccccc1OC] | 2-(3-(1-(2-methoxyphenyl)-3- (methylamino)propoxy)phenyl)- N,N,3,4-tetramethyl-2H-pyrazolo[3,4- d]pyridazin-7-amine (*) |
| 37 | 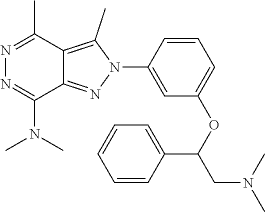 [SMILES: Cc1nnc(N(C)C)c2nn(-c3cccc(OC(CN(C)C)c4ccccc4)c3)c(C)c12] | 2-(3-(2-(dimethylamino)-1- phenylethoxy)phenyl)-N,N,3,4- tetramethyl-2H-pyrazolo[3,4- d]pyridazin-7-amine |
| 38 | 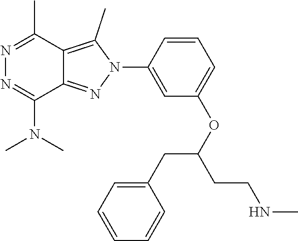 [SMILES: CNCCC(Cc1ccccc1)Oc1cccc(-n2nc3c(N(C)C)nnc(C)c3c2C)c1] | N,N,3,4-tetramethyl-2-(3-((4- (methylamino)-1-phenylbutan-2- yl)oxy)phenyl)-2H-pyrazolo[3,4- d]pyridazin-7-amine |
| 39 | 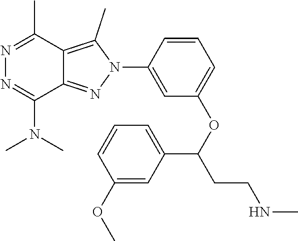 [SMILES: CNCCC(Oc1cccc(-n2nc3c(N(C)C)nnc(C)c3c2C)c1)c1cccc(OC)c1] | 2-(3-(1-(3-methoxyphenyl)-3- (methylamino)propoxy)phenyl)- N,N,3,4-tetramethyl-2H-pyrazolo[3,4- d]pyridazin-7-amine (*) |
| 40 | 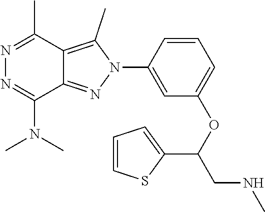 [SMILES: CNCC(Oc1cccc(-n2nc3c(N(C)C)nnc(C)c3c2C)c1)c1cccs1] | N,N,3,4-tetramethyl-2-(3-(2- (methylamino)-1-(thiophen-2- yl)ethoxy)phenyl)-2H-pyrazolo[3,4- d]pyridazin-7-amine (*) |
| 41 | 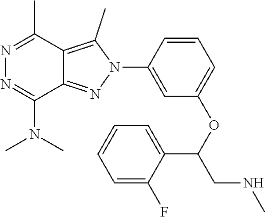 [SMILES: CNCC(Oc1cccc(-n2nc3c(N(C)C)nnc(C)c3c2C)c1)c1ccccc1F] | 2-(3-(1-(2-fluorophenyl)-2- (methylamino)ethoxy)phenyl)-N,N,3,4- tetramethyl-2H-pyrazolo[3,4- d]pyridazin-7-amine |
| 42 | 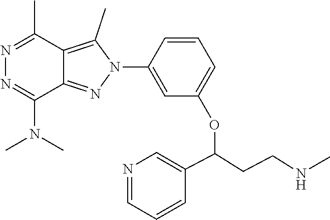 [SMILES: CNCCC(Oc1cccc(-n2nc3c(N(C)C)nnc(C)c3c2C)c1)c1cccnc1] | N,N,3,4-tetramethyl-2-(3-(3- (methylamino)-1-(pyridin-3- yl)propoxy)phenyl)-2H-pyrazolo[3,4- d]pyridazin-7-amine |
| 43 | 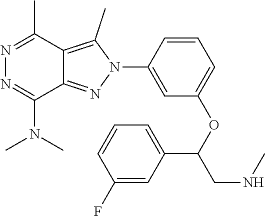 [SMILES: CNCC(Oc1cccc(-n2nc3c(N(C)C)nnc(C)c3c2C)c1)c1cccc(F)c1] | 2-(3-(1-(3-fluorophenyl)-2- (methylamino)ethoxy)phenyl)-N,N,3,4- tetramethyl-2H-pyrazolo[3,4- d]pyridazin-7-amine |
| 44 | 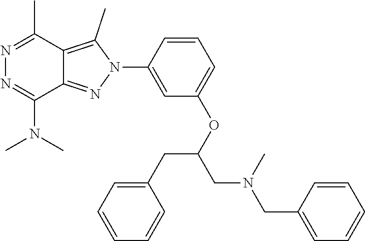 [SMILES: Cc1nnc(N(C)C)c2nn(-c3cccc(OC(Cc4ccccc4)CN(C)Cc4ccccc4)c3)c(C)c12] | 2-(3-((1-(benzyl(methyl)amino)-3- phenylpropan-2-yl)oxy)phenyl)- N,N,3,4-tetramethyl-2H-pyrazolo[3,4- d]pyridazin-7-amine |
| 45 | 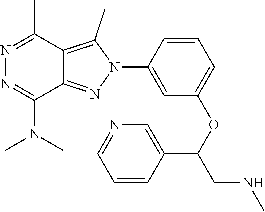 [SMILES: CNCC(Oc1cccc(-n2nc3c(N(C)C)nnc(C)c3c2C)c1)c1cccnc1] | N,N,3,4-tetramethyl-2-(3-(2- (methylamino)-1-(pyridin-3- yl)ethoxy)phenyl)-2H-pyrazolo[3,4- d]pyridazin-7-amine |
| 46 | 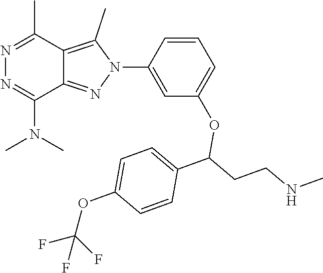 [SMILES: CNCCC(Oc1cccc(-n2nc3c(N(C)C)nnc(C)c3c2C)c1)c1ccc(OC(F)(F)F)cc1] | N,N,3,4-tetramethyl-2-(3-(3- (methylamino)-1-(4- (trifluoromethoxy)phenyl)propoxy) phenyl)-2H-pyrazolo[3,4-d]pyridazin- 7-amine |
| 47 | 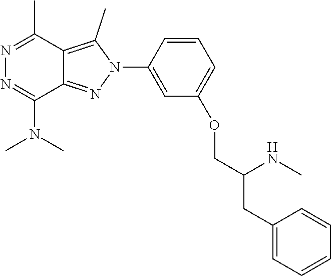 [SMILES: CNC(COc1cccc(-n2nc3c(N(C)C)nnc(C)c3c2C)c1)Cc1ccccc1] | N,N,3,4-tetramethyl-2-(3-(2- (methylamino)-3- phenylpropoxy)phenyl)-2H- pyrazolo[3,4-d]pyridazin-7-amine |
| 48 | 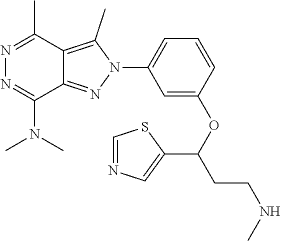 [SMILES: CNCCC(Oc1cccc(-n2nc3c(N(C)C)nnc(C)c3c2C)c1)c1cncs1] | N,N,3,4-tetramethyl-2-(3-(3- (methylamino)-1-(thiazol-5- yl)propoxy)phenyl)-2H-pyrazolo[3,4- d]pyridazin-7-amine |
| 49 | 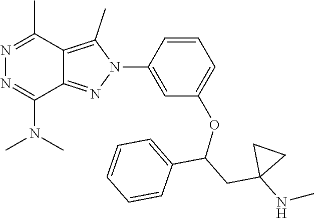 [SMILES: CNC1(CC(Oc2cccc(-n3nc4c(N(C)C)nnc(C)c4c3C)c2)c2ccccc2)CC1] | N,N,3,4-tetramethyl-2-(3-(2-(1- (methylamino)cyclopropyl)-1- phenylethoxy)phenyl)-2H-pyrazolo[3,4- d]pyridazin-7-amine |
| 50 | 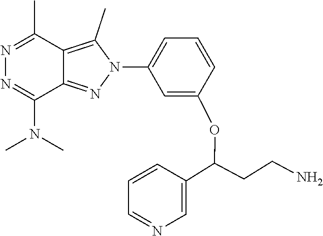 [SMILES: Cc1nnc(N(C)C)c2nn(-c3cccc(OC(CCN)c4cccnc4)c3)c(C)c12] | 2-(3-(3-amino-1-(pyridin-3- yl)propoxy)phenyl)-N,N,3,4- tetramethyl-2H-pyrazolo[3,4- d]pyridazin-7-amine |
| 51 | 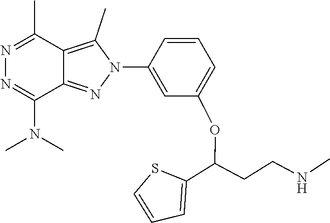 [SMILES: CNCCC(Oc1cccc(-n2nc3c(N(C)C)nnc(C)c3c2C)c1)c1cccs1] | N,N,3,4-Tetramethyl-2-(3-(3- (methylamino)-1-(thiophen-2- yl)propoxy)phenyl)-2H-pyrazolo[3,4- d]pyridazin-7-amine |
| 52 | 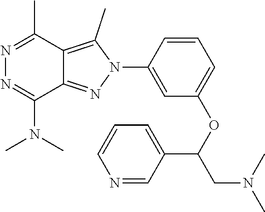 [SMILES: Cc1nnc(N(C)C)c2nn(-c3cccc(OC(CN(C)C)c4cccnc4)c3)c(C)c12] | 2-(3-(2-(Dimethylamino)-1-(pyridin-3- yl)ethoxy)phenyl)-N,N,3,4-tetramethyl- 2H-pyrazolo[3,4-d]pyridazin-7-amine |
| 53 | 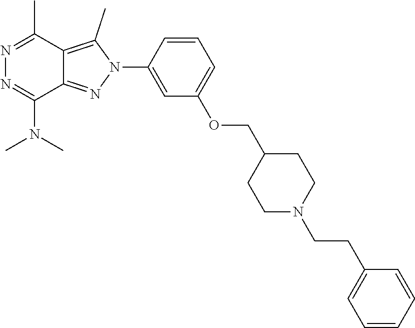 [SMILES: Cc1nnc(N(C)C)c2nn(-c3cccc(OCC4CCN(CCc5ccccc5)CC4)c3)c(C)c12] | N,N,3,4-Tetramethyl-2-(3-((1- phenethylpiperidin-4- yl)methoxy)phenyl)-2H-pyrazolo[3,4- d]pyridazin-7-amine |
| 54 | 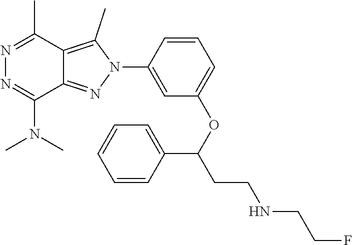 [SMILES: Cc1nnc(N(C)C)c2nn(-c3cccc(OC(CCNCCF)c4ccccc4)c3)c(C)c12] | 2-(3-(3-((2-fluoroethyl)amino)-1- phenylpropoxy)phenyl)-N,N,3,4- tetramethyl-2H-pyrazolo[3,4- d]pyridazin-7-amine |
| 55 | 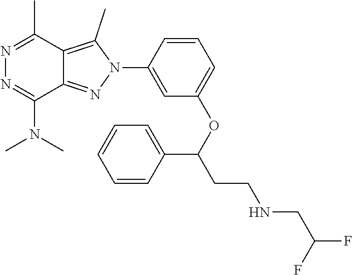 [SMILES: Cc1nnc(N(C)C)c2nn(-c3cccc(OC(CCNCC(F)F)c4ccccc4)c3)c(C)c12] | 2-(3-(3-((2,2-difluoroethyl)amino)-1- phenylpropoxy)phenyl)-N,N,3,4- tetramethyl-2H-pyrazolo[3,4- d]pyridazin-7-amine |
| 56 | 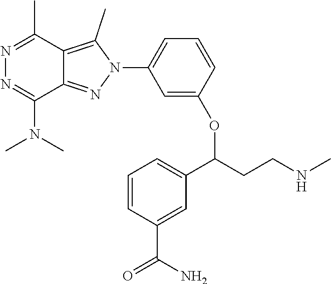 [SMILES: CNCCC(Oc1cccc(-n2nc3c(N(C)C)nnc(C)c3c2C)c1)c1cccc(C(N)=O)c1] | 3-(1-(3-(7-(Dimethylamino)-3,4- dimethyl-2H-pyrazolo[3,4-d]pyridazin- 2-yl)phenoxy)-3- (methylamino)propyl)benzamide |
| 57 | 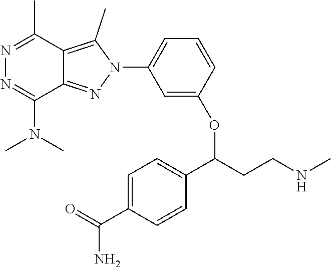 [SMILES: CNCCC(Oc1cccc(-n2nc3c(N(C)C)nnc(C)c3c2C)c1)c1ccc(C(N)=O)cc1] | 4-(1-(3-(7-(dimethylamino)-3,4- dimethyl-2H-pyrazolo[3,4-d]pyridazin- 2-yl)phenoxy)-3- (methylamino)propyl)benzamide |
| 58 | 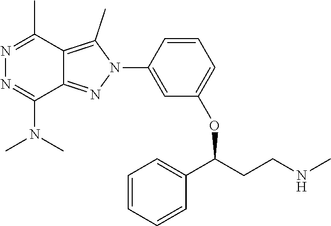 [SMILES: CNCC[C@H](Oc1cccc(-n2nc3c(N(C)C)nnc(C)c3c2C)c1)c1ccccc1] | (S)-N,N,3,4-Tetramethyl-2-(3-(3- (methylamino)-1- phenylpropoxy)phenyl)-2H- pyrazolo[3,4-d]pyridazin-7-amine |
| 59 | 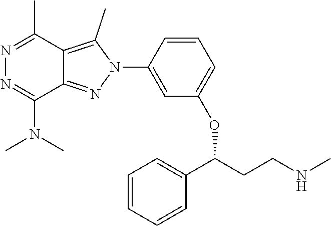 [SMILES: CNCC[C@@H](Oc1cccc(-n2nc3c(N(C)C)nnc(C)c3c2C)c1)c1ccccc1] | (R)-N,N,3,4-tetramethyl-2-(3-(3- (methylamino)-1- phenylpropoxy)phenyl)-2H- pyrazolo[3,4-d]pyridazin-7-amine |
| 60 | 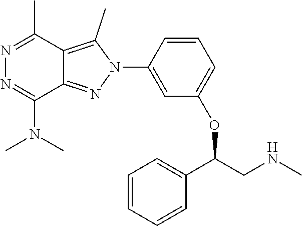 [SMILES: CNC[C@H](Oc1cccc(-n2nc3c(N(C)C)nnc(C)c3c2C)c1)c1ccccc1] | (R)-N,N,3,4-Tetramethyl-2-(3-(2- (methylamino)-1-phenylethoxy)phenyl)- 2H-pyrazolo[3,4-d]pyridazin-7-amine |
| 61 | 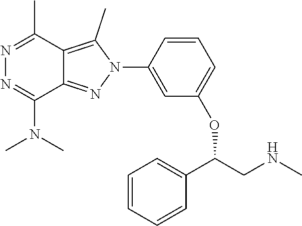 [SMILES: CNC[C@@H](Oc1cccc(-n2nc3c(N(C)C)nnc(C)c3c2C)c1)c1ccccc1] | (S)-N,N,3,4-tetramethyl-2-(3-(2- (methylamino)-1-phenylethoxy)phenyl)- 2H-pyrazolo[3,4-d]pyridazin-7-amine |
| 62 | 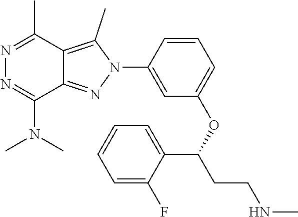 [SMILES: CNCC[C@@H](Oc1cccc(-n2nc3c(N(C)C)nnc(C)c3c2C)c1)c1ccccc1F] | (R)-2-(3-(1-(2-Fluorophenyl)-3- (methylamino)propoxy)phenyl)- N,N,3,4-tetramethyl-2H-pyrazolo[3,4- d]pyridazin-7-amine |
| 63 | 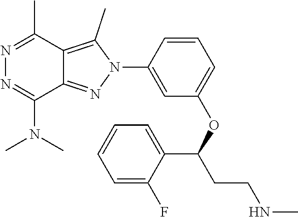 [SMILES: CNCC[C@H](Oc1cccc(-n2nc3c(N(C)C)nnc(C)c3c2C)c1)c1ccccc1F] | (S)-2-(3-(1-(2-fluorophenyl)-3- (methylamino)propoxy)phenyl)- N,N,3,4-tetramethyl-2H-pyrazolo[3,4- d]pyridazin-7-amine |
| 64 | 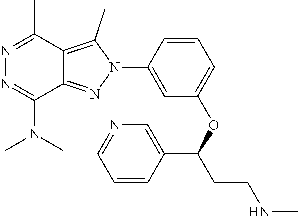 [SMILES: CNCC[C@H](Oc1cccc(-n2nc3c(N(C)C)nnc(C)c3c2C)c1)c1cccnc1] | (S)-N,N,3,4-tetramethyl-2-(3-(3- (methylamino)-1-(pyridin-3- yl)propoxy)phenyl)-2H-pyrazolo[3,4- d]pyridazin-7-amine |
| 65 | 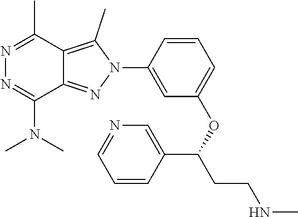 [SMILES: CNCC[C@@H](Oc1cccc(-n2nc3c(N(C)C)nnc(C)c3c2C)c1)c1cccnc1] | (R)-N,N,3,4-tetramethyl-2-(3-(3- (methylamino)-1-(pyridin-3- yl)propoxy)phenyl)-2H-pyrazolo[3,4- d]pyridazin-7-amine |

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred further embodiment, the compounds of the general Formula (I) are selected from

| EX | Structure | Chemical name |
|---|---|---|
| 66 | 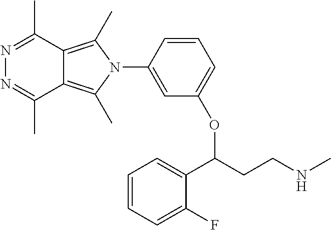 [SMILES: CNCCC(Oc1cccc(-n2c(C)c3c(C)nnc(C)c3c2C)c1)c1ccccc1F] | 3-(2-fluorophenyl)-N-methyl-3-(3- (1,4,5,7-tetramethyl-6H-pyrrolo[3,4- d]pyridazin-6-yl)phenoxy)propan-1- amine |
| 67 | 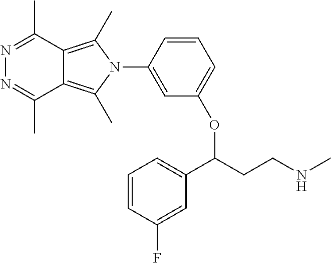 [SMILES: CNCCC(Oc1cccc(-n2c(C)c3c(C)nnc(C)c3c2C)c1)c1cccc(F)c1] | 3-(3-fluorophenyl)-N-methyl-3-(3- (1,4,5,7-tetramethyl-6H-pyrrolo[3,4- d]pyridazin-6-yl)phenoxy)propan-1- amine |
| 68 | 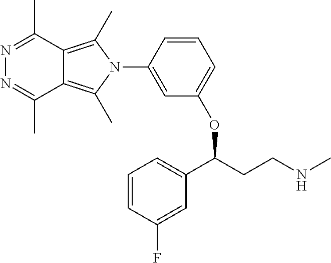 [SMILES: CNCC[C@H](Oc1cccc(-n2c(C)c3c(C)nnc(C)c3c2C)c1)c1cccc(F)c1] | (S)-3-(3-Fluorophenyl)-N-methyl-3-(3- (1,4,5,7-tetramethyl-6H-pyrrolo[3,4- d]pyridazin-6-yl)phenoxy)propan-1- amine |
| 69 | 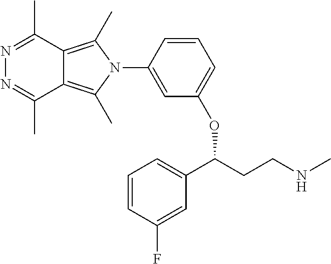 [SMILES: CNCC[C@@H](Oc1cccc(-n2c(C)c3c(C)nnc(C)c3c2C)c1)c1cccc(F)c1] | (R)-3-(3-fluorophenyl)-N-methyl-3-(3- (1,4,5,7-tetramethyl-6H-pyrrolo[3,4- d]pyridazin-6-yl)phenoxy)propan-1- amine |

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),
Rₓ is selected from substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
R_{x′} is selected from hydrogen, substituted or unsubstituted C₁₋₆ alkyl, substituted or unsubstituted C₂₋₆ alkenyl and substituted or unsubstituted C₂₋₆ alkynyl;
   the alkyl, alkenyl or alkynyl in R_{X} or R_{X′}, if substituted, is substituted with one or more substituent/s selected from —OR₉, halogen, —CN, haloalkyl, haloalkoxy, and —NR₉R_{9′};
wherein R₉ and R_{9′} are independently selected from hydrogen, unsubstituted C₁₋₆ alkyl, unsubstituted C₂₋₆ alkenyl, and unsubstituted C₂₋₆ alkynyl;
   the aryl, heterocyclyl or cycloalkyl, also in alkylaryl, alkylcycloalkyl and alkylheterocyclyl, in R_{X}, if substituted, is substituted with one or more substituent/s selected from halogen, —R₁₀, —OR₁₀, —NO₂, —NR₁₀R_{10′}, —NR₁₀C(O)R_{10′}, —NR₁₀S(O)₂R_{10′}, —S(O)₂NR₁₀R_{10′}, —NR₁₀C(O)NR_{10′}R_{10″}, —SR₁₀, —S(O)R₁₀, S(O)₂R₁₀, —CN, haloalkyl, haloalkoxy, —C(O)OR₁₀, —C(O)NR₁₀R_{10″}, —OCH₂CH₂OR₁₀, —NR₁₀S(O)₂NR_{10′}R_{10″} and C(CH₃)₂OR₁₀;
wherein R₁₀, R_{10′} and R_{10″} are independently selected from hydrogen, unsubstituted C₁₋₆ alkyl, unsubstituted C₂₋₆ alkenyl, unsubstituted C₂₋₆ alkynyl, unsubstituted aryl, unsubstituted alkylaryl, unsubstituted cycloalkyl and unsubstituted alkylcycloalkyl, unsubstituted heterocyclyl and unsubstituted alkyheterocyclyl;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),

R₁ is selected from —NR₆R_{6′} and substituted or unsubstituted N-containing-heterocyclyl;
wherein R₆ and R_{6′} are independently selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
wherein said cycloalkyl, aryl or heterocyclyl in R₁ or R₆, also in alkylaryl, alkylcycloalkyl and alkylheterocyclyl, if substituted, is substituted with one or more substituent/s selected from halogen, —R₁₁, —OR₁₁, —NO₂, —NR₁₁R₁₁, NR₁₁C(O)R_{11′}, —NR₁₁S(O)₂R_{11′}, —S(O)₂NR₁₁R_{11′}, —NR₁₁C(O)NR_{11′}R_{11″}, —SR₁₁, —S(O)R₁₁, S(O)₂R₁₁, —CN, haloalkyl, haloalkoxy, —C(O)OR₁₁, —C(O)NR₁₁R_{11′}, —OCH₂CH₂OH, —NR₁₁S(O)₂NR_{11′}R_{11″}, C(CH₃)₂OR₁₁, substituted or unsubstituted alkylaryl, substituted or unsubstituted alkylcycloalkyl and substituted or unsubstituted alkyheterocyclyl;
wherein the alkyl, alkenyl or alkynyl in R₆ or R_{6′}, if substituted, is substituted with one or more substituent/s selected from —OR₁₁, halogen, —CN, haloalkyl, haloalkoxy and —NR₁₁R_{11′};
wherein R₁₁, R_{11′} and R_{11″} are independently selected from hydrogen, unsubstituted C₁₋₆ alkyl, unsubstituted C₂₋₆ alkenyl and unsubstituted C₂₋₆ alkynyl;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),

R₂ is selected from hydrogen, —NR₇R_{7′}, —CN, —CHR₇R_{7′} and substituted or unsubstituted heterocyclyl;
wherein R₇ and R_{7′} are independently selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
wherein said cycloalkyl, aryl or heterocyclyl in R₂, R₇ or R_{7′}, also in alkylaryl, alkylcycloalkyl and alkylheterocyclyl, if substituted, is substituted with one or more substituent/s selected from halogen, —R₁₂, —OR₁₂, —NO₂, —NR₁₂R_{12′}, NR₁₂C(O)R_{12′}, —NR₁₂S(O)₂R_{12′}, —S(O)₂NR₁₂R_{12′}, —NR₁₂C(O)NR_{12′}R_{12″}, —SR₁₂, —S(O)R₁₂, S(O)₂R₁₂, —CN, haloalkyl, haloalkoxy, —C(O)OR₁₂, —C(O)NR₁₂R_{12′}, —OCH₂CH₂OR₁₂, NR₁₂S(O)₂NR_{12′}R_{12″} and —C(CH₃)₂OR₁₂;
wherein the alkyl, alkenyl or alkynyl in R₇ or R_{7′}, if substituted, is substituted with one or more substituent/s selected from —OR₁₂, halogen, —CN, haloalkyl, haloalkoxy and —NR₁₂R_{12′};
wherein R₁₂, R_{12′} and R_{12″} are independently selected from hydrogen, unsubstituted C₁₋₆ alkyl, and unsubstituted C₂₋₆ alkenyl and unsubstituted C₂₋₆ alkynyl;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment of the invention the compound of general Formula (I),

the alkyl, alkenyl or alkynyl, other than those defined in Rₓ, R_{x′}, R₆, R_{6′}, R₇ or R_{7′}, if substituted, is substituted with one or more substituent/s selected from —OR₁₃, halogen, —CN, haloalkyl, haloalkoxy and —NR₁₃R_{13′};
wherein R₁₃ and R_{13′} are independently selected from hydrogen, unsubstituted C₁₋₆ alkyl, unsubstituted C₂₋₆ alkenyl, and unsubstituted C₂₋₆ alkynyl;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment of the invention the compound of general Formula (I),

the aryl, heterocyclyl or cycloalkyl, also in alkylaryl, alkylcycloalkyl and alkylheterocyclyl, other than those defined in Rₓ, R_{x′}, R₁, R₂, R₆, R_{6′}, R₇ or R_{7′}, if substituted, is substituted with one or more substituent/s selected from halogen, —R₁₄, —OR₁₄, —NO₂, —NR₁₄R_{14′}, NR₁₄C(O)R_{14′}, —NR₁₄S(O)₂R_{14′}, —S(O)₂NR₁₄R_{14′}, —NR₁₄C(O)NR_{14′}R_{14″}, —SR₁₄, —S(O)R₁₄, S(O)₂R₁₄, —CN, haloalkyl, haloalkoxy, —C(O)OR₁₄, —C(O)NR₁₄R_{14′}, —OCH₂CH₂OR₁₄, —NR₁₄S(O)₂NR_{14′}R_{14″} and C(CH₃)₂OR₁₄;
wherein R₁₄, R_{14′} and R_{14″} are independently selected from hydrogen, unsubstituted C₁₋₆ alkyl, unsubstituted C₂₋₆ alkenyl, unsubstituted C₂₋₆ alkynyl, unsubstituted aryl, unsubstituted alkylaryl, unsubstituted cycloalkyl and unsubstituted alkylcycloalkyl, unsubstituted heterocyclyl and unsubstituted alkyheterocyclyl;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),

the alkyl, alkenyl or alkynyl in R_{X} or R_{X′}, if substituted, is substituted with one or more substituent/s selected from —OR₉, halogen, —CN, haloalkyl, haloalkoxy, and —NR₉R_{9′};
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),

the aryl, heterocyclyl or cycloalkyl, also in alkylaryl, alkylcycloalkyl and alkylheterocyclyl, in R_{X}, if substituted, is substituted with one or more substituent/s selected from halogen, —R₁₀, —OR₁₀, —NO₂, —NR₁₀R_{10′}, —NR₁₀C(O)R_{10′}, —NR₁₀S(O)₂R_{10′}, —S(O)₂NR₁₀R_{10′}, —NR₁₀C(O)NR_{10′}R_{10″}, —SR₁₀, —S(O)R₁₀, S(O)₂R₁₀, —CN, haloalkyl, haloalkoxy, —C(O)OR₁₀, —C(O)NR₁₀R_{10′}, —OCH₂CH₂OR₁₀, —NR₁₀S(O)₂NR_{10′}R_{10″} and C(CH₃)₂OR₁₀;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),

the aryl, heterocyclyl or cycloalkyl, also in alkylaryl, alkylcycloalkyl and alkylheterocyclyl, in R_{X}, if substituted, is substituted with one or more substituent/s selected from halogen, —OR₁₀, —CN, haloalkoxy and —C(O)NR₁₀R_{10′};
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),

the aryl, heterocyclyl or cycloalkyl, also in alkylaryl, alkylcycloalkyl and alkylheterocyclyl, in R_{X}, if substituted, is substituted with one or more substituent/s selected from halogen, —OR₁₀, —CN, haloalkoxy and —C(O)NR₁₀R_{10′};
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),
the cycloalkyl, aryl or heterocyclyl in R₁ or R₆, also in alkylaryl, alkylcycloalkyl and alkylheterocyclyl, if substituted, is substituted with one or more substituent/s selected from halogen, —R₁₁, —OR₁₁, —NO₂, —NR₁₁R_{11′}, NR₁₁C(O)R_{11′}, —NR₁₁S(O)₂R_{11′}, —S(O)₂NR₁₁R_{11′}, —NR₁₁C(O)NR_{11′}R_{11″}, —SR₁₁, —S(O)R₁₁, S(O)₂R₁₁, —CN, haloalkyl, haloalkoxy, —C(O)OR₁₁, —C(O)NR₁₁R_{11′}, —OCH₂CH₂OH, —NR₁₁S(O)₂NR_{11′}R_{11″}, C(CH₃)₂OR₁₁, substituted or unsubstituted alkylaryl, substituted or unsubstituted alkylcycloalkyl and substituted or unsubstituted alkyheterocyclyl;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),
the cycloalkyl, aryl or heterocyclyl in R₁, also in alkylaryl, alkylcycloalkyl and alkylheterocyclyl, if substituted, is substituted with one or more substituted or unsubstituted alkylaryl;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),

the alkyl, alkenyl or alkynyl in R₆ or R_{6′}, if substituted, is substituted with one or more substituent/s selected from —OR₁₁, halogen, —CN, haloalkyl, haloalkoxy and —NR₁₁R_{11′};
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),

the cycloalkyl, aryl or heterocyclyl in R₂, R₇ or R_{7′}, also in alkylaryl, alkylcycloalkyl and alkylheterocyclyl, if substituted, is substituted with one or more substituent/s selected from halogen, —R₁₂, —OR₁₂, —NO₂, —NR₁₂R_{12′}, NR₁₂C(O)R_{12′}, —NR₁₂S(O)₂R_{12′}, —S(O)₂NR₁₂R_{12′}, —NR₁₂C(O)NR_{12′}R_{12″}, —SR₁₂, —S(O)R₁₂, S(O)₂R₁₂, —CN, haloalkyl, haloalkoxy, —C(O)OR₁₂, —C(O)NR₁₂R_{12′}, —OCH₂CH₂OR₁₂, —NR₁₂S(O)₂NR_{12′}R_{12″} and —C(CH₃)₂OR₁₂;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),

the alkyl, alkenyl or alkynyl in R₇ or R_{7′}, if substituted, is substituted with one or more substituent/s selected from —OR₁₂, halogen, —CN, haloalkyl, haloalkoxy and —NR₁₂R_{12′};
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),

the alkyl, alkenyl or alkynyl in R₇ or R_{7′}, if substituted, is substituted with one or more substituent/s selected —OR₁₂;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),

the alkyl, alkenyl or alkynyl, other than those defined in Rₓ, R_{x′}, R₆, R_{6′}, R₇ or R_{7′}, if substituted, is substituted with one or more substituent/s selected from —OR₁₃, halogen, —CN, haloalkyl, haloalkoxy and —NR₁₃R_{13′};
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),

the aryl, heterocyclyl or cycloalkyl, also in alkylaryl, alkylcycloalkyl and alkylheterocyclyl, other than those defined in Rₓ, R_{x′}, R₁, R₂, R₆, R_{6′}, R₇ or R_{7′}, if substituted, is substituted with one or more substituent/s selected from halogen, —R₁₄, —OR₁₄, —NO₂, —NR₁₄R_{14′}, NR₁₄C(O)R_{14′}, —NR₁₄S(O)₂R_{14′}, —S(O)₂NR₁₄R_{14′}, —NR₁₄C(O)NR_{14′}R_{14″}, —SR₁₄, —S(O)R₁₄, S(O)₂R₁₄, —CN, haloalkyl, haloalkoxy, —C(O)OR₁₄, —C(O)NR₁₄R_{14′}, —OCH₂CH₂OR₁₄, —NR₁₄S(O)₂NR_{14′}R_{14″} and C(CH₃)₂OR₁₄;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In an embodiment of the compound according to the invention of general Formula (I),

the halogen is fluorine, chlorine, iodine or bromine;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a most preferred embodiment of the compound according to the invention of general Formula (I)

the halogen is fluorine;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In an embodiment of the compound according to the invention of general Formula (I),

the haloalkyl is —CF3;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment of the compound according to the invention of general Formula (I),

the haloalkoxy is —OCF3;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

As this invention is aimed at providing a compound or a chemically related series of compounds which act as dual ligands of the α2δ subunit, particularly the α2δ-1 subunit, of the voltage-gated calcium channel and the NET receptor it is a very preferred embodiment in which the compounds are selected which act as dual ligands of the α2δ subunit, particularly the α2δ-1 ubunit, of the voltage-gated calcium channel and the NET receptor and especially compounds which have a binding expressed as Kᵢ responding to the following scales:

Kᵢ(NET) is preferably <1000 nM, more preferably <500 nM, even more preferably <100 nM.
Kᵢ(α2δ1) is preferably <10000 nM, more preferably <5000 nM, even more preferably <500 nM.

In the following the phrase “compound of the invention” is used. This is to be understood as any compound according to the invention as described above according to general Formula (I), (I′), (I²′), (I³′) and (IZ).

The compounds of the invention represented by the above described Formula (I) may include enantiomers depending on the presence of chiral centres or isomers depending on the presence of multiple bonds (e.g. Z, E). The single isomers, enantiomers or diastereoisomers and mixtures thereof fall within the scope of the present invention.

For the sake of clarity the expression “a compound according to Formula (I), wherein e.g. R₁, R₂, R₃, R₄, R₅, R_{5′}, R_{c}, R_{c′}, X, W, m, n and p are as defined below in the detailed description” would (just like the expression “a compound of Formula (I) as defined in any one of claims e.g. 1 to 8” found in the claims) refer to “a compound according to Formula (I)”, wherein the definitions of the respective substituents R₁ etc. (also from the cited claims) are applied. In addition, this would also mean, though (especially in regards to the claims) that also one or more disclaimers defined in the description (or used in any of the cited claims like e.g. claim 1) would be applicable to define the respective compound. Thus, a disclaimer found in e.g. claim 1 would be also used to define the compound “of Formula (I) as defined in any one of the corresponding related claims e.g. 1 to 8”.

In general the processes are described below in the experimental part. The starting materials are commercially available or can be prepared by conventional methods.

A preferred aspect of the invention is also a process for the production of a compound according to Formula (I), following scheme 1, scheme 2 or scheme 3.

A preferred embodiment of the invention is a process for the production of a compound according to Formula (I), wherein, if not defined otherwise, m, n, p, R₁, R₂, R₃, R₄, R_{4′}, R₅, R_{5′}, R_{c}, R_{c′}, W and X have the meanings defined in the description.

In a particular embodiment there is a process for the production of a compound according to Formula (I),

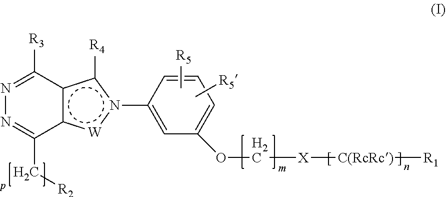

wherein when the group [CH₂]ₚR₂ is attached to the core structure through a carbon atom, said process comprises treating a compound of formula III

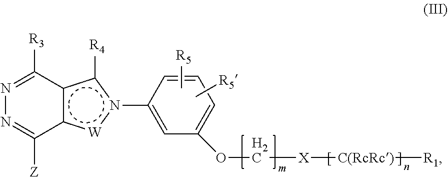

wherein Z represents a halogen, preferably chloro, or triflate with a suitable organometallic reagent of formula IVa,

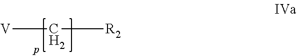

wherein V represents a suitable organometallic reagent, preferably a boron or zinc reagent.

In a particular embodiment there is a process for the production of a compound according to Formula (I),

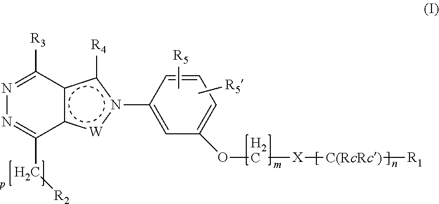

wherein when the group [CH₂]ₚR₂ is —NR₇R_{7′}, said process comprises treating a compound of formula III

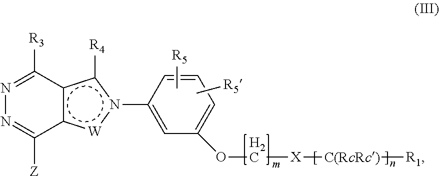

wherein Z represents a halogen, preferably chloro, or triflate with an amine of formula IVb

HNR₇R_{7′} IVb.

In a particular embodiment there is a process for the production of a compound according to Formula (I),

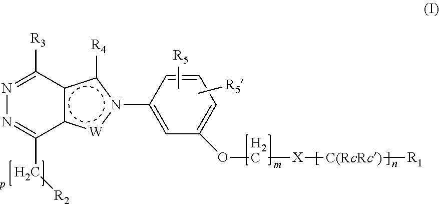

said process comprises treating a compound of formula VH

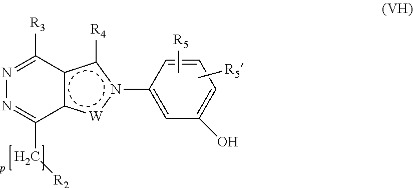

with a compound of formula VI

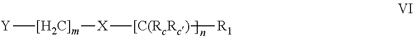

wherein Y represents a leaving group, such as halogen, mesylate, tosylate, nosylate or triflate, or OH.

In a particular embodiment there is a process for the production of a compound according to Formula (III), (IIIP) or (IIIH),

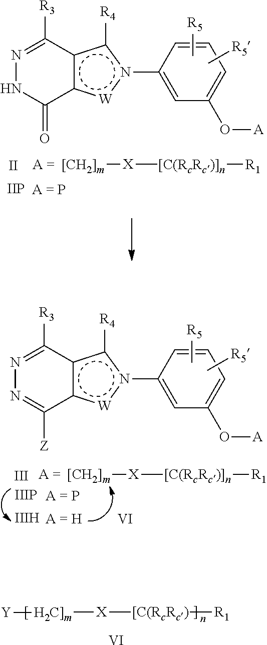

wherein P represents a suitable protecting group, such as alkyl or benzyl, preferably methyl, Y represents a leaving group, such as halogen, mesylate, tosylate, nosylate or triflate, or OH, and Z represents an halogen, preferably chloro, or triflate, following the method described for scheme 1.

In a particular embodiment there is a process for the production of a compound according to Formula (I), (VP) or (VH),

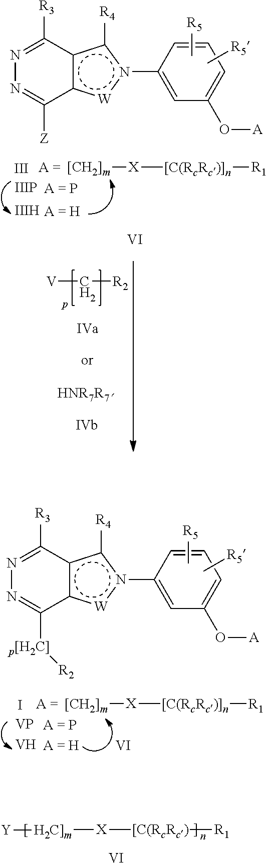

wherein P represents a suitable protecting group, such as alkyl or benzyl, preferably methyl, Y represents a leaving group, such as halogen, mesylate, tosylate, nosylate or triflate, or OH, and Z represents an halogen, preferably chloro, or triflate, following the method described for scheme 1.

In a particular embodiment there is a process for the production of a compound according to Formula (IX) or (IXP),

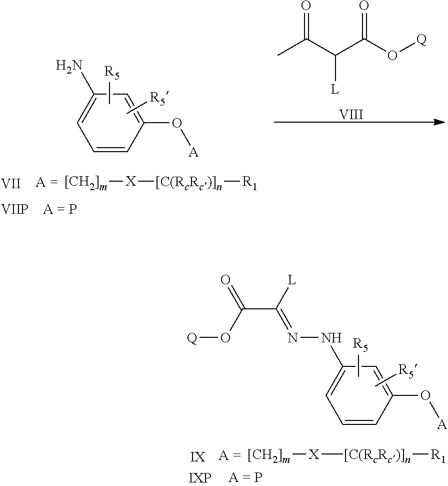

wherein P represents a suitable protecting group, such as alkyl or benzyl, preferably methyl, and L represents halogen, like fluorine, chlorine, bromine or iodine, following the method described for scheme 2.

In a particular embodiment there is a process for the production of a compound according to Formula (XI) or (XIP),

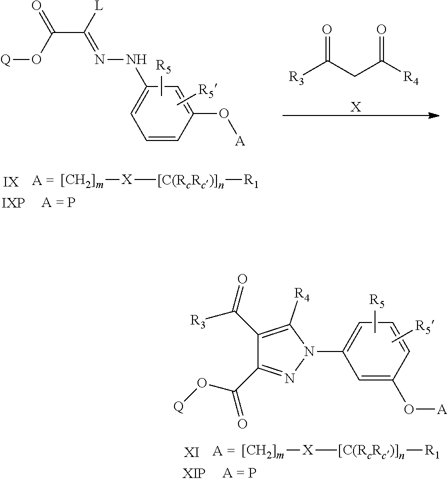

wherein P represents a suitable protecting group, such as alkyl or benzyl, preferably methyl, and Q represents an alkyl group, preferably methyl or ethyl, following the method described for scheme 2.

In a particular embodiment there is a process for the production of a compound according to Formula (IIa) or (IIaP),

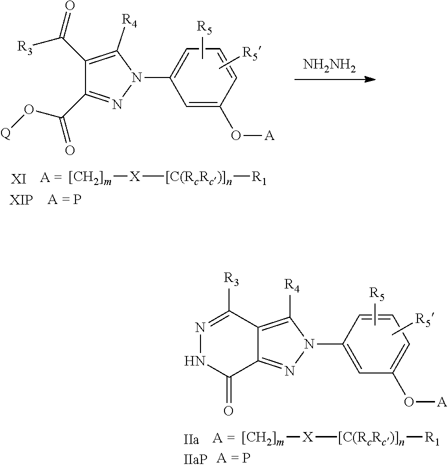

wherein P represents a suitable protecting group, such as alkyl or benzyl, preferably methyl, and Q represents an alkyl group, preferably methyl or ethyl, following the method described for scheme 2.

In a particular embodiment there is a process for the production of a compound according to Formula (XIII) or (XIIIP),

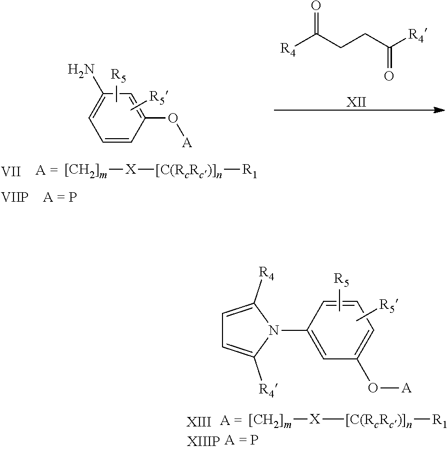

wherein P represents a suitable protecting group, such as alkyl or benzyl, preferably methyl, following the method described for scheme 3.

In a particular embodiment there is a process for the production of a compound according to Formula (XV) or (XVP),

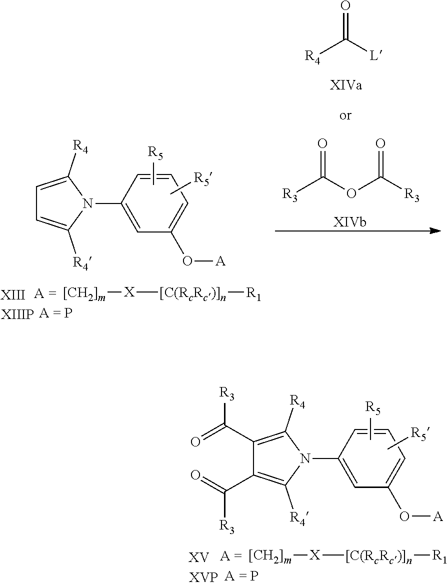

wherein P represents a suitable protecting group, such as alkyl or benzyl, preferably methyl, and L′ represents halogen, like fluorine, chlorine, bromine or iodine, following the method described for scheme 3.

In a particular embodiment there is a process for the production of a compound according to Formula (Ib), (VbP) or (VbH),

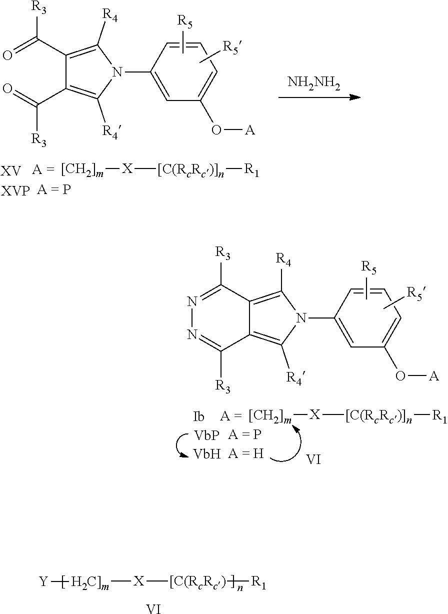

wherein P represents a suitable protecting group, such as alkyl or benzyl, preferably methyl, Y represents a leaving group, such as halogen, mesylate, tosylate, nosylate or triflate, or OH, following the method described for scheme 3.

In a particular embodiment there is a process for the production of a compound according to Formula (I), by the reduction reaction of a carbonyl derivative with a suitable reductive reagent, preferably sodium borohydride, in an organic solvent, preferably MeOH, to afford a hydroxyl compound.

In a particular embodiment there is a process for the production of a compound according to Formula (I), by deprotection reaction of a compound of formula I that contains an amine protecting group such as a carbamate, preferably tert-butoxy carbonyl, by any suitable method, such as treatment with an acid, preferably HCl or trifluoroacetic acid in an appropriate solvent such as 1,4-dioxane, DCM, ethyl acetate or a mixture of an organic solvent and water.

In a particular embodiment there is a process for the production of a compound according to Formula (I), by reductive amination reaction of a compound of formula I that contains an amino group with an aldehyde, preferably carried out with a reductive reagent, preferably sodium triacetoxyborohydride, in an organic solvent, preferably DCE, in the presence of an organic base, preferably DIPEA or TEA. Alternatively, the reaction can be carried out in the presence of an acid, preferably acetic acid.

In a particular embodiment there is a process for the production of a compound according to Formula (I), by reaction of a compound of formula I that contains an amino group with an alkylating reagent, in the presence of a base, preferably DIPEA or K₂CO₃, in an organic solvent, preferably acetonitrile, at suitable temperature, such as in the range of 0-120° C.

In a particular embodiment there is a process for the production of a compound according to Formula (I), by reaction of a compound of formula I that contains an amino group with a vinyl derivative, in an organic solvent, preferably 2-methoxyethanol, at suitable temperature, such as in the range of 20-140° C.

In a particular embodiment a compound of Formula (II),

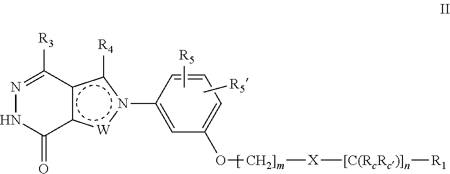

is used for the preparation of compounds of Formula (I).

In a particular embodiment a compound of Formula (IIIP),

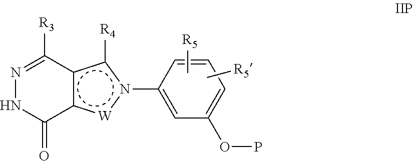

wherein P is a suitable protecting group, such as alkyl or benzyl, preferably methyl, is used for the preparation of compounds of Formula (I).

In a particular embodiment a compound of Formula (III),

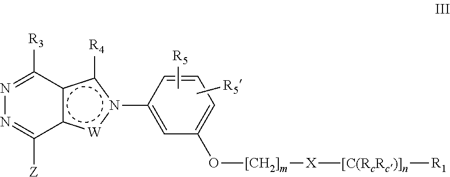

is used for the preparation of compounds of Formula (I).

In a particular embodiment a compound of Formula (IIIP),

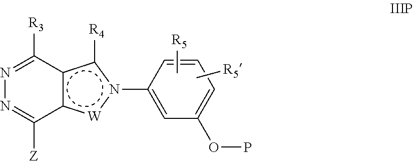

wherein P is a suitable protecting group, such as alkyl or benzyl, preferably methyl, and Z represents an halogen, preferably chloro, or triflate, is used for the preparation of compounds of Formula (I).

In a particular embodiment a compound of Formula (IIIH),

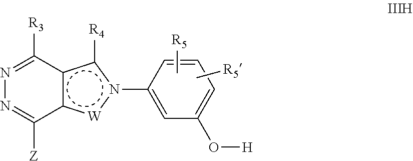

wherein Z represents an halogen, preferably chloro, or triflate, is used for the preparation of compounds of Formula (I).

In a particular embodiment a compound of Formula (IVa),

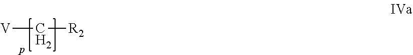

wherein V represents a suitable organometallic reagent, preferably a boron or zinc reagent, is used for the preparation of compounds of Formula (I).

In a particular embodiment a compound of Formula (IVb),

HNR₇R_{7′} IVb

is used for the preparation of compounds of Formula (I).

In a particular embodiment a compound of Formula (VP),

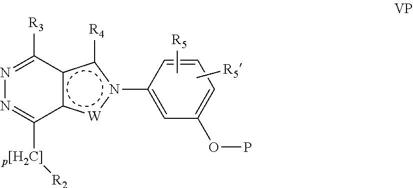

wherein P is a suitable protecting group, such as alkyl or benzyl, preferably methyl, is used for the preparation of compounds of Formula (I).

In a particular embodiment a compound of Formula (VH),

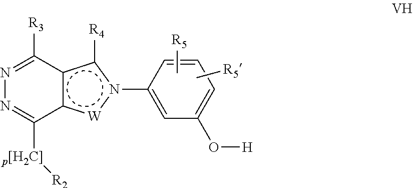

is used for the preparation of compounds of Formula (I).

In a particular embodiment a compound of Formula (VI),

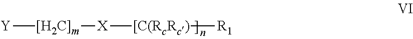

wherein Y is a leaving group, such as halogen, mesylate, tosylate, nosylate or triflate, or OH, is used for the preparation of compounds of Formula (I).

In a particular embodiment a compound of Formula (VII),

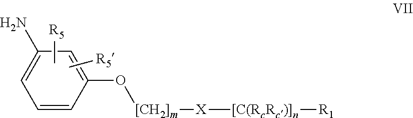

is used for the preparation of compounds of Formula (I).

In a particular embodiment a compound of Formula (VIIP),

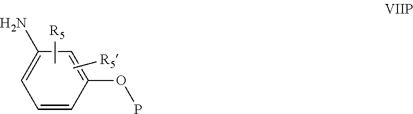

wherein P is a suitable protecting group, such as alkyl or benzyl, preferably methyl, is used for the preparation of compounds of Formula (I).

In a particular embodiment a compound of Formula (VIII),

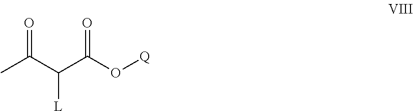

wherein Q is an alkyl group, preferably methyl or ethyl, is used for the preparation of compounds of Formula (I).

In a particular embodiment a compound of Formula (IX),

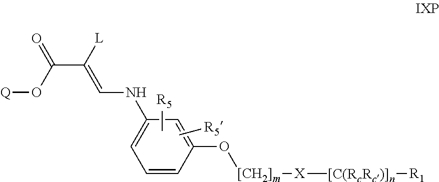

wherein L represents halogen, like fluorine, bromine, iodine or chlorine, is used for the preparation of compounds of Formula (I).

In a particular embodiment a compound of Formula (IXP),

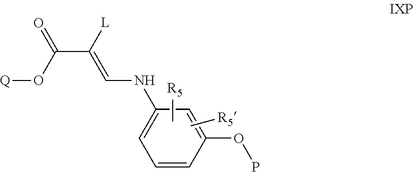

wherein P is a suitable protecting group, such as alkyl or benzyl, preferably methyl, Q is an alkyl group, preferably methyl or ethyl and L represents halogen, like fluorine, bromine, iodine or chlorine, is used for the preparation of compounds of Formula (I).

In a particular embodiment a compound of Formula (X),

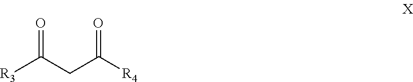

is used for the preparation of compounds of Formula (I).

In a particular embodiment a compound of Formula (XI),

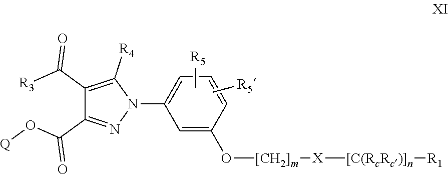

wherein Q is an alkyl group, preferably methyl or ethyl, is used for the preparation of compounds of Formula (I).

In a particular embodiment a compound of Formula (XIP),

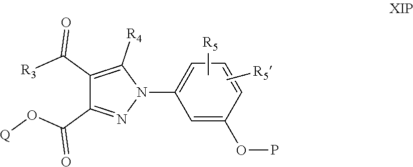

wherein P is a suitable protecting group, such as alkyl or benzyl, preferably methyl and Q is an alkyl group, preferably methyl or ethyl, is used for the preparation of compounds of Formula (I).

In a particular embodiment a compound of Formula (IIa),

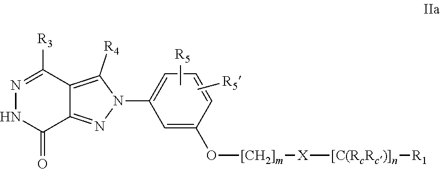

is used for the preparation of compounds of Formula (I).

In a particular embodiment a compound of Formula (IIaP),

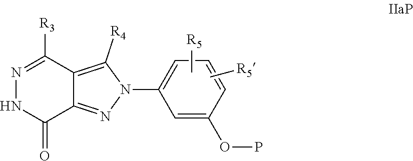

wherein P is a suitable protecting group, such as alkyl or benzyl, preferably methyl, is used for the preparation of compounds of Formula (I).

In a particular embodiment a compound of Formula (XII),

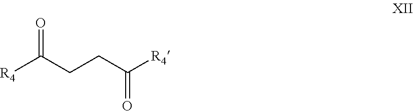

is used for the preparation of compounds of Formula (I).

In a particular embodiment a compound of Formula (XIII),

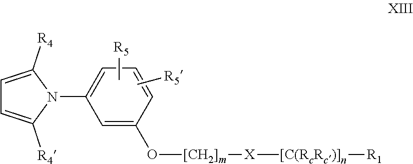

is used for the preparation of compounds of Formula (I).

In a particular embodiment a compound of Formula (XIIIP),

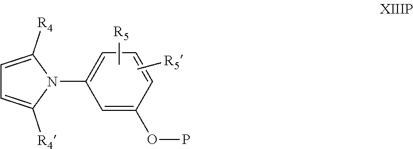

wherein P is a suitable protecting group, such as alkyl or benzyl, preferably methyl, is used for the preparation of compounds of Formula (I).

In a particular embodiment a compound of Formula (XIVa),

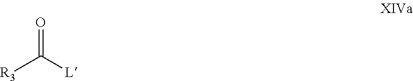

wherein L′ represents halogen, like fluorine, bromine, iodine or chlorine, is used for the preparation of compounds of Formula (I).

In a particular embodiment a compound of Formula (XIVb),

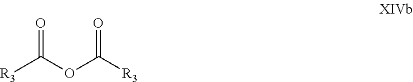

is used for the preparation of compounds of Formula (I).

In a particular embodiment a compound of Formula (XV),

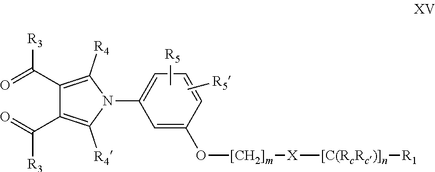

is used for the preparation of compounds of Formula (I).

In a particular embodiment a compound of Formula (XVP),

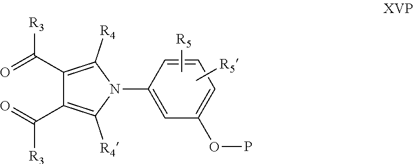

wherein P is a suitable protecting group, such as alkyl or benzyl, preferably methyl, is used for the preparation of compounds of Formula (I).

In a particular embodiment a compound of Formula (Ib),

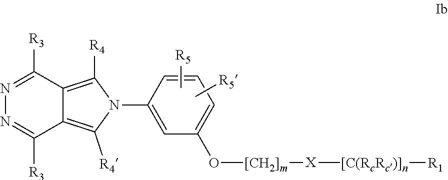

is used for the preparation of compounds of Formula (I).

In a particular embodiment a compound of Formula (VbP),

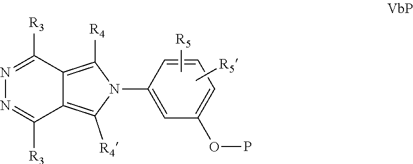

wherein P is a suitable protecting group, such as alkyl or benzyl, preferably methyl, is used for the preparation of compounds of Formula (I).

In a particular embodiment a compound of Formula (VbH),

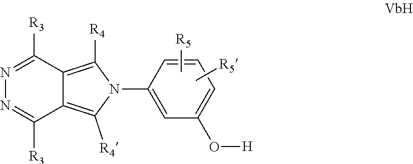

is used for the preparation of compounds of Formula (I).

The obtained reaction products may, if desired, be purified by conventional methods, such as crystallisation and chromatography. Where the above described processes for the preparation of compounds of the invention give rise to mixtures of stereoisomers, these isomers may be separated by conventional techniques such as preparative chromatography. If there are chiral centers the compounds may be prepared in racemic form, or individual enantiomers may be prepared either by enantiospecific synthesis or by resolution.

One preferred pharmaceutically acceptable form of a compound of the invention is the crystalline form, including such form in pharmaceutical composition. In the case of salts and also solvates of the compounds of the invention the additional ionic and solvent moieties must also be non-toxic. The compounds of the invention may present different polymorphic forms, it is intended that the invention encompasses all such forms.

Another aspect of the invention refers to a pharmaceutical composition which comprises a compound according to the invention as described above according to general formula I or a pharmaceutically acceptable salt or stereoisomer thereof, and a pharmaceutically acceptable carrier, adjuvant or vehicle. The present invention thus provides pharmaceutical compositions comprising a compound of this invention, or a pharmaceutically acceptable salt or stereoisomers thereof together with a pharmaceutically acceptable carrier, adjuvant, or vehicle, for administration to a patient.

Examples of pharmaceutical compositions include any solid (tablets, pills, capsules, granules etc.) or liquid (solutions, suspensions or emulsions) composition for oral, topical or parenteral administration.

In a preferred embodiment the pharmaceutical compositions are in oral form, either solid or liquid. Suitable dose forms for oral administration may be tablets, capsules, syrops or solutions and may contain conventional excipients known in the art such as binding agents, for example syrup, acacia, gelatin, sorbitol, tragacanth, or polyvinylpyrrolidone; fillers, for example lactose, sugar, maize starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulfate.

The solid oral compositions may be prepared by conventional methods of blending, filling or tabletting. Repeated blending operations may be used to distribute the active agent throughout those compositions employing large quantities of fillers. Such operations are conventional in the art. The tablets may for example be prepared by wet or dry granulation and optionally coated according to methods well known in normal pharmaceutical practice, in particular with an enteric coating.

The pharmaceutical compositions may also be adapted for parenteral administration, such as sterile solutions, suspensions or lyophilized products in the appropriate unit dosage form. Adequate excipients can be used, such as bulking agents, buffering agents or surfactants.

The mentioned formulations will be prepared using standard methods such as those described or referred to in the Spanish and US Pharmacopoeias and similar reference texts.

Administration of the compounds or compositions of the present invention may be by any suitable method, such as intravenous infusion, oral preparations, and intraperitoneal and intravenous administration. Oral administration is preferred because of the convenience for the patient and the chronic character of the diseases to be treated.

Generally an effective administered amount of a compound of the invention will depend on the relative efficacy of the compound chosen, the severity of the disorder being treated and the weight of the sufferer. However, active compounds will typically be administered once or more times a day for example 1, 2, 3 or 4 times daily, with typical total daily doses in the range of from 0.1 to 1000 mg/kg/day.

The compounds and compositions of this invention may be used with other drugs to provide a combination therapy. The other drugs may form part of the same composition, or be provided as a separate composition for administration at the same time or at different time.

Another aspect of the invention refers to the use of a compound of the invention or a pharmaceutically acceptable salt or isomer thereof in the manufacture of a medicament.

Another aspect of the invention refers to a compound of the invention according as described above according to general formula I, or a pharmaceutically acceptable salt or isomer thereof, for use as a medicament for the treatment of pain. Preferably the pain is medium to severe pain, visceral pain, chronic pain, cancer pain, migraine, inflammatory pain, acute pain or neuropathic pain, allodynia or hyperalgesia. This may include mechanical allodynia or thermal hyperalgesia.

Another aspect of the invention refers to the use of a compound of the invention in the manufacture of a medicament for the treatment or prophylaxis of pain.

In a preferred embodiment the pain is selected from medium to severe pain, visceral pain, chronic pain, cancer pain, migraine, inflammatory pain, acute pain or neuropathic pain, allodynia or hyperalgesia, also preferably including mechanical allodynia or thermal hyperalgesia.

Another aspect of this invention relates to a method of treating or preventing pain which method comprises administering to a patient in need of such a treatment a therapeutically effective amount of a compound as above defined or a pharmaceutical composition thereof. Among the pain syndromes that can be treated are medium to severe pain, visceral pain, chronic pain, cancer pain, migraine, inflammatory pain, acute pain or neuropathic pain, allodynia or hyperalgesia, whereas this could also include mechanical allodynia or thermal hyperalgesia.

The present invention is illustrated below with the aid of examples. These illustrations are given solely by way of example and do not limit the general spirit of the present invention.

### General Experimental Part (Methods and Equipment of the Synthesis and Analysis

### Synthesis Description

A two-step process is described for the preparation of compounds of general formula (I) starting from a compound of formula II, as shown in Scheme 1.

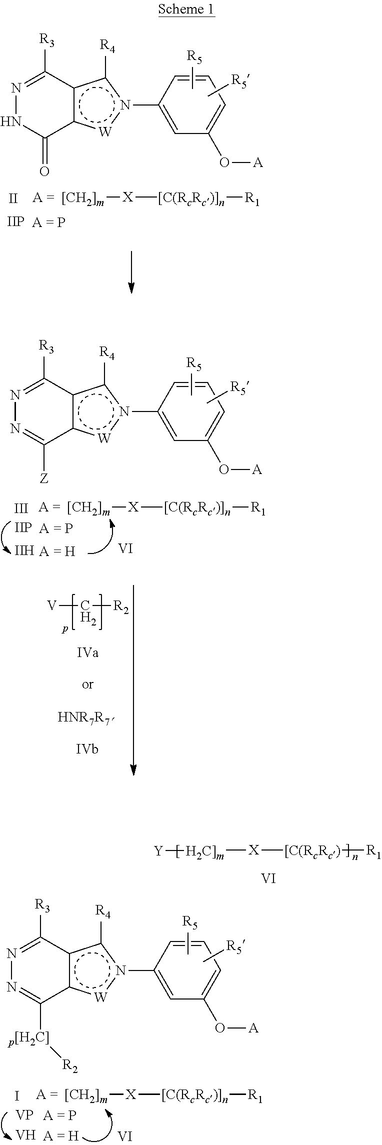

wherein R₁, R₂, R₃, R₄, R₅, R_{5′}, R₇, R_{7′}, R_{c}, R_{c′}, W, X, m, n and p have the meanings as defined above for a compound of formula (I), Y represents a leaving group (such as halogen, mesylate, tosylate, nosylate or triflate) or OH, Z represents an halogen (preferably chloro) or triflate, V represents a suitable organometallic reagent (preferably a boron or zinc reagent) and P represents a suitable protecting group (such as alkyl or benzyl, preferably methyl).

The two-step process can be carried out as described below:

Step 1: A compound of formula III, where Z represents chloro, can be prepared by treating a compound of formula II with a suitable chlorinating reagent such as phosphorus oxychloride, optionally in the presence of a suitable solvent, preferably heating. When Z represents a triflate group, the reaction can be performed by treating a compound of formula II with trifluoromethane sulphonic anhydride in the presence of pyridine.
Step 2: A compound of formula I can be prepared by reacting a compound of formula III with suitable compounds and reaction conditions depending on the meaning of the group [CH₂]ₚR₂:
a) When the group [CH₂]ₚR₂ is attached through a carbon atom, the reaction can be performed by treating a compound of formula III with a suitable organometallic reagent of formula IVa, preferably a boron or zinc reagent. The coupling reaction may be carried out using a suitable catalyst (preferably a Pd catalyst) and a suitable ligand (preferably a phosphine ligand), in the presence of a base, such as K₂CO₃ or Cs₂CO₃, in a suitable polar solvent, such as dioxane.
b) When the group [CH₂]ₚR₂ is attached through a nitrogen atom, as is the case when p is 0 and R₂ is NR₇R_{7′}, the reaction may be performed by treating a compound of formula III with an amine of formula IVb in a suitable solvent, such as isopropanol, ethanol or acetonitrile; optionally in the presence of an organic base such as triethylamine or diisopropylethylamine or an inorganic base such as K₂CO₃ or Cs₂CO₃; at a suitable temperature comprised between room temperature and the reflux temperature, preferably heating, or alternatively, it can be carried out in a microwave reactor. Alternatively, the amine of formula IVb can be introduced, using a Pd catalysed procedure in the presence of a suitable catalyst, a suitable ligand (preferably a phosphine ligand) a suitable base and a suitable solvent, such as dioxane.

The group [CH₂]ₘ—X—[C(R_{c}R_{c′})]ₙ—R₁ can be present from the beginning of the synthesis or alternatively it can be incorporated later on, by reaction of a compound of formula IIIH or VH with a compound of formula VI to render a compound of formula III or I, respectively. A compound of formula IIIH or VH is obtained, in turn, by deprotection of a compound of formula IIIP or VP, wherein P represents a suitable protecting group, such as alkyl or benzyl. When the protecting group P represents alkyl (preferably methyl), the deprotection is carried out by treating a compound of formula IIIP or VP with boron tribromide or boron trichloride, in a suitable solvent such as dichloromethane, at a suitable temperature, preferably cooling below 0° C. When the protecting group P represents benzyl, the deprotection reaction is preferably carried out by hydrogenation under hydrogen atmosphere and metal catalysis, preferably by the use of palladium over charcoal as catalyst in a suitable solvent such as methanol or ethanol, optionally in the presence of an acid such as acetic or hydrochloric acid.

The reaction of a phenol of formula IIIH or VH with a compound of formula VI to render a compound of formula III or I, respectively, can be carried out under different reaction conditions depending on the meaning of Y:
a) When Y represents a leaving group such as halogen, mesylate, tosylate, nosylate or triflate, the reaction is carried out in a suitable polar solvent, such as dimethylformamide; in the presence of a base such as K₂CO₃, Cs₂CO₃, sodium hydride or potassium tert-butoxide; at a suitable temperature comprised between room temperature and the reflux temperature, preferably heating, or alternatively, the reaction can be carried out in a microwave reactor. Additionally, an activating agent such as sodium iodide can be used.
b) When Y represents OH, the reaction can be carried out by treating a phenol of formula IIIH or VH with an alcohol of formula VI in the presence of an azo compound such as 1,1′-(azodicarbonyl)dipiperidine (ADDP), diisopropylazodicarboxylate (DIAD) or diethyl azodicarboxylate (DEAD) and a phosphine such as tributylphosphine or triphenylphoshine. The reaction is carried out in a suitable solvent, such as toluene or tetrahydrofuran; at a suitable temperature comprised between room temperature and the reflux temperature, preferably heating, or alternatively, the reactions can be carried out in a microwave reactor.

Alternatively, a compound of formula VH can be synthesized from a compound of formula IIIH by reaction with a compound of formula IVa or IVb, following the conditions described for the preparation of a compound of formula I from a compound of formula III.

The compounds of formula IIIP and VP can be obtained following the two-step process described in Scheme 1, starting from a protected compound of formula IIP.

The compounds of formula II and IIP can be synthesized following procedures described in the literature. As a way of example, the preparation of compounds of general formula II or IIP, wherein W represents nitrogen (compounds of formula IIa or IIaP), is described in Scheme 2:

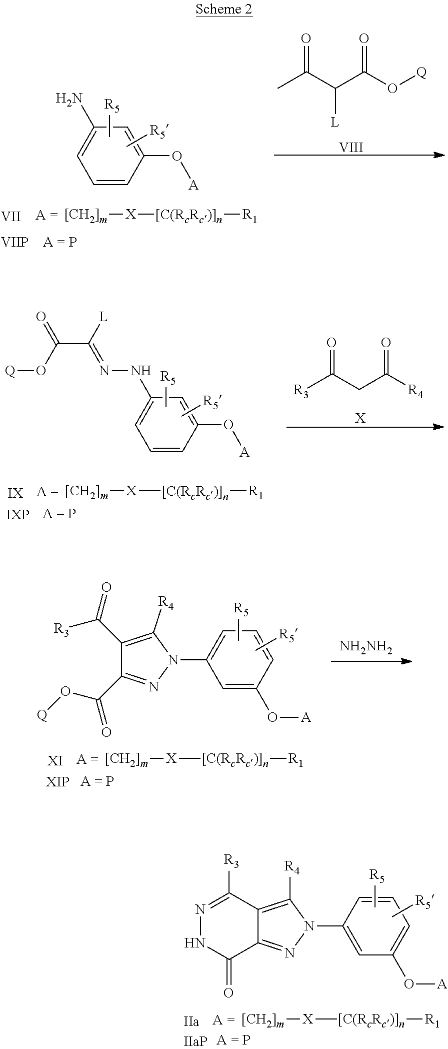

wherein R₁, R₂, R₃, R₄, R₅, R_{5′}, R_{c}, R_{c′}, X, m, n and p have the meanings as defined above for a compound of formula (I), L represents halogen, Q represents an alkyl group (preferably methyl or ethyl) and P represents a suitable protecting group (such as alkyl or benzyl, preferably methyl).

The reaction of an aniline compound of formula VII with a compound of formula VIII under suitable reaction conditions renders a compound of formula IX. As a way of example, when L represents Cl, a compound of formula IX can be prepared by treating a compound of formula VII with sodium nitrite in a mixture of hydrochloric acid and ethanol at 0° C., followed by reaction with a compound of formula VIII in a mixture of ethanol and water at room temperature.

A compound of formula IX is then reacted with a di-keto compound of formula X to give a pyrazole of formula XI. The reaction is carried out in the presence of a strong base such as sodium ethoxide and in a suitable solvent such as ethanol.

Finally, treatment of a compound of formula XI with hydrazine, in a suitable solvent such as ethanol or acetic acid, at a suitable temperature, preferably heating, yields a compound of formula IIa.

Following the same sequence described in Scheme 2 starting from a conveniently protected aniline of formula VIIP, the corresponding protected compound of formula IIaP can be obtained.

The preparation of compounds of general formula I wherein W represents CR_{4′} and wherein the group [CH₂]ₚR₂ is the same as R₃ (compounds of formula Ib) may be carried out as described in Scheme 3

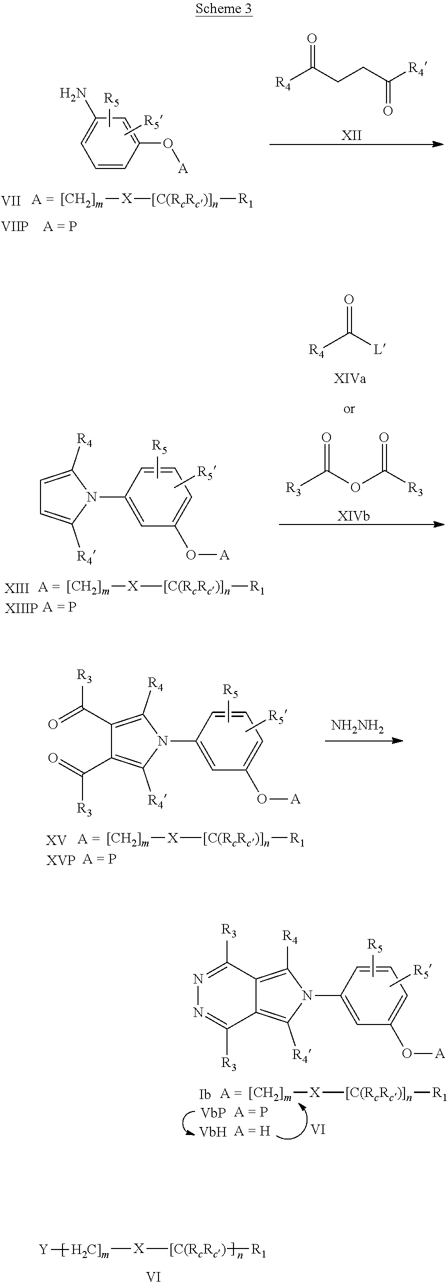

wherein R₁, R₃, R₄, R_{4′}, R₅, R_{5′}, R_{c}, R_{c′}, X, m and n have the meanings as defined above for a compound of formula (I), L′ represents halogen, Y represents a leaving group (such as halogen, mesylate, tosylate, nosylate or triflate) or OH and P represents a suitable protecting group (such as alkyl or benzyl, preferably methyl).

The reaction of an aniline compound of formula VII with a di-keto compound of formula XII, in a suitable solvent such as toluene and optionally in the presence of an acid such as acetic acid, renders a pyrrole compound of formula XIII.

A compound of formula XIII is then treated with an acylating agent of formula XIVa or XIVb to give an acylpyrrole of formula XV. The reaction is carried out using a Lewis acid such as tin(IV) chloride or aluminum chloride, in a suitable solvent such as dichloromethane, dichloroethane or toluene, or mixtures thereof.

Finally, treatment of a compound of formula XV with hydrazine, in a suitable solvent such as ethanol or acetic acid or mixtures thereof, at a suitable temperature comprised between room temperature and the reflux temperature, preferably at room temperature, yields a compound of formula Ib.

Following the same sequence described in Scheme 3 starting from a conveniently protected aniline of formula VIIP, the corresponding precursor compound of formula VbP can be obtained.

The compounds of general formula IVa, IVb, VI, VII, VIIP, VIII, X, XII, XIVa and XIVb wherein R₁, R₂, R₇, R_{7′}, R_{c}, R_{c′}, R₆, R_{6′}, L, P, Q, V, Y, X, m, n and p have the meanings as defined above, are commercially available or can be prepared by conventional methods described in the bibliography.

Moreover, certain compounds of the present invention can also be obtained starting from other compounds of formula (I) by appropriate conversion reactions of functional groups, in one or several steps, using well-known reactions in organic chemistry under standard experimental conditions. As a way of example, some of these conversions include the reductive amination of an amino group with an aldehyde or ketone to prepare a further substituted amino group; or the hydrolysis of a cyano group to yield the corresponding carboxamido group.

In some of the processes described above it may be necessary to protect the reactive or labile groups present with suitable protecting groups, such as for example Boc (tert-butoxycarbonyl) for the protection of amino groups. The procedures for the introduction and removal of these protecting groups are well known in the art and can be found thoroughly described in the literature.

In addition, a compound of formula I that shows chirality can also be obtained by resolution of a racemic compound of formula I either by chiral preparative HPLC or by crystallization of a diastereomeric salt or co-crystal. Alternatively, the resolution step can be carried out at a previous stage, using any suitable intermediate.

### EXAMPLES

The following abbreviations are used in the examples:

ACN: acetonitrile
AcOH: acetic acid
Anh: anhydrous
ADDP: 1,1′-(azodicarbonyl)dipiperidine
Boc: tert-butoxycarbonyl
Conc: concentrated
DCM: dichloromethane
DEA: diethylamine
DMF: dimethylformamide
Eq: equivalent/s
EtOAc; ethyl acetate
EtOH: ethanol
EX: example
h: hour/s
HPLC: high performance liquid chromatography
INT: intermediate

### IPA: Isopropanol

MeOH: methanol
MS: mass spectrometry
Min.: minutes
Quant: quantitative
Ret.: retention
r.t.: room temperature
Sat: saturated
s.m.: starting material
TEA: triethylamine
TFA: trifluoroacetic acid
THF: tetrahydrofuran
Wt: weight

The following methods were used to determine the HPLC-MS spectra:

### Method A

### Column: Gemini-NX 30×4.6 mm, 3 um

### Temperature: 40° C.

Flow: 2.0 mL/min
Gradient: NH₄HCO₃ pH 8: ACN (95:5) - - - 0.5 min - - - (95:5) - - - 6.5 min - - - (0:100) - - - 1 min - - - (0:100)
Sample dissolved approx. 1 mg/mL in NH₄HCO₃ pH 8/ACN

### Method B

### Column: Kinetex EVO 50×4.6 mm, 2.6 um

### Temperature: 40° C.

Flow: 2.0 mL/min
Gradient: NH₄HCO₃ pH 8: ACN (95:5) - - - 0.5 min - - - (95:5) - - - 6.5 min - - - (0:100) - - - 1 min - - - (0:100)
Sample dissolved approx. 1 mg/mL in NH₄HCO₃ pH 8/ACN

### Method C

### Column: Kinetex EVO 50×4.6 mm, 2.6 um

### Temperature: 40° C.

Flow: 1.5 mL/min
Gradient: NH₄HCO₃ pH 8: ACN (95:5) - - - 0.5 min - - - (95:5) - - - 6.5 min - - - (0:100) - - - 2 min - - - (0:100)
Sample dissolved approx. 1 mg/mL in NH₄HCO₃ pH 8/ACN

### Synthesis of Intermediates

### Intermediate 1: 7-Chloro-2-(3-methoxyphenyl)-3,4-dimethyl-2H-pyrazolo[3,4-d]pyridazine

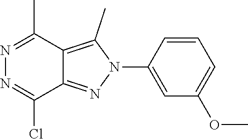 [SMILES: COc1cccc(-n2nc3c(Cl)nnc(C)c3c2C)c1]

Step 1. (Z)-Ethyl 2-chloro-2-(2-(3-methoxyphenyl)hydrazono)acetate: To a cooled solution of 3-methoxyaniline (20 g, 162 mmol) in a mixture of conc. HCl (32 mL) and EtOH (32 mL), a solution of sodium nitrite (11.2 g, 162 mmol) in water (20 mL) was added dropwise. After stirring for 20 min at 0° C., ethyl 2-chloro-3-oxobutanoate (22.5 mL, 162 mmol) was added. The mixture was diluted with EtOH/water 9:1 v/v (360 mL). Finally, sodium acetate (13.3 g, 162 mmol) was added and it was stirred at room temperature for 2 h. Water (800 mL) was then added, the resulting suspension was filtered and the collected solids were dried under vacuum to afford the title compound (33.7 g, 81% yield).

Step 2. Ethyl 4-acetyl-1-(3-methoxyphenyl)-5-methyl-1H-pyrazole-3-carboxylate: Acetylacetone (12 mL, 117 mmol) was added to sodium ethoxide (21 wt % in ethanol, 43.6 mL, 117 mmol) and the mixture was stirred at r.t. overnight. Then, the product obtained in step 1 (30 g, 117 mmol) was added. The mixture was stirred at r.t. for 4 h and then it was left standing for 18 h without stirring. Water (420 mL) was added, the suspension was filtered and the solids were dried under vacuum to afford the title compound (28.5 g, 81% yield).

Step 3. 2-(3-Methoxyphenyl)-3,4-dimethyl-2H-pyrazolo[3,4-d]pyridazin-7(6H)-one: To a solution of the product obtained in step 2 (28.5 g, 94.3 mmol) in EtOH (200 mL), hydrazine (50-60% in water, 27 mL, 282 mmol) was added and the mixture was heated to reflux for 5 h. The suspension was cooled to room temperature and the solids were filtered, washed with cold EtOH and dried under vacuum to obtain afford the title compound (22.4 g, 88% yield).

Step 4. Title compound: A mixture of the product obtained in step 3 (22.4 g, 82.9 mmol) and POCl₃ (112 mL) was heated at 100° C. for 3 h. POCl₃ was then distilled off. The residue was cooled to 0° C. and it was basified to pH 8 by careful addition (exothermic reaction) of ice and 24% NaOH (approx. 110 mL). The precipitated solids were stirred for 1-2 h at r.t. in order to obtain a filterable suspension and then they were collected by filtration, washed with water and dried under vacuum to yield the title compound (23.0 g, 96% yield).

### Intermediate 2: 3-(7-Chloro-3,4-dimethyl-2H-pyrazolo[3,4-d]pyridazin-2-yl)phenol

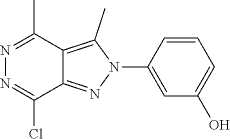 [SMILES: Cc1nnc(Cl)c2nn(-c3cccc(O)c3)c(C)c12]

To a solution of intermediate 1 (23 g, 80 mmol) in DCM (345 mL), cooled at −78° C., BBr₃ (1M solution in DCM, 278 mL, 80 mmol) was added dropwise. The reaction was slowly warmed to −50° C. during 2 h. Then 1M NaOH was added to adjust pH to 8-9. DCM was distilled off and the resultant suspension was filtered. The solids were filtered, washed with water and dried under vacuum to obtain the title compound (30.3 g, overweight, quantitative yield assumed) as a crude product that was used as such without further purification.

### Intermediate 3: 3-(7-(Dimethylamino)-3,4-dimethyl-2H-pyrazolo[3,4-d]pyridazin-2-yl)phenol

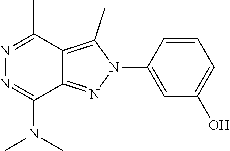 [SMILES: Cc1nnc(N(C)C)c2nn(-c3cccc(O)c3)c(C)c12]

In a round-bottom pressure flask, a mixture of intermediate 2 (63 wt %, 31 g, 72.7 mmol), TEA (20.2 mL, 145 mmol) and dimethylamine (33% in EtOH, 32.7 mL, 182 mmol) in IPA (320 mL) was heated at 120° C. overnight. Then, it was concentrated to dryness. The residue was slurried in water, filtered, washed with water and dried under vacuum to render the title compound (17 g, 82% yield).

Intermediate 3 has been alternatively prepared following the procedure described for the preparation of intermediate 2, using intermediate 4 as starting material.

This method was used for the preparation of Intermediates 4-6 using suitable starting materials:

| INT | Structure | Chemical name | s.m. |
|---|---|---|---|
| 4 | 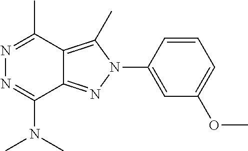 [SMILES: COc1cccc(-n2nc3c(N(C)C)nnc(C)c3c2C)c1] | 2-(3-methoxyphenyl)- N,N,3,4-tetramethyl-2H- pyrazolo[3,4-d] pyridazin-7-amine | Interm. 1 |
| 5 | 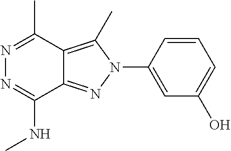 [SMILES: CNc1nnc(C)c2c(C)n(-c3cccc(O)c3)nc12] | 3-(3,4-dimethyl-7- (methylamino)-2H- pyrazolo[3,4-d] pyridazin-2- | Interm. 2 |
| 6 | 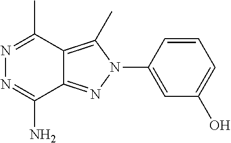 [SMILES: Cc1nnc(N)c2nn(-c3cccc(O)c3)c(C)c12] | 3-(7-amino-3,4- dimethyl-2H- pyrazolo[3,4-d] pyridazin-2- | Interm. 2 |

### Intermediate 7: 3-(1,4,5,7-Tetramethyl-6H-pyrrolo[3,4-d]pyridazin-6-yl)phenol

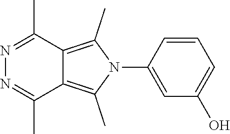 [SMILES: Cc1nnc(C)c2c(C)n(-c3cccc(O)c3)c(C)c12]

Step 1. 1-(3-Methoxyphenyl)-2,5-dimethyl-1H-pyrrole: A mixture of 3-methoxyaniline (10.79 g, 88 mmol), hexane-2,5-dione (10.3 mL, 88 mmol) and AcOH (0.9 mL) in toluene (640 mL) was heated to reflux for 3 days. The solvent was evaporated to dryness and the residue was dried under vacuum to afford the title compound as a crude product (19.6 g, overweight, 17.63 g theoretical weight, quant yield assumed).

Step 2. 1,1′-(1-(3-Methoxyphenyl)-2,5-dimethyl-1H-pyrrole-3,4-diyl)diethanone: To a solution of the product obtained in Step 1 (19.6 g, 17.63 g theoretical weight, 88 mmol) in toluene (265 mL), cooled to 0-5° C., SnCl₄ solution (1 M in DCM, 88 mL, 88 mmol) was added dropwise followed by acetyl chloride (12.5 mL, 175 mmol). The reaction mixture was heated at 50° C. overnight. 6 N NaOH was added and it was extracted with EtOAc. The combined organic phases were washed with brine, dried over Na₂SO₄ and concentrated to dryness to afford the title compound (27.1 g, overweight, 24.9 g theoretical weight, quant yield assumed).

Step 3. 6-(3-Methoxyphenyl)-1,4,5,7-tetramethyl-6H-pyrrolo[3,4-d]pyridazine: To a solution of the product obtained in Step 2 (27.1 g, 24.9 g theoretical weight, 88 mmol) in EtOH (500 mL), hydrazine mono-hydrate (12 mL, 123 mmol) and a few drops of acetic acid were added and the mixture was stirred at r.t. overnight. Then, it was poured onto crushed ice, EtOH was distilled off and the aqueous phase was extracted several times with DCM. The combined organic phases were washed with brine, dried over Na₂SO₄ and concentrated to dryness. The crude product was purified by flash chromatography, silica gel, gradient DCM to MeOH:DCM (1:4) to give the title compound (15.0 g, 61% yield).

Step 4. 3-(1,4,5,7-Tetramethyl-6H-pyrrolo[3,4-d]pyridazin-6-yl)phenol: BBr₃ (1 M solution in DCM, 35.5 mL, 35.5 mmol) was added dropwise to a solution of the product obtained in Step 3 (2.0 g, 7.11 mmol) in DCM (15 mL), cooled at −78° C., and the mixture was stirred at −30° C. for 3 h. Ice was added to quench the reaction and then DCM was distilled off. 1 N NaOH was added to adjust the pH to 9 and the resulting suspension was stirred at r.t. overnight. The solids were then filtered, washed with water and dried under vacuum to obtain the title compound (2.88 g, overweight, 1.9 g theoretical weight, quant yield assumed) as a crude product that was used as such without further purification.

### Synthesis of Examples

### Example 1: N,N,3,4-Tetramethyl-2-(3-(3-(methylamino)-1-phenylpropoxy)phenyl)-2H-pyrazolo[3,4-d]pyridazin-7-amine

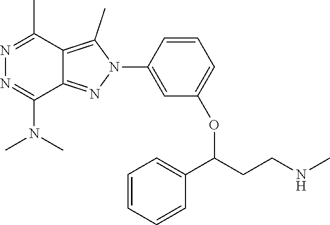 [SMILES: CNCCC(Oc1cccc(-n2nc3c(N(C)C)nnc(C)c3c2C)c1)c1ccccc1]

Step 1. tert-Butyl (3-chloro-3-phenylpropyl)(methyl)carbamate: To a cooled solution of 3-(methylamino)-1-phenylpropan-1-ol (10 g, 60.5 mmol) in DCM (40 mL), a solution of SOCl₂ (5.3 mL, 72.6 mmol) in DCM (20 mL) was added dropwise. The mixture was stirred at r.t. for 2 h and then the solvent was concentrated to dryness. The crude product thus obtained was dissolved in tert-butanol (52 mL). 1.8 M NaOH solution (70 mL, 127 mmol) and di-tert-butyl dicarbonate (14.5 g, 66.6 mmol) were added and the reaction mixture was stirred for 15 min. Brine and DCM were added, the phases were separated and the aqueous layer was extracted with DCM. The combined organic phases were washed with brine, dried with Na₂SO₄ and concentrated to dryness to obtain the title compound as a crude product (16.3 g, 95% yield), that was used without further purification.

Step 2. tert-Butyl (3-(3-(7-(dimethylamino)-3,4-dimethyl-2H-pyrazolo[3,4-d]pyridazin-2-yl)phenoxy)-3-phenylpropyl)(methyl)carbamate. A suspension of intermediate 3 (0.6 g, 1.88 mmol), K₂CO₃ (0.78 g, 5.6 mmol) and the crude product obtained in step 1 (0.64 g, 2.25 mmol) in DMF (12 mL) was heated in a sealed tube at 80° C. overnight. Water and EtOAc were added to the cooled reaction mixture and the phases were separated. The aqueous phase was extracted twice with EtOAc. The combined organic phases were washed with brine, dried with Na₂SO₄ and concentrated to dryness. The residue was purified by flash chromatography, silica gel, gradient DCM to MeOH:DCM (1:4) to give the title compound (658 mg, 66% yield).

Step 3. Title compound: To a solution of the product obtained in step 2 (658 mg, 1.24 mmol) in DCM (16 mL), TFA (1 mL, 12.4 mmol) was added and the reaction mixture was stirred at r.t. until full conversion. It was concentrated to dryness and the residue was re-dissolved in DCM and washed with 1M NaOH. The organic phase was dried with Na₂SO₄ and concentrated. The crude product was purified by flash chromatography, silica gel, gradient DCM to MeOH:DCM (1:4) to give the title compound (305 mg, 57% yield).

HPLC retention time (method A): 3.26 min; MS: 431.2 (M+H).

This method was used for the preparation of Examples 2-29 using suitable starting materials:

| | | | Ret | | |
|---|---|---|---|---|---|
| | | | time | MS | HPLC |
| EX | Structure | Chemical name | (min) | (M + H) | Method |
| 2 | 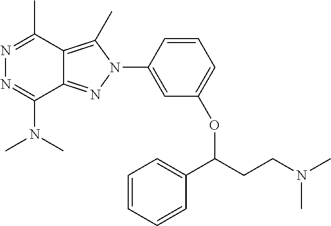 [SMILES: Cc1nnc(N(C)C)c2nn(-c3cccc(OC(CCN(C)C)c4ccccc4)c3)c(C)c12] | 2-(3-(3- (dimethylamino)-1- phenylpropoxy)phenyl- N,N,3,4-tetramethyl- 2H-pyrazolo[3,4-d] pyridazin-7-amine | 4.00 | 445.2 | A |
| 3 | 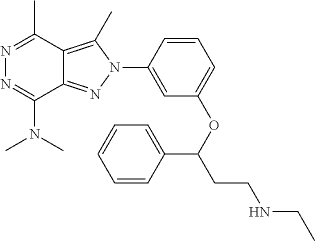 [SMILES: CCNCCC(Oc1cccc(-n2nc3c(N(C)C)nnc(C)c3c2C)c1)c1ccccc1] | 2-(3-(3-(ethylamino)-1- phenylpropoxy)phenyl)- N,N,3,4-tetramethyl- 2H-pyrazolo[3,4-d] pyridazin-7-amine | 3.66 | 445.2 | A |
| 4 | 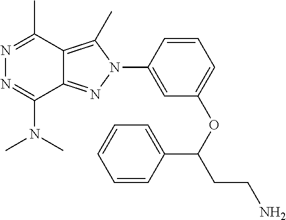 [SMILES: Cc1nnc(N(C)C)c2nn(-c3cccc(OC(CCN)c4ccccc4)c3)c(C)c12] | 2-(3-(3-amino-1- phenylpropoxy)phenyl- N,N,3,4-tetramethyl- 2H-pyrazolo[3,4-d] pyridazin-7-amine | 3.37 | 417.2 | A |
| 5 | 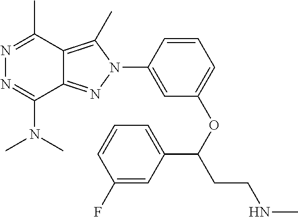 [SMILES: CNCCC(Oc1cccc(-n2nc3c(N(C)C)nnc(C)c3c2C)c1)c1cccc(F)c1] | 2-(3-(1-(3- fluorophenyl)-3- (methylamino)propoxy) phenyl)-N,N,3,4- tetramethyl-2H- pyrazolo[3,4-d] pyridazin-7-amine | 3.79 | 449.1 | A |
| 6 | 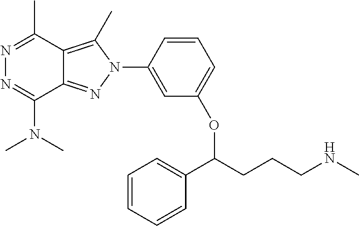 [SMILES: CNCCCC(Oc1cccc(-n2nc3c(N(C)C)nnc(C)c3c2C)c1)c1ccccc1] | N,N,3,4-tetramethyl-2- (3-(4-(methylamino)-1- phenylbutoxy)phenyl)- 2H-pyrazolo[3,4-d] pyridazin-7-amine | 3.71 | 445.2 | A |
| 7 | 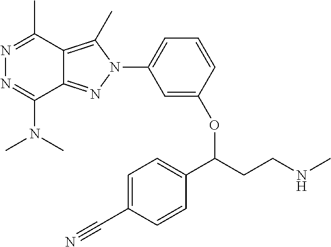 [SMILES: CNCCC(Oc1cccc(-n2nc3c(N(C)C)nnc(C)c3c2C)c1)c1ccc(C#N)cc1] | 4-(1-(3-(7- (dimethylamino)-3,4- dimethyl-2H- pyrazolo[3,4-d] pyridazin-2- yl)phenoxy)-3- (methylamino)propyl) benzonitrile | 3.84 | 456.2 | A |
| 8 | 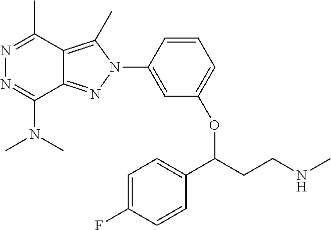 [SMILES: CNCCC(Oc1cccc(-n2nc3c(N(C)C)nnc(C)c3c2C)c1)c1ccc(F)cc1] | 2-(3-(1-(4- fluorophenyl)-3- (methylamino)propoxy) phenyl)-N,N,3,4- tetramethyl-2H- pyrazolo[3,4-d] pyridazin-7-amine | 3.97 | 449.2 | A |
| 9 | 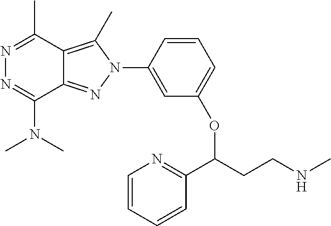 [SMILES: CNCCC(Oc1cccc(-n2nc3c(N(C)C)nnc(C)c3c2C)c1)c1ccccn1] | N,N,3,4-tetramethyl-2- (3-(3-(methylamino)-1- (pyridin-2- yl)propoxy)phenyl)-2H- pyrazolo[3,4-d] pyridazin-7-amine | 3.38 | 432.1 | A |
| 10 | 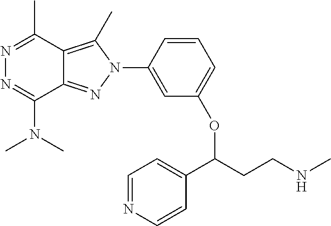 [SMILES: CNCCC(Oc1cccc(-n2nc3c(N(C)C)nnc(C)c3c2C)c1)c1ccncc1] | N,N,3,4-tetramethyl-2- (3-(3-(methylamino)-1- (pyridin-4- yl)propoxy)phenyl)-2H- pyrazolo[3,4-d] pyridazin-7-amine | 3.38 | 432.1 | A |
| 11 | 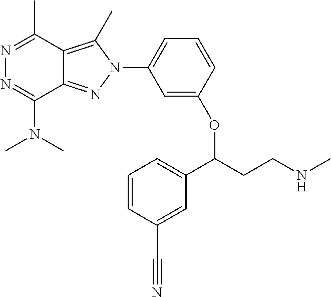 [SMILES: CNCCC(Oc1cccc(-n2nc3c(N(C)C)nnc(C)c3c2C)c1)c1cccc(C#N)c1] | 3-(1-(3-(7- (dimethylamino)-3,4- dimethyl-2H- pyrazolo[3,4-d] pyridazin-2- yl)phenoxy)-3- (methylamino)propyl) bezonitrile | 3.96 | 456.1 | A |
| 12 | 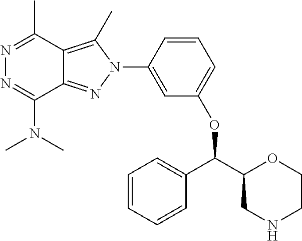 [SMILES: Cc1nnc(N(C)C)c2nn(-c3cccc(O[C@H](c4ccccc4)[C@@H]4CNCCO4)c3)c(C)c12] | N,N,3,4-tetramethyl-2- (3-((R)-((S)-morpholin- 2-yl)(phenyl) methoxy) phenyl)-2H-pyrazolo [3,4-d] pyridazin-7-amine ⁽¹⁾ | 3.69 | 459.1 | A |
| 13 | 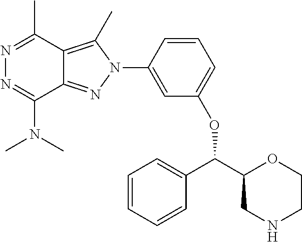 [SMILES: Cc1nnc(N(C)C)c2nn(-c3cccc(O[C@@H](c4ccccc4)[C@@H]4CNCCO4)c3)c(C)c12] | N,N,3,4-tetramethyl-2- (3-((S)-((S)-morpholin- 2-yl)(phenyl)methoxy) phenyl)-2H-pyrazolo [3,4-d] pyridazin-7-amine ⁽²⁾ | 3.53 | 459.2 | A |
| 14 | 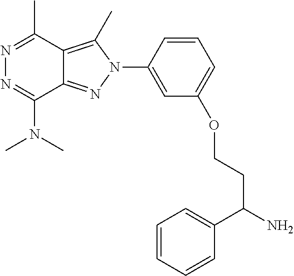 [SMILES: Cc1nnc(N(C)C)c2nn(-c3cccc(OCCC(N)c4ccccc4)c3)c(C)c12] | 2-(3-(3-amino-3- phenylpropoxy)phenyl)- N,N,3,4-tetramethyl- 2H-pyrazolo[3,4-d] pyridazin-7-amine | 3.76 | 417.2 | A |
| 15 | 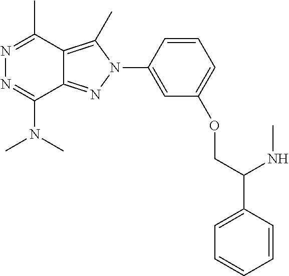 [SMILES: CNC(COc1cccc(-n2nc3c(N(C)C)nnc(C)c3c2C)c1)c1ccccc1] | N,N,3,4-tetramethyl-2- (3-(2-(methylamino)-2- phenylethoxy)phenyl)- 2H-pyrazolo[3,4-d] pyridazin-7-amine | 3.99 | 417.2 | A |
| 16 | 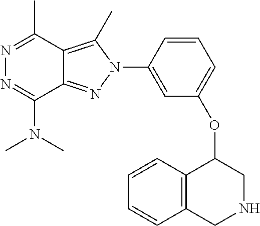 [SMILES: Cc1nnc(N(C)C)c2nn(-c3cccc(OC4CNCc5ccccc54)c3)c(C)c12] | N,N,3,4-tetramethyl-2- (3-((1,2,3,4- tetrahydroisoquinolin-4- yl)oxy)phenyl)-2H- pyrazolo[3,4-d] pyridazin-7-amine | 3.67 | 415.2 | A |
| 17 | 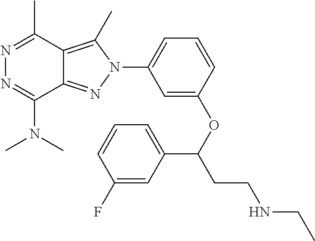 [SMILES: CCNCCC(Oc1cccc(-n2nc3c(N(C)C)nnc(C)c3c2C)c1)c1cccc(F)c1] | 2-(3-(3-(ethylamino)-1- (3-fluorophenyl) propoxy) phenyl)-N,N,3,4- tetramethyl-2H- pyrazolo[3,4-d] pyridazin-7-amine | 3.75 | 463.3 | B |
| 18 | 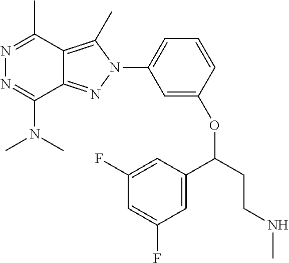 [SMILES: CNCCC(Oc1cccc(-n2nc3c(N(C)C)nnc(C)c3c2C)c1)c1cc(F)cc(F)c1] | 2-(3-(1-(3,5- difluorophenyl)-3- (methylamino)propoxy) phenyl)-N,N,3,4- tetramethyl-2H- pyrazolo[3,4-d] pyridazin-7-amine | 3.74 | 467.2 | B |
| 19 | 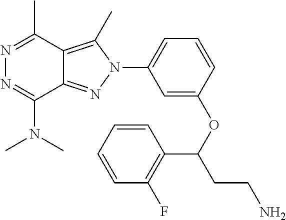 [SMILES: Cc1nnc(N(C)C)c2nn(-c3cccc(OC(CCN)c4ccccc4F)c3)c(C)c12] | 2-(3-(3-amino-1-(2- fluorophenyl)propoxy) phenyl)-N,N,3,4- tetramethyl-2H- pyrazolo[3,4-d] pyridazin-7-amine | 3.4 | 435.2 | B |
| 20 | 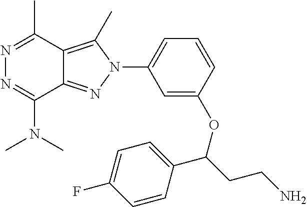 [SMILES: Cc1nnc(N(C)C)c2nn(-c3cccc(OC(CCN)c4ccc(F)cc4)c3)c(C)c12] | 2-(3-(3-amino-1-(4- fluorophenyl)propoxy) phenyl)-N,N,3,4- tetramethyl-2H- pyrazolo[3,4-d] pyridazin-7-amine | 3.49 | 435.2 | B |
| 21 | 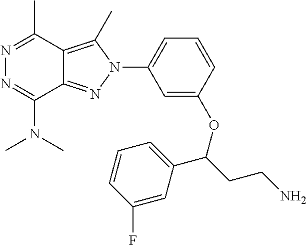 [SMILES: Cc1nnc(N(C)C)c2nn(-c3cccc(OC(CCN)c4cccc(F)c4)c3)c(C)c12] | 2-(3-(3-amino-1-(3- fluorophenyl)propoxy) phenyl)-N,N,3,4- tetramethyl-2H- pyrazolo[3,4-d] pyridazin-7-amine | 3.48 | 435.2 | B |
| 22 | 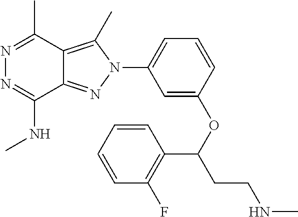 [SMILES: CNCCC(Oc1cccc(-n2nc3c(NC)nnc(C)c3c2C)c1)c1ccccc1F] | 2-(3-(1-(2- fluorophenyl)-3- (methylamino)propoxy) phenyl)-N,3,4-trimethyl- 2H-pyrazolo[3,4-d] pyridazin-7-amine | 3.17 | 435.2 | C |
| 23 | 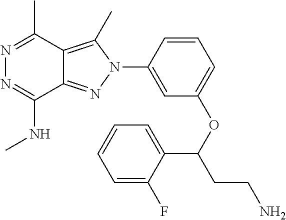 [SMILES: CNc1nnc(C)c2c(C)n(-c3cccc(OC(CCN)c4ccccc4F)c3)nc12] | 2-(3-(3-amino-1-(2- fluorophenyl)propoxy) phenyl)-N,3,4-trimethyl- 2H-pyrazolo[3,4-d] pyridazin-7-amine | 3.44 | 421.0 | C |
| 24 | 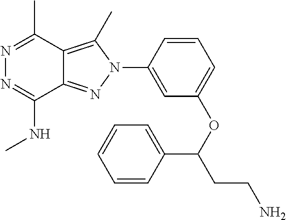 [SMILES: CNc1nnc(C)c2c(C)n(-c3cccc(OC(CCN)c4ccccc4)c3)nc12] | 2-(3-(3-amino-1- phenylpropoxy)phenyl)- N,3,4-trimethyl-2H- pyrazolo[3,4-d] pyridazin-7-amine | 3.42 | 403.1 | C |
| 25 | 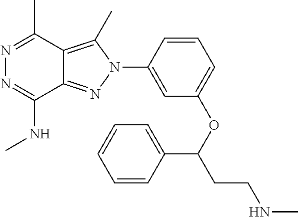 [SMILES: CNCCC(Oc1cccc(-n2nc3c(NC)nnc(C)c3c2C)c1)c1ccccc1] | N,3,4-trimethyl-2-(3-(3- (methylamino)-1- phenylpropoxy)phenyl)- 2H-pyrazolo[3,4-d] pyridazin-7-amine | 3.46 | 417.1 | C |
| 26 | 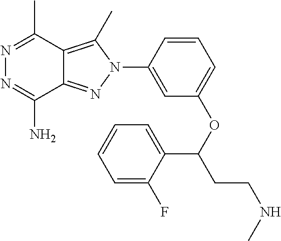 [SMILES: CNCCC(Oc1cccc(-n2nc3c(N)nnc(C)c3c2C)c1)c1ccccc1F] | 2-(3-(1-(2- fluorophenyl)-3- (methylamino)propoxy) phenyl)-3,4-dimethyl- 2H-pyrazolo[3,4-d] pyridazin-7-amine | 3.41 | 421.0 | C |
| 27 | 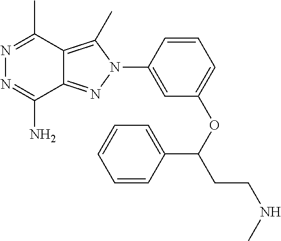 [SMILES: CNCCC(Oc1cccc(-n2nc3c(N)nnc(C)c3c2C)c1)c1ccccc1] | 3,4-dimethyl-2-(3-(3- (methylamino)-1- phenylpropoxy)phenyl)- 2H-pyrazolo[3,4-d] pyridazin-7-amine | 3.40 | 403.1 | C |
| 28 | 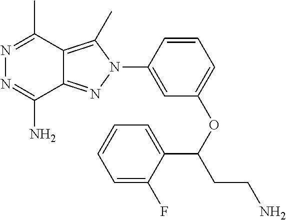 [SMILES: Cc1nnc(N)c2nn(-c3cccc(OC(CCN)c4ccccc4F)c3)c(C)c12] | 2-(3-(3-amino-1-(2- fluorophenyl)propoxy) phenyl)-3,4-dimethyl-2H- pyrazolo[3,4-d] pyridazin-7-amine | 3.34 | 407.0 | C |
| 29 | 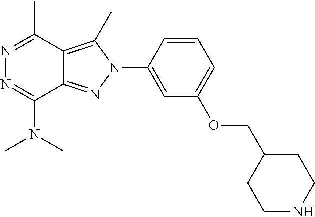 [SMILES: Cc1nnc(N(C)C)c2nn(-c3cccc(OCC4CCNCC4)c3)c(C)c12] | N,N,3,4-tetramethyl-2- (3-(piperidin-4- ylmethoxy)phenyl)-2H- pyrazolo[3,4-d] pyridazin-7-amine | 2.86 | 381.2 | A |

(1) Product was obtained as a pure diastereomer in racemic form (mixture with N,N,3,4-tetramethyl-2-(3-((S)-((R)-morpholin-2-yl)(phenyl)methoxy)phenyl)-2H-pyrazolo[3,4-d]pyridazin-7-amine)
(2) Product was obtained as a pure diastereomer in racemic form (mixture with N,N,3,4-tetramethyl-2-(3-((R)-((R)-morpholin-2-yl)(phenyl)methoxy)phenyl)-2H-pyrazolo[3,4-d]pyridazin-7-amine

### Example 30: 2-(3-(1-(2-Fluorophenyl)-3-(methylamino)propoxy)phenyl)-N,N,3,4-tetramethyl-2H-pyrazolo[3,4-d]pyridazin-7-amine

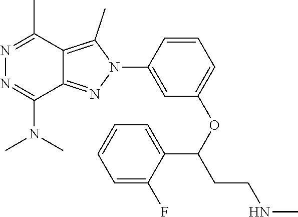 [SMILES: CNCCC(Oc1cccc(-n2nc3c(N(C)C)nnc(C)c3c2C)c1)c1ccccc1F]

Step 1. tert-Butyl (3-(3-(7-chloro-3,4-dimethyl-2H-pyrazolo[3,4-d]pyridazin-2-yl)phenoxy)-3-(2-fluorophenyl)propyl)(methyl)carbamate: A suspension of intermediate 2 (0.57 g, 2.07 mmol), K₂CO₃ (0.86 g, 6.2 mmol) and tert-butyl (3-chloro-3-(2-fluorophenyl)propyl)(methyl)carbamate (prepared following the procedure described in Example 1 Step 1, starting from 1-(2-fluorophenyl)-3-(methylamino)propan-1-ol, 0.81 g, 2.7 mmol) in DMF (5.7 mL) was heated in a sealed tube at 100° C. overnight. Water and EtOAc were added to the cooled reaction mixture and the phases were separated. The aqueous phase was extracted twice with EtOAc. The combined organic phases were washed with brine, dried over Na₂SO₄ and concentrated to dryness to afford the title compound (1.2 g, quant yield), which was used without further purification.

Step 2. tert-Butyl (3-(3-(7-(dimethylamino)-3,4-dimethyl-2H-pyrazolo[3,4-d]pyridazin-2-yl)phenoxy)-3-(2-fluorophenyl)propyl)(methyl)carbamate: A mixture of the product obtained in step 1 (198 mg, 0.36 mmol), TEA (0.1 mL, 0.73 mmol) and dimethylamine (33% in EtOH, 0.07 mL, 0.36 mmol) in IPA (2 mL) was heated in a sealed tube at 80° C. overnight. It was concentrated to dryness and the residue was purified by flash chromatography, silica gel, gradient DCM to MeOH:DCM (1:4) to give the title compound (133 mg, 66% yield).

Step 3. Title compound: Following a similar procedure to the preparation of Example 1 Step 3, and starting from the compound obtained in Step 2, the title compound was obtained (84 mg, 77% yield).

HPLC retention time (method A): 3.88 min; MS: 449.2 (M+H).

This method was used for the preparation of Examples 31-33 using suitable starting materials:

| | | | Ret | | |
|---|---|---|---|---|---|
| | | | time | MS | HPLC |
| EX | Structure | Chemical name | (min) | (M + H) | Method |
| 31 | 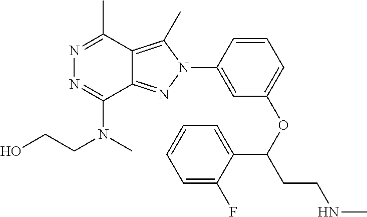 [SMILES: CNCCC(Oc1cccc(-n2nc3c(N(C)CCO)nnc(C)c3c2C)c1)c1ccccc1F] | 2-((2-(3-(1-(2- fluorophenyl)-3- (methylamino)propoxy) phenyl)-3,4-dimethyl- 2H-pyrazolo[3,4-d] pyridazin-7-yl) (methyl)amino)ethanol | 3.43 | 479.2 | A |
| 32 | 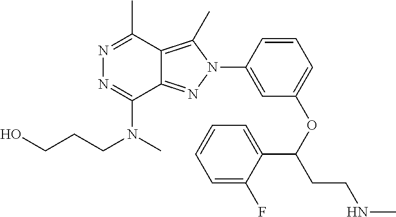 [SMILES: CNCCC(Oc1cccc(-n2nc3c(N(C)CCCO)nnc(C)c3c2C)c1)c1ccccc1F] | 3-((2-(3-(1-(2- fluorophenyl)-3- (methylamino)propoxy) phenyl)-3,4-dimethyl- 2H-pyrazolo[3,4-d] pyridazin-7-yl)(methyl)amino) propan-1-ol | 3.52 | 493.2 | A |
| 33 | 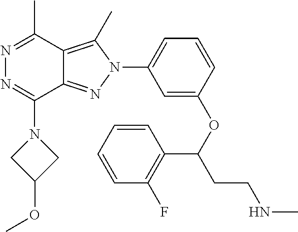 [SMILES: CNCCC(Oc1cccc(-n2nc3c(N4CC(OC)C4)nnc(C)c3c2C)c1)c1ccccc1F] | 3-(2-fluorophenyl)-3-(3- (7-(3-methoxyazetidin- 1-yl)-3,4-dimethyl-2H- pyrazolo[3,4-d] pyridazin-2-yl) phenoxy)-N- methylpropan-1-amine | 3.42 | 491.3 | B |

### Example 34: N,N,3,4-Tetramethyl-2-(3-(3-(methylamino)-1-(thiazol-2-yl)propoxy)phenyl)-2H-pyrazolo[3,4-d]pyridazin-7-amine

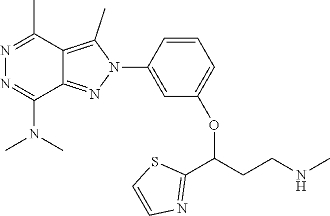 [SMILES: CNCCC(Oc1cccc(-n2nc3c(N(C)C)nnc(C)c3c2C)c1)c1nccs1]

Step 1. tert-Butyl (3-(3-(7-(dimethylamino)-3,4-dimethyl-2H-pyrazolo[3,4-d]pyridazin-2-yl)phenoxy)-3-(thiazol-2-yl)propyl)(methyl)carbamate: In a sealed tube, intermediate 3 (118 mg, 0.41 mmol), tert-butyl (3-hydroxy-3-(thiazol-2-yl)propyl)(methyl)carbamate (prepared following the Boc-protection procedure described in Example 1 Step 1, starting from 3-(methylamino)-1-(thiazol-2-yl)propan-1-ol, 113 mg, 0.41 mmol) and tributylphosphine (0.125 mL, 0.5 mmol) were dissolved in toluene (3.3 mL). Then, ADDP (126 mg, 0.5 mmol) was added and the reaction mixture was heated at 100° C. overnight. After cooling, the mixture was filtered over a pad of celite and the cake was washed with toluene. The filtrate was concentrated to dryness and the residue was purified by flash chromatography, silica gel, gradient DCM to MeOH:DCM (1:4) to give the title compound (129 mg, 57% yield).

Step 2. Title compound: Following a similar procedure to the preparation of Example 1 Step 3, and starting from the compound obtained in Step 1, the title compound was obtained (36 mg, 34% yield).

HPLC retention time (method A): 3.27 min; MS: 438.1 (M+H).

This method was used for the preparation of Examples 35-50 using suitable starting materials:

| | | | Ret | | |
|---|---|---|---|---|---|
| | | | time | MS | HPLC |
| EX | Structure | Chemical name | (min) | (M + H) | Method |
| 35 | 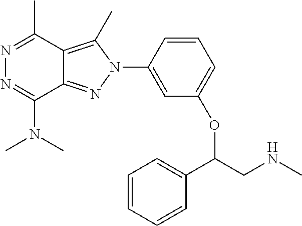 [SMILES: CNCC(Oc1cccc(-n2nc3c(N(C)C)nnc(C)c3c2C)c1)c1ccccc1] | N,N,3,4-tetramethyl-2- (3-(2-(methylamino)-1- phenylethoxy)phenyl)- 2H-pyrazolo[3,4-d] pyridazin-7-amine | 3.78 | 417.2 | A |
| 36 | 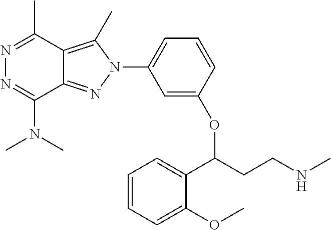 [SMILES: CNCCC(Oc1cccc(-n2nc3c(N(C)C)nnc(C)c3c2C)c1)c1ccccc1OC] | 2-(3-(1-(2- methoxyphenyl)-3- (methylamino)propoxy) phenyl)-N,N,3,4- tetramethyl-2H- pyrazolo[3,4-d] pyridazin-7-amine (*) | 3.74 | 461.2 | A |
| 37 | 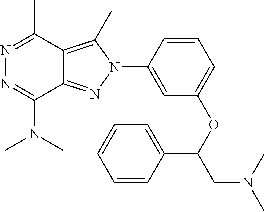 [SMILES: Cc1nnc(N(C)C)c2nn(-c3cccc(OC(CN(C)C)c4ccccc4)c3)c(C)c12] | 2-(3-(2-(dimethylamino)-1- phenylethoxy)phenyl)- N,N,3,4-tetramethyl- 2H-pyrazolo[3,4-d] pyridazin-7-amine | 4.06 | 431.2 | A |
| 38 | 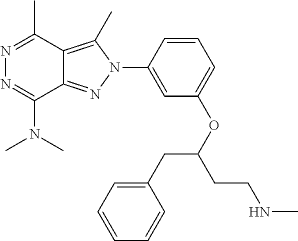 [SMILES: CNCCC(Cc1ccccc1)Oc1cccc(-n2nc3c(N(C)C)nnc(C)c3c2C)c1] | N,N,3,4-tetramethyl-2- (3-((4-(methylamino)-1- phenylbutan-2- yl)oxy)phenyl)-2H- pyrazolo[3,4-d] pyridazin-7-amine | 4.33 | 445.2 | A |
| 39 | 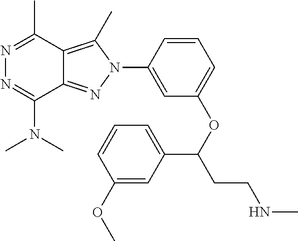 [SMILES: CNCCC(Oc1cccc(-n2nc3c(N(C)C)nnc(C)c3c2C)c1)c1cccc(OC)c1] | 2-(3-(1-(3- methoxyphenyl)-3- (methylamino)propoxy) phenyl)-N,N,3,4- tetramethyl-2H- pyrazolo[3,4-d] pyridazin-7-amine (*) | 4.07 | 461.2 | A |
| 40 | 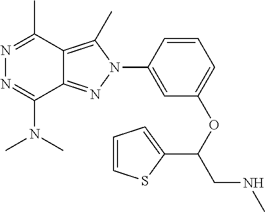 [SMILES: CNCC(Oc1cccc(-n2nc3c(N(C)C)nnc(C)c3c2C)c1)c1cccs1] | N,N,3,4-tetramethyl-2- (3-(2-(methylamino)-1- (thiophen-2- yl)ethoxy)phenyl)-2H- pyrazolo[3,4-d] pyridazin-7-amine (*) | 3.72 | 423.2 | A |
| 41 | 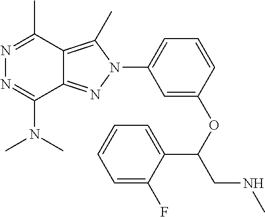 [SMILES: CNCC(Oc1cccc(-n2nc3c(N(C)C)nnc(C)c3c2C)c1)c1ccccc1F] | 2-(3-(1-(2- fluorophenyl)-2- (methylamino)ethoxy) phenyl)-N,N,3,4- tetramethyl-2H- pyrazolo[3,4-d] pyridazin-7-amine | 3.88 | 435.2 | A |
| 42 | 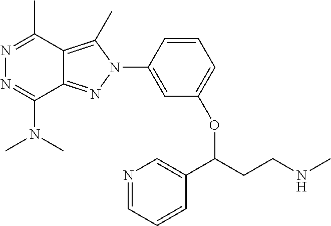 [SMILES: CNCCC(Oc1cccc(-n2nc3c(N(C)C)nnc(C)c3c2C)c1)c1cccnc1] | N,N,3,4-tetramethyl-2- (3-(3-(methylamino)-1- (pyridin-3- yl)propoxy)phenyl)-2H- pyrazolo[3,4-d] pyridazin-7-amine | 3.33 | 432.2 | A |
| 43 | 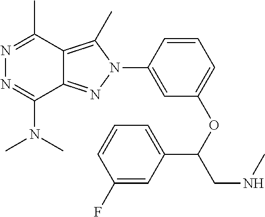 [SMILES: CNCC(Oc1cccc(-n2nc3c(N(C)C)nnc(C)c3c2C)c1)c1cccc(F)c1] | 2-(3-(1-(3- fluorophenyl)-2- (methylamino)ethoxy) phenyl)-N,N,3,4- tetramethyl-2H- pyrazolo[3,4-d] pyridazin-7-amine | 3.91 | 435.2 | A |
| 44 | 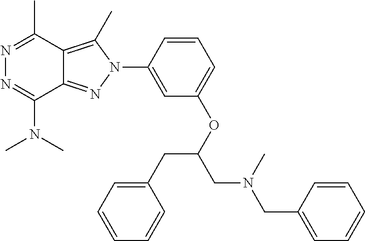 [SMILES: Cc1nnc(N(C)C)c2nn(-c3cccc(OC(Cc4ccccc4)CN(C)Cc4ccccc4)c3)c(C)c12] | 2-(3-((1- (benzyl(methyl)amino)- 3-phenylpropan-2- yl)oxy)phenyl)-N,N,3,4- tetramethyl-2H- pyrazolo[3,4-d] pyridazin-7-amine | 5.55 | 521.3 | A |
| 45 | 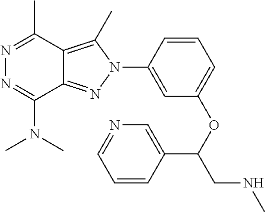 [SMILES: CNCC(Oc1cccc(-n2nc3c(N(C)C)nnc(C)c3c2C)c1)c1cccnc1] | N,N,3,4-tetramethyl-2- (3-(2-(methylamino)-1- (pyridin-3- yl)ethoxy)phenyl)-2H- pyrazolo[3,4-d] pyridazin-7-amine | 3.31 | 418.1 | A |
| 46 | 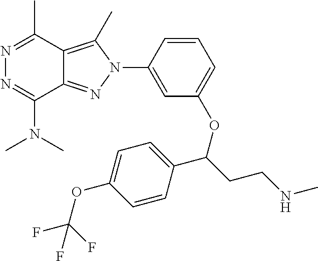 [SMILES: CNCCC(Oc1cccc(-n2nc3c(N(C)C)nnc(C)c3c2C)c1)c1ccc(OC(F)(F)F)cc1] | N,N,3,4-tetramethyl-2- (3-(3-(methylamino)-1- (4-(trifluoromethoxy) phenyl)propoxy) phenyl)-2H- pyrazolo[3,4-d] pyridazin-7-amine | 4.73 | 515.2 | A |
| 47 | 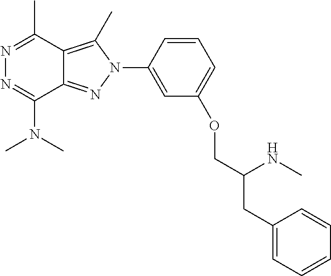 [SMILES: CNC(COc1cccc(-n2nc3c(N(C)C)nnc(C)c3c2C)c1)Cc1ccccc1] | N,N,3,4-tetramethyl-2- (3-(2-(methylamino)-3- phenylpropoxy)phenyl)- 2H-pyrazolo[3,4-d] pyridazin-7-amine | 4.1 | 431.2 | A |
| 48 | 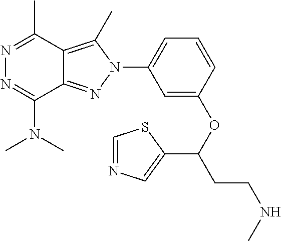 [SMILES: CNCCC(Oc1cccc(-n2nc3c(N(C)C)nnc(C)c3c2C)c1)c1cncs1] | N,N,3,4-tetramethyl-2- (3-(3-(methylamino)-1- (thiazol-5- yl)propoxy)phenyl)-2H- pyrazolo[3,4-d] pyridazin-7-amine | 3.71 | 438.3 | A |
| 49 | 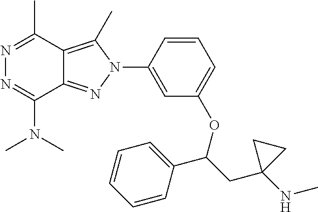 [SMILES: CNC1(CC(Oc2cccc(-n3nc4c(N(C)C)nnc(C)c4c3C)c2)c2ccccc2)CC1] | N,N,3,4-tetramethyl-2- (3-(2-(1- (methylamino) cyclopropyl)-1- phenylethoxy)phenyl)- 2H-pyrazolo[3,4-d] pyridazin-7-amine | 4.48 | 457.2 | B |
| 50 | 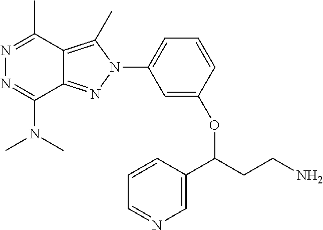 [SMILES: Cc1nnc(N(C)C)c2nn(-c3cccc(OC(CCN)c4cccnc4)c3)c(C)c12] | 2-(3-(3-amino-1- (pyridin-3- yl)propoxy)phenyl)- N,N,3,4-tetramethyl- 2H-pyrazolo[3,4-d] pyridazin-7-amine | 2.93 | 418.2 | B |

(*) Alternatively, deprotection was carried out by treating a solution of the Boc-protected compound in ACN with 2 eq of iodotrimethylsilane at RT.

### Example 51: N,N,3,4-Tetramethyl-2-(3-(3-(methylamino)-1-(thiophen-2-yl)propoxy)phenyl)-2H-pyrazolo[3,4-d]pyridazin-7-amine

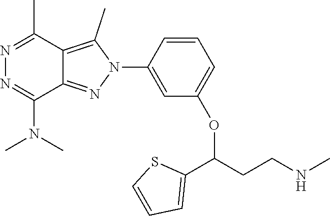 [SMILES: CNCCC(Oc1cccc(-n2nc3c(N(C)C)nnc(C)c3c2C)c1)c1cccs1]

Step 1. 2-(1,3-Dichloropropyl)thiophene. TEA (1.43 mL, 10.3 mmol) and methanesulfonyl chloride (0.62 mL, 8.0 mmol) were slowly added to a solution of 3-chloro-1-(thiophen-2-yl)propan-1-ol (1.01 g, 5.72 mmol) in DCM (34 mL), previously cooled at 0-5° C. and the mixture was stirred at this temperature overnight. Sat. NaHCO₃ was added and the phases were separated. The aqueous phase was back extracted twice with DCM. The combined organic phases were washed with brine, dried over MgSO₄ and concentrated to dryness to afford the title compound (1.16 g, quant yield) as a crude product that was used without further purification.

Step 2. 2-(3-(3-Chloro-1-(thiophen-2-yl)propoxy)phenyl)-N,N,3,4-tetramethyl-2H-pyrazolo[3,4-d]pyridazin-7-amine. The product was prepared following the alkylation procedure described in Step 1 of Example 1, starting from Intermediate 3 (0.6 g, 1.87 mmol) and the product obtained in step 1 (0.43 g, 2.25 mmol). After purification by flash chromatography (silica gel, gradient DCM to MeOH:DCM (1:4)), the title compound was obtained (100 mgs, 12% yield).

Step 3. Title compound: In a sealed tube, a mixture of the product obtained in Step 1 (100 mg, 0.226 mmol) and methylamine (33 wt % in EtOH, 5 mL) were heated at 50° C. overnight. The solvent was concentrated, the residue was dissolved in DCM and the organic phase was washed with 1 M NaOH solution. The organic phase was dried over sodium sulfate and concentrated under vacuum. The crude product was purified by flash chromatography, silica gel, gradient DCM to MeOH:DCM (1:4) to give the title compound (3 mg, 3% yield).

HPLC retention time (method A): 3.68 min; MS: 437.2 (M+H).

### Example 52: 2-(3-(2-(Dimethylamino)-1-(pyridin-3-yl)ethoxy)phenyl)-N,N,3,4-tetramethyl-2H-pyrazolo[3,4-d]pyridazin-7-amine

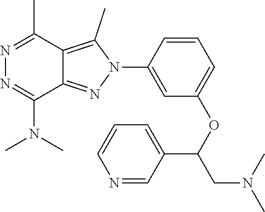 [SMILES: Cc1nnc(N(C)C)c2nn(-c3cccc(OC(CN(C)C)c4cccnc4)c3)c(C)c12]

To a solution of Example 45 (32 mg, 0.076 mmol) in DCM (2 mL), formaldehyde (37 wt % in water, 27 μL, 0.38 mmol) was added. The mixture was stirred at r.t. for 45 min, then sodium tris(acetoxy)borohydride (22 mg, 0.107 mmol) was added and the reaction mixture was stirred at r.t. overnight. It was then diluted with DCM that was washed with a saturated solution of NaHCO₃ and then brine. The organic phase was dried over sodium sulfate and concentrated to dryness. The residue was purified by flash chromatography, silica gel, gradient DCM to MeOH:DCM (1:4) to give the title compound (16 mg, 50% yield).

HPLC retention time (method A): 3.44 min; MS: 432.2 (M+H).

### Example 53. N,N,3,4-Tetramethyl-2-(3-((1-phenethylpiperidin-4-yl)methoxy)phenyl)-2H-pyrazolo[3,4-d]pyridazin-7-amine

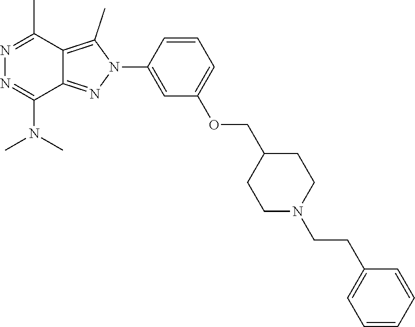 [SMILES: Cc1nnc(N(C)C)c2nn(-c3cccc(OCC4CCN(CCc5ccccc5)CC4)c3)c(C)c12]

To a solution of Example 29 (70 mg, 0.184 mmol) in THF (2 mL), 2-phenylacetaldehyde (28 μL, 0.24 mmol) was added. The mixture was stirred for 15 min at r.t. and then sodium tris(acetoxy)borohydride (120 mg, 0.55 mmol) and AcOH (11 μL, 0.184 mmol) were added. The reaction mixture was stirred at r.t. overnight. Then, 1 M NaOH solution was added and it was extracted with EtOAc. The combined organic phases were washed with brine, dried over sodium sulfate and concentrated to dryness. The residue was purified by flash chromatography, silica gel, gradient DCM to MeOH:DCM (1:4) to give the title compound (24 mg, 27% yield).

HPLC retention time (method A): 4.1 min; MS: 485.3 (M+H).

This method was used for the preparation of Examples 54-55 using suitable starting materials:

| | | | Ret | | |
|---|---|---|---|---|---|
| | | | time | MS | HPLC |
| EX | Structure | Chemical name | (min) | (M + H) | Method |
| 54 | 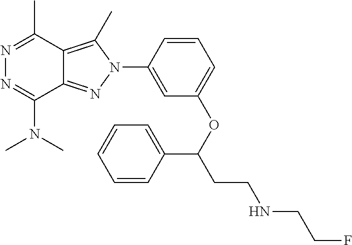 [SMILES: Cc1nnc(N(C)C)c2nn(-c3cccc(OC(CCNCCF)c4ccccc4)c3)c(C)c12] | 2-(3-(3-((2- fluoroethyl)amino)-1- phenylpropoxy)phenyl)- N,N,3,4-tetramethyl- 2H-pyrazolo[3,4-d] pyridazin-7-amine | 4.16 | 463.2 | B |
| 55 | 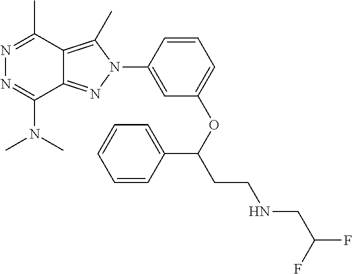 [SMILES: Cc1nnc(N(C)C)c2nn(-c3cccc(OC(CCNCC(F)F)c4ccccc4)c3)c(C)c12] | 2-(3-(3-((2,2- difluoroethyl)amino)-1- phenylpropoxy)phenyl)- N,N,3,4-tetramethyl- 2H-pyrazolo[3,4-d] pyridazin-7-amine | 4.51 | 481.2 | B |

### Example 56. 3-(1-(3-(7-(Dimethylamino)-3,4-dimethyl-2H-pyrazolo[3,4-d]pyridazin-2-yl)phenoxy)-3-(methylamino)propyl)benzamide

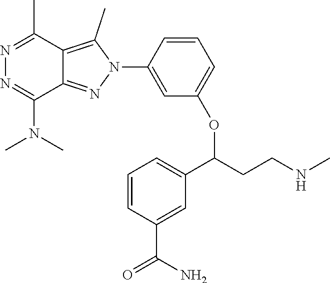 [SMILES: CNCCC(Oc1cccc(-n2nc3c(N(C)C)nnc(C)c3c2C)c1)c1cccc(C(N)=O)c1]

To a solution of Example 11 (123 mg, 0.27 mmol) in tert-butanol (1 mL), KOH (151 mg, 2.7 mmol) was added and the mixture was heated at 80° C. overnight. Then, it was concentrated to dryness and the residue was purified by flash chromatography, silica gel, gradient DCM to MeOH:DCM:conc ammonia (1:4:0.15) to give the title compound (109 mg, 85% yield).

HPLC retention time (method A): 3.28 min; MS: 474.2 (M+H).

This method was used for the preparation of Example 57 starting from Example 7:

| | | | Ret | | |
|---|---|---|---|---|---|
| | | | time | MS | HPLC |
| EX | Structure | Chemical name | (min) | (M + H) | Method |
| 57 | 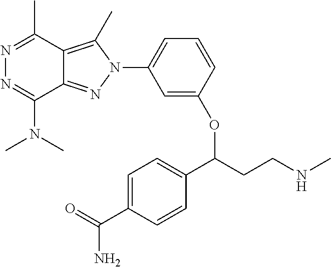 [SMILES: CNCCC(Oc1cccc(-n2nc3c(N(C)C)nnc(C)c3c2C)c1)c1ccc(C(N)=O)cc1] | 4-(1-(3-(7- (dimethylamino)-3,4- dimethyl-2H- pyrazolo[3,4-d] pyridazin-2- yl)phenoxy)-3- (methylamino)propyl) benzamide | 3.22 | 474.1 | A |

### Examples 58 and 59: (S)-N,N,3,4-Tetramethyl-2-(3-(3-(methylamino)-1-phenylpropoxy)phenyl)-2H-pyrazolo[3,4-d]pyridazin-7-amine and (R)-N,N,3,4-tetramethyl-2-(3-(3-(methylamino)-1-phenylpropoxy)phenyl)-2H-pyrazolo[3,4-d]pyridazin-7-amine

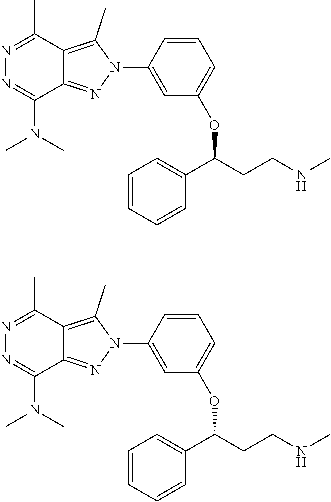 [SMILES: CNCC[C@H](Oc1cccc(-n2nc3c(N(C)C)nnc(C)c3c2C)c1)c1ccccc1 ; CNCC[C@@H](Oc1cccc(-n2nc3c(N(C)C)nnc(C)c3c2C)c1)c1ccccc1]

Starting from Example 1, a chiral preparative HPLC separation (column: Chiralpak ASH; temperature: ambient; flow: 10 mL/min; eluent: n-Heptane/IPA 80/20 v/v+0.2% DEA) was carried out to give the title compounds.

### Examples 60 and 61: (R)-N,N,3,4-Tetramethyl-2-(3-(2-(methylamino)-1-phenylethoxy)phenyl)-2H-pyrazolo[3,4-d]pyridazin-7-amine and (S)-N,N,3,4-tetramethyl-2-(3-(2-(methylamino)-1-phenylethoxy)phenyl)-2H-pyrazolo[3,4-d]pyridazin-7-amine

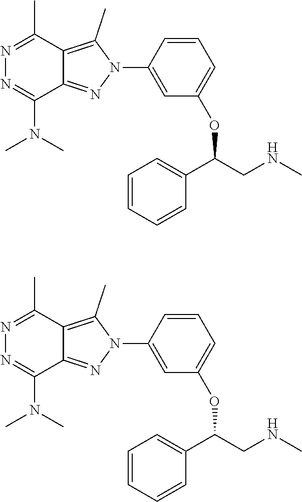 [SMILES: CNC[C@H](Oc1cccc(-n2nc3c(N(C)C)nnc(C)c3c2C)c1)c1ccccc1 ; CNC[C@@H](Oc1cccc(-n2nc3c(N(C)C)nnc(C)c3c2C)c1)c1ccccc1]

Starting from Example 35, a chiral preparative HPLC separation (column: Chiralpak IA; temperature: ambient; flow: 20 mL/min; eluent: (IPA+0.2% DEA)/EtOH 60/40 v/v) was carried out to give the title compounds.

### Examples 62 and 63: (R)-2-(3-(1-(2-Fluorophenyl)-3-(methylamino)propoxy)phenyl)-N,N,3,4-tetramethyl-2H-pyrazolo[3,4-d]pyridazin-7-amine and (S)-2-(3-(1-(2-fluorophenyl)-3-(methylamino)propoxy)phenyl)-N,N,3,4-tetramethyl-2H-pyrazolo[3,4-d]pyridazin-7-amine

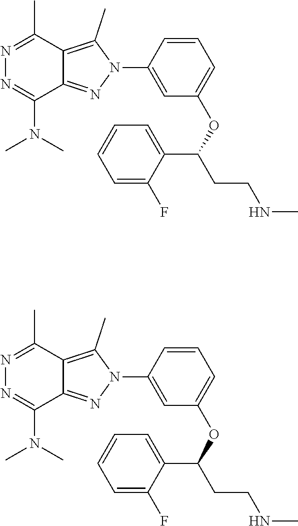 [SMILES: CNCC[C@@H](Oc1cccc(-n2nc3c(N(C)C)nnc(C)c3c2C)c1)c1ccccc1F ; CNCC[C@H](Oc1cccc(-n2nc3c(N(C)C)nnc(C)c3c2C)c1)c1ccccc1F]

Starting from Example 30, a chiral preparative HPLC separation (column: Chiralpak IA; temperature: ambient; flow: 5 mL/min; eluent: IPA/(EtOH+0.2% DEA) 50/50 v/v) was carried out to give the title compounds.

### Examples 64 and 65: (S)-N,N,3,4-tetramethyl-2-(3-(3-(methylamino)-1-(pyridin-3-yl)propoxy)phenyl)-2H-pyrazolo[3,4-d]pyridazin-7-amine and (R)-N,N,3,4-tetramethyl-2-(3-(3-(methylamino)-1-(pyridin-3-yl)propoxy)phenyl)-2H-pyrazolo[3,4-d]pyridazin-7-amine

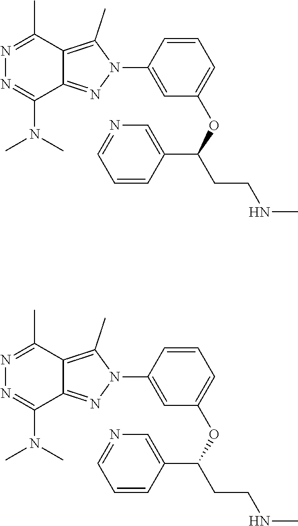 [SMILES: CNCC[C@H](Oc1cccc(-n2nc3c(N(C)C)nnc(C)c3c2C)c1)c1cccnc1 ; CNCC[C@@H](Oc1cccc(-n2nc3c(N(C)C)nnc(C)c3c2C)c1)c1cccnc1]

Starting from Example 42, a chiral preparative HPLC separation (column: Chiralpak IA; temperature: ambient; flow: 5 mL/min; eluent: MeOH+0.2% DEA) was carried out to give the title compounds.

Following the method described for the preparation of Example 1 but starting from Intermediate 7 and suitable starting materials, Examples 66 and 67 were obtained:

| | | | Ret | | |
|---|---|---|---|---|---|
| | | | time | MS | HPLC |
| EX | Structure | Chemical name | (min) | (M + H) | Method |
| 66 | 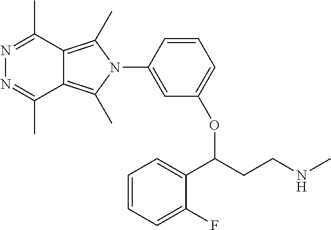 [SMILES: CNCCC(Oc1cccc(-n2c(C)c3c(C)nnc(C)c3c2C)c1)c1ccccc1F] | 3-(2-fluorophenyl)-N- methyl-3-(3-(1,4,5,7- tetramethyl-6H- pyrrolo[3,4-d]pyridazin- 6-yl)phenoxy)propan-1- amine | 4.02 | 433.2 | C |
| 67 | 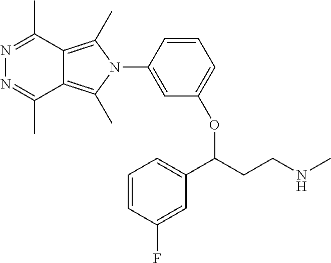 [SMILES: CNCCC(Oc1cccc(-n2c(C)c3c(C)nnc(C)c3c2C)c1)c1cccc(F)c1] | 3-(3-fluorophenyl)-N- methyl-3-(3-(1,4,5,7- tetramethyl-6H- pyrrolo[3,4-d]pyridazin- 6-yl)phenoxy)propan-1- amine | 4.09 | 433.2 | C |

### Examples 68 and 69: (S)-3-(3-Fluorophenyl)-N-methyl-3-(3-(1,4,5,7-tetramethyl-6H-pyrrolo[3,4-d]pyridazin-6-yl)phenoxy)propan-1-amine and (R)-3-(3-fluorophenyl)-N-methyl-3-(3-(1,4,5,7-tetramethyl-6H-pyrrolo[3,4-d]pyridazin-6-yl)phenoxy)propan-1-amine

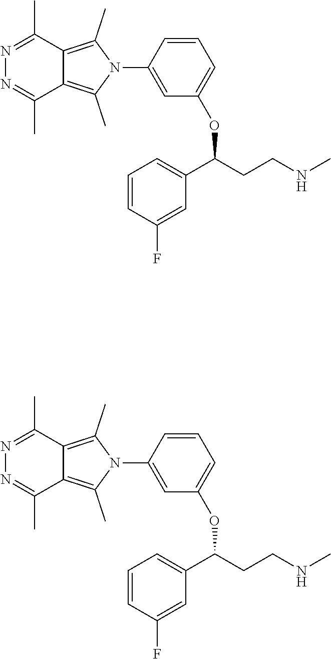 [SMILES: CNCC[C@H](Oc1cccc(-n2c(C)c3c(C)nnc(C)c3c2C)c1)c1cccc(F)c1 ; CNCC[C@@H](Oc1cccc(-n2c(C)c3c(C)nnc(C)c3c2C)c1)c1cccc(F)c1]

Starting from Example 67, a chiral preparative SFC separation (column: Chiralpak IG; temperature: 40° C.; flow: 50 mL/min; BPR: 100 BarG; eluent: EtOH:CO₂ 35:65 (0.2% v/v DEA)) was carried out to give the title compounds.

Table of Examples with binding to the NET Receptor and the α₂δ-1 Subunit of the voltage-gated calcium channel:

### Biological Activity

### Pharmacological Study

Human α₂δ-1 Subunit of Caᵥ2.2 Calcium Channel Assay

Human α₂δ-1 enriched membranes (2.5 μg) were incubated with 15 nM of radiolabeled [3H]-Gabapentin in assay buffer containing Hepes-KOH 10 mM, pH 7.4. NSB (non specific binding) was measured by adding 10 μM pregabalin. The binding of the test compound was measured at five different concentrations. After 60 min incubation at 27° C., binding reaction was terminated by filtering through Multiscreen GF/C (Millipore) presoaked in 0.5% polyethyleneimine in Vacuum Manifold Station, followed by 3 washes with ice-cold filtration buffer containing 50 mM Tris-HCl, pH 7.4. Filter plates were dried at 60° C. for 1 hour and 30 μl of scintillation cocktail were added to each well before radioactivity reading. Readings were performed in a Trilux 1450 Microbeta radioactive counter (Perkin Elmer).

### Binding Assay to Human Norepinephrine Transporter (NET).

Human norepinephrine transporter (NET) enriched membranes (5 μg) were incubated with 5 nM of radiolabeled [3H]-Nisoxetin in assay buffer containing 50 mM Tris-HCl, 120 mM NaCl, 5 mM KCl, pH 7.4. NSB (non specific binding) was measured by adding 10 μM of desipramine. The binding of the test compound was measured at five different concentrations. After 60 min incubation at 4° C., binding reaction was terminated by filtering through Multiscreen GF/C (Millipore) presoaked in 0.5% polyethyleneimine in Vacuum Manifold Station, followed by 3 washes with ice-cold filtration buffer containing 50 mM Tris-HCl, 0.9% NaCl, pH 7.4. Filter plates were dried at 60° C. for 1 hour and 30 μl of scintillation cocktail were added to each well before radioactivity reading. Readings were performed in a Trilux 1450 Microbeta radioactive counter (Perkin Elmer).

### Results:

As this invention is aimed at providing a compound or a chemically related series of compounds which act as dual ligands of the α₂δ subunit of voltage-gated calcium channels and the NET receptor it is a very preferred embodiment in which the compounds are selected which act as dual ligands of the α₂δ subunit of voltage-gated calcium channels and the NET receptor and especially compounds which have a binding expressed as Kᵢ responding to the following scales:

Kᵢ(NET) is preferably <1000 nM, more preferably <500 nM, even more preferably <100 nM.
Kᵢ(α₂δ-1) is preferably <10000 nM, more preferably <5000 nM, or even more preferably <500 nM.

The following scale has been adopted for representing the binding to the NET receptor expressed as Kᵢ:
+Ki-NET>=1000 nM
++500 nM<Ki-NET<1000 nM
+++100 nM<Ki-NET<500 nM
++++Ki-NET<100 nM

The following scale has been adopted for representing the binding to the α₂δ-1 subunit of voltage-gated calcium channels expressed as Kᵢ:
+Kᵢ(α₂δ-1)>=5000 nM
++500 nM<=Kᵢ(α₂δ-1)<5000 nM
+++Kᵢ(α₂δ-1)<500 nM

All compounds prepared in the present application exhibit binding to the α₂δ-1 subunit of voltage-gated calcium channels and the NET receptor, in particular the following binding results are shown:

| | Binding | Binding |
|---|---|---|
| Example | NET | α2δ-1 |
| 1 | ++++ | ++ |
| 2 | +++ | ++ |
| 3 | +++ | ++ |
| 4 | +++ | ++ |
| 5 | ++++ | ++ |
| 6 | +++ | ++ |
| 7 | +++ | ++ |
| 8 | ++++ | ++ |
| 9 | +++ | ++ |
| 10 | ++++ | ++ |
| 11 | ++++ | ++ |
| 12 | +++ | ++ |
| 13 | ++ | ++ |
| 14 | +++ | ++ |
| 15 | ++++ | ++ |
| 16 | + | ++ |
| 17 | + | ++ |
| 18 | +++ | ++ |
| 19 | +++ | ++ |
| 20 | +++ | ++ |
| 21 | +++ | ++ |
| 22 | ++++ | ++ |
| 23 | +++ | ++ |
| 24 | ++ | ++ |
| 25 | +++ | ++ |
| 26 | +++ | ++ |
| 27 | ++++ | ++ |
| 28 | ++ | ++ |
| 29 | + | + |
| 30 | ++++ | ++ |
| 31 | ++++ | ++ |
| 32 | ++++ | ++ |
| 33 | ++ | ++ |
| 34 | + | +++ |
| 35 | ++++ | ++ |
| 36 | +++ | ++ |
| 37 | +++ | ++ |
| 38 | +++ | ++ |
| 39 | ++++ | ++ |
| 40 | ++ | ++ |
| 41 | +++ | ++ |
| 42 | +++ | +++ |
| 43 | ++++ | ++ |
| 44 | + | + |
| 45 | + | ++ |
| 46 | +++ | ++ |
| 47 | ++++ | +++ |
| 48 | ++ | +++ |
| 49 | ++++ | ++ |
| 50 | + | ++ |
| 51 | ++++ | ++ |
| 52 | + | ++ |
| 53 | +++ | ++ |
| 54 | +++ | ++ |
| 55 | ++ | ++ |
| 56 | +++ | ++ |
| 57 | +++ | ++ |
| 58 | ++++ | ++ |
| 59 | +++ | ++ |
| 60 | +++ | ++ |
| 61 | ++++ | ++ |
| 62 | ++++ | ++ |
| 63 | ++++ | ++ |
| 64 | +++ | +++ |
| 65 | ++ | ++ |
| 66 | ++ | ++ |
| 67 | +++ | ++ |
| 68 | +++ | + |
| 69 | +++ | +++ |

1-14. (canceled)

15. A compound of general formula (I): 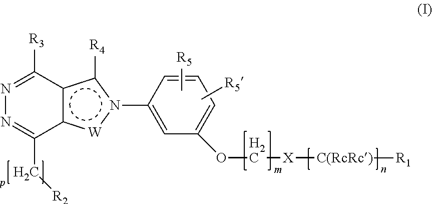
wherein
m is 0, 1, 2, 3 or 4;
n is 0, 1, 2, 3 or 4;
p is 0, 1, 2, 3 or 4;
W is nitrogen or —C(R_{4′})—;
X is selected from the group consisting of a bond, substituted or unsubstituted aryl or —C(RₓR_{x′})—;
Rₓ is selected from the group consisting of substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
R_{x′} is selected from the group consisting of hydrogen, substituted or unsubstituted C₁₋₆ alkyl, substituted or unsubstituted C₂₋₆ alkenyl and substituted or unsubstituted C₂₋₆ alkynyl;
R₁ is selected from the group consisting of —NR₆R_{6′} and substituted or unsubstituted N-containing-heterocyclyl;
wherein R₆ and R_{6′} are independently selected from the group consisting of hydrogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
R₂ is selected from the group consisting of hydrogen, —NR₇R_{7′}, —CN, —CHR₇R_{7′} and substituted or unsubstituted heterocyclyl;
wherein R₇ and R_{7′} are independently selected from the group consisting of hydrogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
R₃ is selected from the group consisting of hydrogen, substituted or unsubstituted C₁₋₆ alkyl, substituted or unsubstituted C₂₋₆ alkenyl and substituted or unsubstituted C₂₋₆ alkynyl;
R₄ is selected from the group consisting of hydrogen, substituted or unsubstituted C₁₋₆ alkyl, substituted or unsubstituted C₂₋₆ alkenyl and substituted or unsubstituted C₂₋₆ alkynyl;
R_{4′} is selected from the group consisting of hydrogen, substituted or unsubstituted C₁₋₆ alkyl, substituted or unsubstituted C₂₋₆ alkenyl and substituted or unsubstituted C₂₋₆ alkynyl;
R₅ and R_{5′} are independently selected from the group consisting of hydrogen, halogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl, substituted or unsubstituted alkyheterocyclyl, —OR₈, —NO₂, —NR₈R_{8′}, —NR₈C(O)R_{8′}, —NR₈S(O)₂R_{8′}, —S(O)₂NR₈R_{8′}, —NR₈C(O)NR_{8′}R_{8″}, —SR₈, —S(O)R₈, S(O)₂R₈, —CN, haloalkyl, haloalkoxy, —C(O)OR₈, —C(O)NR₈R_{8′}, —OCH₂CH₂OR₈, —NR₈S(O)₂NR_{8′}R_{8″} and C(CH₃)₂OR₈;
wherein R₈, R_{8′} and R_{8″} are independently selected from the group consisting of hydrogen, unsubstituted C₁₋₆ alkyl, unsubstituted C₂₋₆ alkenyl, and unsubstituted C₂₋₆ alkynyl;
R_{c} and R_{c′} are independently selected from the group consisting of hydrogen, substituted or unsubstituted C₁₋₆ alkyl, substituted or unsubstituted C₂₋₆ alkenyl and substituted or unsubstituted C₂₋₆ alkynyl;
alternatively, R_{c} and R_{c′}, together with the carbon atom to which they are attached, form a substituted or unsubstituted cycloalkyl;
optionally as a stereoisomer, including enantiomers and diastereomers, a racemate or as a mixture of at least two stereoisomers, including enantiomers and/or diastereomers, in any mixing ratio, or a corresponding salt thereof, or a corresponding solvate thereof.

16. The compound according to claim 15, wherein the compound of Formula (I) is a compound of Formula (I′)

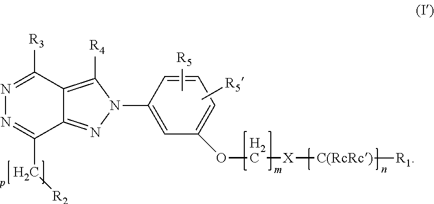

17. The compound according to claim 15, wherein the compound of Formula (I) is a compound of Formula (I²′):

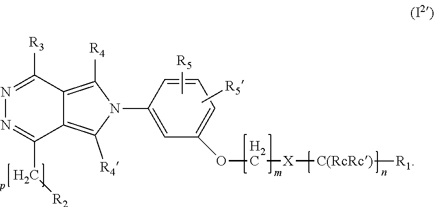

18. The compound according claim 15, wherein
R₁ is selected from the group consisting of —NR₆R_{6′} and substituted or unsubstituted N-containing-heterocyclyl,
wherein R₆ and R_{6′}R₆ and R_{6′} are independently selected from the group consisting of hydrogen, substituted or unsubstituted C₁₋₆ alkyl and substituted or unsubstituted alkylaryl.

19. The compound according to claim 18, wherein R₁ is:
—NR₆R_{6′}, wherein R₆ and R_{6′} are independently selected from the group consisting of hydrogen, substituted or unsubstituted C₁₋₆ alkyl and substituted or unsubstituted alkylaryl, or
an N-containing-heterocyclyl selected from the group consisting of: 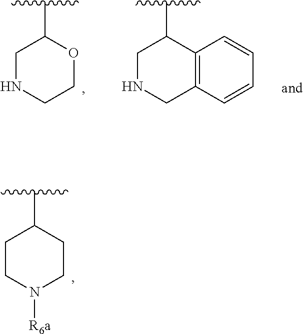
wherein R₆ₐ is substituted or unsubstituted alkylaryl.

20. The compound according to claim 15, wherein
R₂ is selected from the group consisting of —NR₇R_{7′}, and substituted or unsubstituted heterocyclyl;
wherein R₇ and R_{7′} are independently selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl.

21. The compound according to claim 20, wherein R₇ and R_{7′} are independently selected from hydrogen and substituted or unsubstituted C₁₋₆ alkyl.

22. The compound according to claim 15, wherein
X is selected from the group consisting of a bond, substituted or unsubstituted aryl and —C(RₓR_{x′})—, wherein
Rₓ is selected from the group consisting of substituted or unsubstituted aryl, substituted or unsubstituted alkylaryl and substituted or unsubstituted heterocyclyl; and
R_{x′} is hydrogen.

23. The compound according to claim 22, wherein X is a bond or —C(RₓR_{x′})—,
wherein
R_{x′} is hydrogen, and
R_{X} is a group selected from the group consisting of: 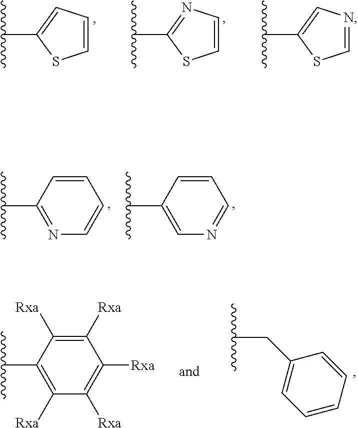
wherein Rₓₐ is independently selected from the group consisting of hydrogen, halogen, —OR₁₀, —CN, haloalkoxy and —C(O)NR₁₀R_{10′}, wherein R₁₀ and R_{10′} independently represent hydrogen or substituted or unsubstituted C₁₋₆ alkyl.

24. The compound according to claim 15, which is selected from the group consisting of:
N,N,3,4-Tetramethyl-2-(3-(3-(methylamino)-1-phenylpropoxy)phenyl)-2H-pyrazolo[3,4-d]pyridazin-7-amine,
2-(3-(3-(dimethylamino)-1-phenylpropoxy)phenyl)-N,N,3,4-tetramethyl-2H-pyrazolo[3,4-d]pyridazin-7-amine,
2-(3-(3-(ethylamino)-1-phenylpropoxy)phenyl)-N,N,3,4-tetramethyl-2H-pyrazolo[3,4-d]pyridazin-7-amine,
2-(3-(3-amino-1-phenylpropoxy)phenyl)-N,N,3,4-tetramethyl-2H-pyrazolo[3,4-d]pyridazin-7-amine,
2-(3-(1-(3-fluorophenyl)-3-(methylamino)propoxy)phenyl)-N,N,3,4-tetramethyl-2H-pyrazolo[3,4-d]pyridazin-7-amine,
N,N,3,4-tetramethyl-2-(3-(4-(methylamino)-1-phenylbutoxy)phenyl)-2H-pyrazolo[3,4-d]pyridazin-7-amine,
4-(1-(3-(7-(dimethylamino)-3,4-dimethyl-2H-pyrazolo[3,4-d]pyridazin-2-yl)phenoxy)-3-(methylamino)propyl)benzonitrile,
2-(3-(1-(4-fluorophenyl)-3-(methylamino)propoxy)phenyl)-N,N,3,4-tetramethyl-2H-pyrazolo[3,4-d]pyridazin-7-amine,
N,N,3,4-tetramethyl-2-(3-(3-(methylamino)-1-(pyridin-2-yl)propoxy)phenyl)-2H-pyrazolo[3,4-d]pyridazin-7-amine,
N,N,3,4-tetramethyl-2-(3-(3-(methylamino)-1-(pyridin-4-yl)propoxy)phenyl)-2H-pyrazolo[3,4-d]pyridazin-7-amine,
3-(1-(3-(7-(dimethylamino)-3,4-dimethyl-2H-pyrazolo[3,4-d]pyridazin-2-yl)phenoxy)-3-(methylamino)propyl)benzonitrile,
N,N,3,4-tetramethyl-2-(3-((R)-((S)-morpholin-2-yl)(phenyl)methoxy)phenyl)-2H-pyrazolo[3,4-d]pyridazin-7-amine,
N,N,3,4-tetramethyl-2-(3-((S)-((S)-morpholin-2-yl)(phenyl)methoxy)phenyl)-2H-pyrazolo[3,4-d]pyridazin-7-amine,
2-(3-(3-amino-3-phenylpropoxy)phenyl)-N,N,3,4-tetramethyl-2H-pyrazolo[3,4-d]pyridazin-7-amine,
N,N,3,4-tetramethyl-2-(3-(2-(methylamino)-2-phenylethoxy)phenyl)-2H-pyrazolo[3,4-d]pyridazin-7-amine,
N,N,3,4-tetramethyl-2-(3-((1,2,3,4-tetrahydroisoquinolin-4-yl)oxy)phenyl)-2H-pyrazolo[3,4-d]pyridazin-7-amine,
2-(3-(3-(ethylamino)-1-(3-fluorophenyl)propoxy)phenyl)-N,N,3,4-tetramethyl-2H-pyrazolo[3,4-d]pyridazin-7-amine,
2-(3-(1-(3,5-difluorophenyl)-3-(methylamino)propoxy)phenyl)-N,N,3,4-tetramethyl-2H-pyrazolo[3,4-d]pyridazin-7-amine,
2-(3-(3-amino-1-(2-fluorophenyl)propoxy)phenyl)-N,N,3,4-tetramethyl-2H-pyrazolo[3,4-d]pyridazin-7-amine,
2-(3-(3-amino-1-(4-fluorophenyl)propoxy)phenyl)-N,N,3,4-tetramethyl-2H-pyrazolo[3,4-d]pyridazin-7-amine,
2-(3-(3-amino-1-(3-fluorophenyl)propoxy)phenyl)-N,N,3,4-tetramethyl-2H-pyrazolo[3,4-d]pyridazin-7-amine,
2-(3-(1-(2-fluorophenyl)-3-(methylamino)propoxy)phenyl)-N,3,4-trimethyl-2H-pyrazolo[3,4-d]pyridazin-7-amine,
2-(3-(3-amino-1-(2-fluorophenyl)propoxy)phenyl)-N,3,4-trimethyl-2H-pyrazolo[3,4-d]pyridazin-7-amine,
2-(3-(3-amino-1-phenylpropoxy)phenyl)-N,3,4-trimethyl-2H-pyrazolo[3,4-d]pyridazin-7-amine,
N,3,4-trimethyl-2-(3-(3-(methylamino)-1-phenylpropoxy)phenyl)-2H-pyrazolo[3,4-d]pyridazin-7-amine,
2-(3-(1-(2-fluorophenyl)-3-(methylamino)propoxy)phenyl)-3,4-dimethyl-2H-pyrazolo[3,4-d]pyridazin-7-amine,
3,4-dimethyl-2-(3-(3-(methylamino)-1-phenylpropoxy)phenyl)-2H-pyrazolo[3,4-d]pyridazin-7-amine,
2-(3-(3-amino-1-(2-fluorophenyl)propoxy)phenyl)-3,4-dimethyl-2H-pyrazolo[3,4-d]pyridazin-7-amine,
N,N,3,4-tetramethyl-2-(3-(piperidin-4-ylmethoxy)phenyl)-2H-pyrazolo[3,4-d]pyridazin-7-amine,
2-(3-(1-(2-Fluorophenyl)-3-(methylamino)propoxy)phenyl)-N,N,3,4-tetramethyl-2H-pyrazolo[3,4-d]pyridazin-7-amine,
2-((2-(3-(1-(2-fluorophenyl)-3-(methylamino)propoxy)phenyl)-3,4-dimethyl-2H-pyrazolo[3,4-d]pyridazin-7-yl)(methyl)amino)ethanol,
3-((2-(3-(1-(2-fluorophenyl)-3-(methylamino)propoxy)phenyl)-3,4-dimethyl-2H-pyrazolo[3,4-d]pyridazin-7-yl)(methyl)amino)propan-1-ol,
3-(2-fluorophenyl)-3-(3-(7-(3-methoxyazetidin-1-yl)-3,4-dimethyl-2H-pyrazolo[3,4-d]pyridazin-2-yl)phenoxy)-N-methylpropan-1-amine,
N,N,3,4-Tetramethyl-2-(3-(3-(methylamino)-1-(thiazol-2-yl)propoxy)phenyl)-2H-pyrazolo[3,4-d]pyridazin-7-amine,
N,N,3,4-tetramethyl-2-(3-(2-(methylamino)-1-phenylethoxy)phenyl)-2H-pyrazolo[3,4-d]pyridazin-7-amine,
2-(3-(1-(2-methoxyphenyl)-3-(methylamino)propoxy)phenyl)-N,N,3,4-tetramethyl-2H-pyrazolo[3,4-d]pyridazin-7-amine (*),
2-(3-(2-(dimethylamino)-1-phenylethoxy)phenyl)-N,N,3,4-tetramethyl-2H-pyrazolo[3,4-d]pyridazin-7-amine,
N,N,3,4-tetramethyl-2-(3-((4-(methylamino)-1-phenylbutan-2-yl)oxy)phenyl)-2H-pyrazolo[3,4-d]pyridazin-7-amine,
2-(3-(1-(3-methoxyphenyl)-3-(methylamino)propoxy)phenyl)-N,N,3,4-tetramethyl-2H-pyrazolo[3,4-d]pyridazin-7-amine (*),
N,N,3,4-tetramethyl-2-(3-(2-(methylamino)-1-(thiophen-2-yl)ethoxy)phenyl)-2H-pyrazolo[3,4-d]pyridazin-7-amine (*),
2-(3-(1-(2-fluorophenyl)-2-(methylamino)ethoxy)phenyl)-N,N,3,4-tetramethyl-2H-pyrazolo[3,4-d]pyridazin-7-amine,
N,N,3,4-tetramethyl-2-(3-(3-(methylamino)-1-(pyridin-3-yl)propoxy)phenyl)-2H-pyrazolo[3,4-d]pyridazin-7-amine,
2-(3-(1-(3-fluorophenyl)-2-(methylamino)ethoxy)phenyl)-N,N,3,4-tetramethyl-2H-pyrazolo[3,4-d]pyridazin-7-amine,
2-(3-((1-(benzyl(methyl)amino)-3-phenylpropan-2-yl)oxy)phenyl)-N,N,3,4-tetramethyl-2H-pyrazolo[3,4-d]pyridazin-7-amine,
N,N,3,4-tetramethyl-2-(3-(2-(methylamino)-1-(pyridin-3-yl)ethoxy)phenyl)-2H-pyrazolo[3,4-d]pyridazin-7-amine,
N,N,3,4-tetramethyl-2-(3-(3-(methylamino)-1-(4-(trifluoromethoxy)phenyl)propoxy)phenyl)-2H-pyrazolo[3,4-d]pyridazin-7-amine,
N,N,3,4-tetramethyl-2-(3-(2-(methylamino)-3-phenylpropoxy)phenyl)-2H-pyrazolo[3,4-d]pyridazin-7-amine,
N,N,3,4-tetramethyl-2-(3-(3-(methylamino)-1-(thiazol-5-yl)propoxy)phenyl)-2H-pyrazolo[3,4-d]pyridazin-7-amine,
N,N,3,4-tetramethyl-2-(3-(2-(1-(methylamino)cyclopropyl)-1-phenylethoxy)phenyl)-2H-pyrazolo[3,4-d]pyridazin-7-amine,
2-(3-(3-amino-1-(pyridin-3-yl)propoxy)phenyl)-N,N,3,4-tetramethyl-2H-pyrazolo[3,4-d]pyridazin-7-amine,
N,N,3,4-Tetramethyl-2-(3-(3-(methylamino)-1-(thiophen-2-yl)propoxy)phenyl)-2H-pyrazolo[3,4-d]pyridazin-7-amine,
2-(3-(2-(Dimethylamino)-1-(pyridin-3-yl)ethoxy)phenyl)-N,N,3,4-tetramethyl-2H-pyrazolo[3,4-d]pyridazin-7-amine,
N,N,3,4-Tetramethyl-2-(3-((1-phenethylpiperidin-4-yl)methoxy)phenyl)-2H-pyrazolo[3,4-d]pyridazin-7-amine,
2-(3-(3-((2-fluoroethyl)amino)-1-phenylpropoxy)phenyl)-N,N,3,4-tetramethyl-2H-pyrazolo[3,4-d]pyridazin-7-amine,
2-(3-(3-((2,2-difluoroethyl)amino)-1-phenylpropoxy)phenyl)-N,N,3,4-tetramethyl-2H-pyrazolo[3,4-d]pyridazin-7-amine,
3-(1-(3-(7-(Dimethylamino)-3,4-dimethyl-2H-pyrazolo[3,4-d]pyridazin-2-yl)phenoxy)-3-(methylamino)propyl)benzamide,
4-(1-(3-(7-(dimethylamino)-3,4-dimethyl-2H-pyrazolo[3,4-d]pyridazin-2-yl)phenoxy)-3-(methylamino)propyl)benzamide,
(S)-N,N,3,4-Tetramethyl-2-(3-(3-(methylamino)-1-phenylpropoxy)phenyl)-2H-pyrazolo[3,4-d]pyridazin-7-amine,
(R)-N,N,3,4-tetramethyl-2-(3-(3-(methylamino)-1-phenylpropoxy)phenyl)-2H-pyrazolo[3,4-d]pyridazin-7-amine,
(R)-N,N,3,4-Tetramethyl-2-(3-(2-(methylamino)-1-phenylethoxy)phenyl)-2H-pyrazolo[3,4-d]pyridazin-7-amine,
(S)-N,N,3,4-tetramethyl-2-(3-(2-(methylamino)-1-phenylethoxy)phenyl)-2H-pyrazolo[3,4-d]pyridazin-7-amine,
(R)-2-(3-(1-(2-Fluorophenyl)-3-(methylamino)propoxy)phenyl)-N,N,3,4-tetramethyl-2H-pyrazolo[3,4-d]pyridazin-7-amine,
(S)-2-(3-(1-(2-fluorophenyl)-3-(methylamino)propoxy)phenyl)-N,N,3,4-tetramethyl-2H-pyrazolo[3,4-d]pyridazin-7-amine,
(S)-N,N,3,4-tetramethyl-2-(3-(3-(methylamino)-1-(pyridin-3-yl)propoxy)phenyl)-2H-pyrazolo[3,4-d]pyridazin-7-amine,
(R)-N,N,3,4-tetramethyl-2-(3-(3-(methylamino)-1-(pyridin-3-yl)propoxy)phenyl)-2H-pyrazolo[3,4-d]pyridazin-7-amine,
3-(2-fluorophenyl)-N-methyl-3-(3-(1,4,5,7-tetramethyl-6H-pyrrolo[3,4-d]pyridazin-6-yl)phenoxy)propan-1-amine,
3-(3-fluorophenyl)-N-methyl-3-(3-(1,4,5,7-tetramethyl-6H-pyrrolo[3,4-d]pyridazin-6-yl)phenoxy)propan-1-amine,
(S)-3-(3-Fluorophenyl)-N-methyl-3-(3-(1,4,5,7-tetramethyl-6H-pyrrolo[3,4-d]pyridazin-6-yl)phenoxy)propan-1-amine and
(R)-3-(3-fluorophenyl)-N-methyl-3-(3-(1,4,5,7-tetramethyl-6H-pyrrolo[3,4-d]pyridazin-6-yl)phenoxy)propan-1-amine.

25. A process for the preparation of the compound of formula (I) according to claim 15, wherein when the group [CH₂]ₚR₂ is attached to the core structure through a carbon atom, said process comprises treating a compound of formula III 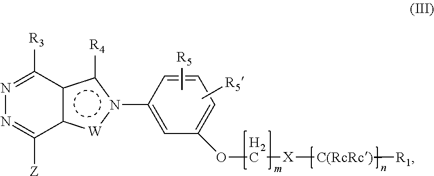
wherein Z represents a halogen, including chloro, or triflate with a suitable organometallic reagent of formula IVa, 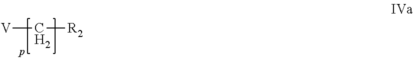
wherein V represents a suitable organometallic reagent, including a boron or zinc reagent,
or
when the group [CH₂]ₚR₂ is —NR₇R_{7′}, said process comprises treating a compound of formula III 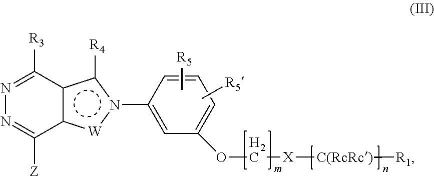
wherein Z represents a halogen, including chloro, or triflate with an amine of formula IVb
HNR₇R_{7′} IVb,
or
said process comprises treating a compound of formula VH 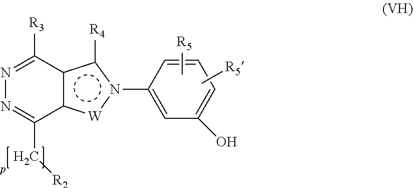
with a compound of formula VI 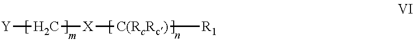
wherein Y represents a leaving group, including halogen, mesylate, tosylate, nosylate or triflate, or OH.

26. A process for the preparation of the compound according of formula (I) according to claim 15, employing a compound of Formula II, IIP, III, IIIP, IIIH, IVa, IVb, VP, VH, VI, VII, VIIP, VIII, IX, IXP, X, XI, XIP, IIa, IIaP, XII, XIII, XIVa, XIVb, XV, XVP, Ib, VbP or VbH 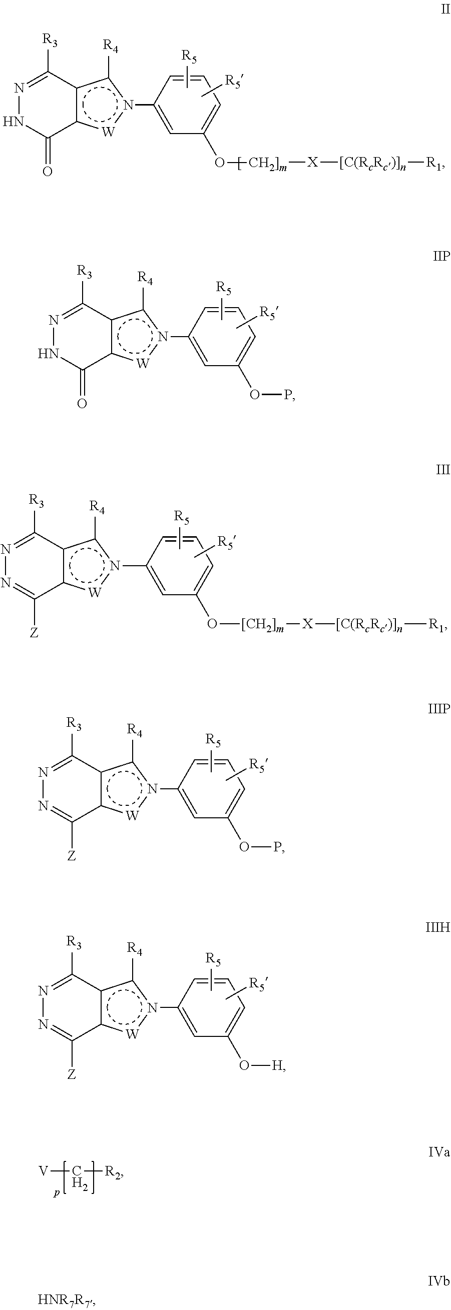 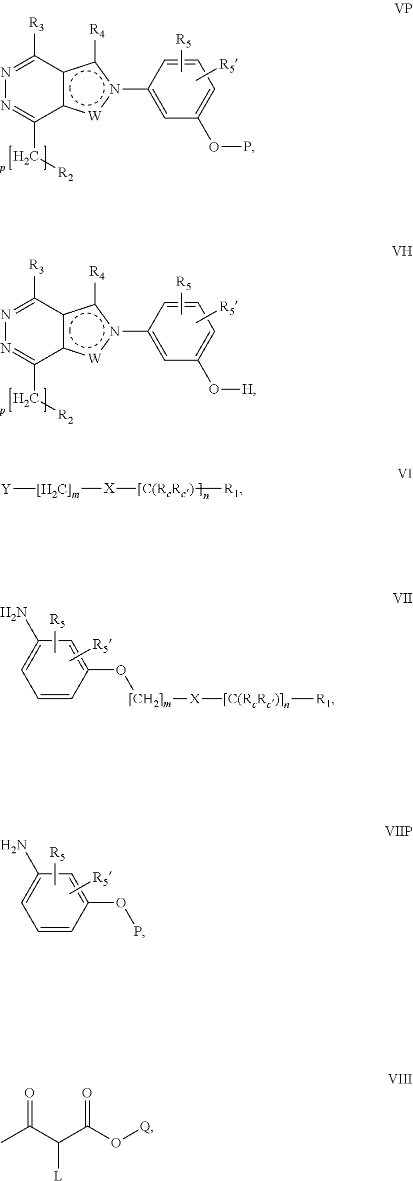 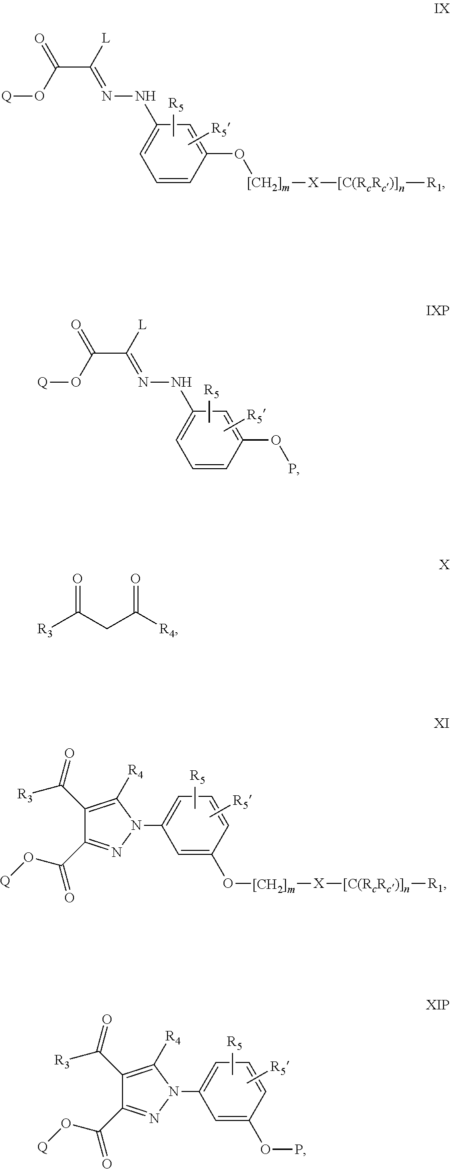 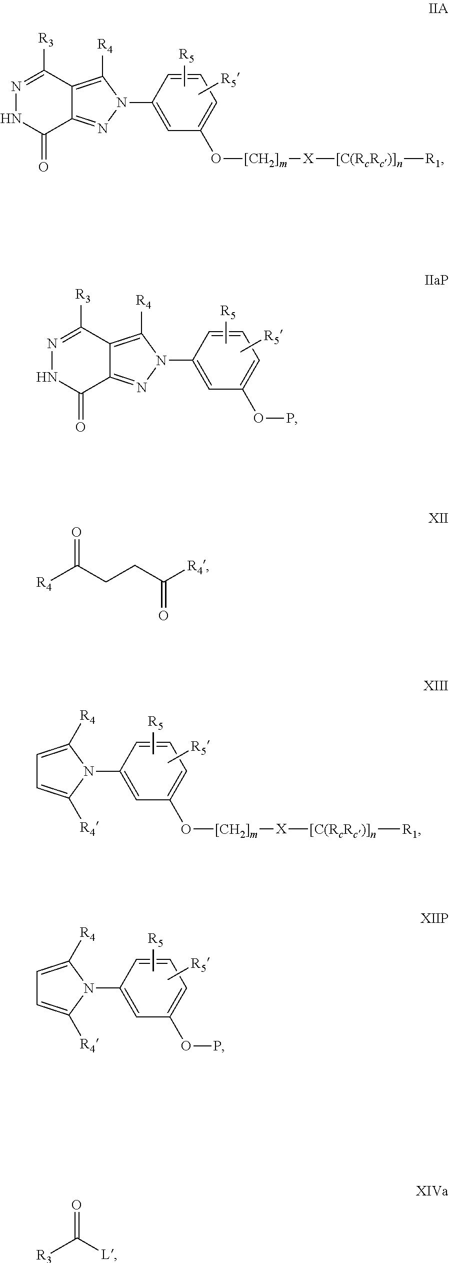 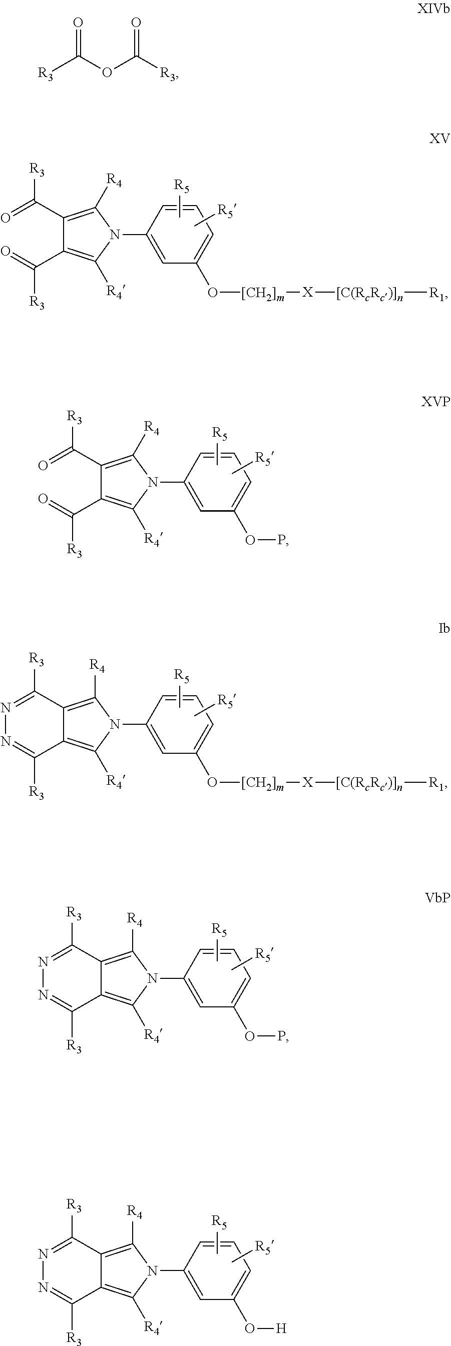
wherein R₁, R₂, R₃, R₄, R_{4′}, R₅, R_{5′}, R_{c}, R_{c′}, X, W, m, n and p are as defined in claim 15; Z represents a halogen, including chloro, or triflate; Y represents a leaving group, including halogen, mesylate, tosylate, nosylate or triflate, or OH; V represents an organometallic reagent, including a boron or zinc reagent; L and L′ represent halogen, including fluorine, chlorine, bromine or iodine; P represents a suitable protecting group, including alkyl, including methyl, or benzyl; and Q represents an alkyl group, including methyl or ethyl.

27. A pharmaceutical composition which comprises the compound according to claim 15, or a pharmaceutically acceptable salt thereof, and a pharmaceutically acceptable carrier, adjuvant or vehicle.

28. A method of treating pain in a subject in need thereof, comprising administration of an effective amount of the compound according to claim 15.

29. The method according to claim 28, wherein the pain is selected from the group consisting of medium to severe pain, visceral pain, chronic pain, cancer pain, migraine, inflammatory pain, acute pain or neuropathic pain, allodynia and hyperalgesia.
